(19) United States
(12) Patent Application Publication
(10) Pub. No.: US 2025/0101441 A1
(43) Pub. Date: Mar. 27, 2025

(54) MICROORGANISMS AND METHODS FOR PRODUCING CIS,CIS-MUCONIC ACID

(71) Applicant: Wisconsin Alumni Research Foundation, Madison, WI (US)

(72) Inventors: Timothy Donohue, Middleton, WI (US); Daniel Noguera, Madison, WI (US); Avery Vilbert, Madison, WI (US); Derek Gille, Chicago, IL (US); Wayne S. Kontur, Grove City, OH (US)

(73) Assignee: Wisconsin Alumni Research Foundation, Madison, WI (US)

(21) Appl. No.: 18/891,498

(22) Filed: Sep. 20, 2024

Related U.S. Application Data
(60) Provisional application No. 63/584,362, filed on Sep. 21, 2023.

(51) Int. Cl. C12N 15/74; C12N 9/02; C12N 9/10; C12N 9/88; C12N 9/90; C12P 7/44

(52) U.S. Cl. CPC C12N 15/74; C12N 9/0069; C12N 9/1085; C12N 9/88; C12N 9/90; C12P 7/44; C12Y 113/11001; C12Y 113/11002; C12Y 113/11008; C12Y 205/01; C12Y 401/01063; C12Y 503/03004; C12Y 505/01001

(57) ABSTRACT

Recombinant microorganisms configured for enhanced production of cis,cis-muconic acid and methods of using the recombinant microorganisms for the production of same.

### STATEMENT REGARDING FEDERALLY SPONSORED RESEARCH

This invention was made with government support under DE-SC0018409 awarded by the US Department of Energy. The government has certain rights in the invention.

### SEQUENCE LISTING

The instant application contains a Sequence Listing which has been submitted in XML format and is hereby incorporated by reference in its entirety. The XML copy, created Sep. 10, 2024, is named USPTO-09824551-P240032US02-SEQ_LIST and is 40,403 bytes in size.

### FIELD OF THE INVENTION

The invention is directed to recombinant microorganisms configured for enhanced production of cis, cis-muconic acid and methods of using the recombinant microorganisms for the production of same.

### BACKGROUND

One strategy to increase the environmental and economic sustainability of chemical production relies on harnessing the native or engineered metabolic pathways of microbes to catalyze the conversion of renewable resources into valuable products. Advances in genomics have enabled metabolic engineering approaches to convert abundant renewable resources into a number of targets for bioproduct production. One of these bio-privileged molecules is cis,cis-muconic acid (ccMA), which can be used as a precursor for the production of polymers including nylon-6,6, polyurethane, and polyethylene terephthalate (1). This dicarboxylic acid is an intermediate in the β-ketoadipic acid pathway of many bacteria and thus its production is amenable to metabolic engineering approaches.

The biological production of ccMA has been reported from food-grade, non-renewable sugars like glucose (2). Recently, significant attention has been drawn towards the production of ccMA from biomass lignin (3-6). Lignin is the most abundant renewable source of aromatics on the planet (7, 8) and accounts for approximately 20-30% (w/w) of dry biomass (9-11). However, lignin remains an underutilized industrial carbon source due to the chemical heterogeneity of the lignocellulose polymers. Lignin is composed of phenolic monomers that contain either 2 methoxy groups (S), 1 methoxy group (G) or no methoxy group (H) on the aromatic ring (12). Furthermore, biomass deconstruction methods used to recover aromatics produce a diverse set of aromatic monomers, dimers and oligomers (13).

Microbes capable of converting diverse biomass aromatics into simple products such as ccMA are needed.

### SUMMARY OF THE INVENTION

One aspect of the invention is directed to recombinant microorganisms comprising one or more modifications with respect to a corresponding microorganism not comprising the one or more modifications. In some versions, the one or more modifications comprise at least one of: a modification that increases flavin prenyltransferase activity with respect to the corresponding microorganism; a modification that increases protocatechuate decarboxylase activity with respect to the corresponding microorganism; a recombinant protocatechuate decarboxylase D gene encoding a protocatechuate decarboxylase D protein; a modification that increases catechol 1,2-dioxygenase activity with respect to the corresponding microorganism; a modification that decreases muconate lactonizing enzyme activity with respect to the corresponding microorganism; a modification that decreases muconolactone isomerase activity with respect to the corresponding microorganism; a modification that decreases catechol 2,3-dioxygenase activity with respect to the corresponding microorganism; and a modification that decreases protocatechuate 4,5-dioxygenase activity with respect to the corresponding microorganism.

In some versions, the one or more modifications comprise one or more genetic modifications in the recombinant microorganism with respect to the corresponding microorganism.

In some versions, the one or more modifications comprise the modification that increases flavin prenyltransferase activity with respect to the corresponding microorganism. In some versions, the modification that increases flavin prenyltransferase activity with respect to the corresponding microorganism, if present in the recombinant microorganism, comprises a recombinant gene encoding a flavin prenyltransferase. In some versions, the modification that increases flavin prenyltransferase activity with respect to the corresponding microorganism, if present in the recombinant microorganism, comprises a recombinant gene encoding a flavin prenyltransferase comprising a sequence having at least 80%, at least 85%, at least 90%, at least 95%, or at least 99% sequence identity to SEQ ID NO:2.

In some versions, the one or more modifications comprise the modification that increases protocatechuate decarboxylase activity with respect to the corresponding microorganism. In some versions, the modification that increases protocatechuate decarboxylase activity with respect to the corresponding microorganism, if present in the recombinant microorganism, comprises a recombinant gene encoding a protocatechuate decarboxylase. In some versions, the modification that increases protocatechuate decarboxylase activity with respect to the corresponding microorganism, if present in the recombinant microorganism, comprises a recombinant gene encoding a protocatechuate decarboxylase comprising a sequence having at least 80%, at least 85%, at least 90%, at least 95%, or at least 99% sequence identity to SEQ ID NO:4.

In some versions, the one or more modifications comprise the recombinant protocatechuate decarboxylase D gene. In some versions, the recombinant protocatechuate decarboxylase D gene, if present in the recombinant microorganism, encodes a protocatechuate decarboxylase D protein comprising a sequence having at least 80%, at least 85%, at least 90%, at least 95%, or at least 99% sequence identity to any one of SEQ ID NOS:6, 24, and 26. In some versions, the recombinant protocatechuate decarboxylase D gene, if present in the recombinant microorganism, encodes a protocatechuate decarboxylase D protein comprising a sequence having at least 80%, at least 85%, at least 90%, at least 95%, or at least 99% sequence identity to SEQ ID NO:6.

In some versions, the one or more modifications comprise the modification that increases catechol 1,2-dioxygenase activity with respect to the corresponding microorganism. In some versions, the modification that increases catechol 1,2-dioxygenase activity with respect to the corresponding microorganism, if present in the recombinant microorganism, comprises a recombinant gene encoding a catechol 1,2-dioxygenase. In some versions, the modification that increases catechol 1,2-dioxygenase activity with respect to the corresponding microorganism, if present in the recombinant microorganism, comprises a recombinant gene encoding a catechol 1,2-dioxygenase comprising a sequence having at least 80%, at least 85%, at least 90%, at least 95%, or at least 99% sequence identity to SEQ ID NO:8.

In some versions, the one or more modifications comprise the modification that decreases muconate lactonizing enzyme activity with respect to the corresponding microorganism. In some versions, the modification that decreases muconate lactonizing enzyme activity with respect to the corresponding microorganism, if present in the recombinant microorganism, comprises a mutation to a gene in the corresponding microorganism encoding a muconate lactonizing enzyme. In some versions, the modification that decreases muconate lactonizing enzyme activity with respect to the corresponding microorganism, if present in the recombinant microorganism, comprises a mutation to a gene in the corresponding microorganism encoding a muconate lactonizing enzyme comprising a sequence having at least 80%, at least 85%, at least 90%, at least 95%, or at least 99% sequence identity to SEQ ID NO:10.

In some versions, the one or more modifications comprise the modification that decreases muconolactone isomerase activity with respect to the corresponding microorganism. In some versions, the modification that decreases muconolactone isomerase activity with respect to the corresponding microorganism, if present in the recombinant microorganism, comprises a mutation to a gene in the corresponding microorganism encoding a muconolactone isomerase. In some versions, the modification that decreases muconolactone isomerase activity with respect to the corresponding microorganism, if present in the recombinant microorganism, comprises a mutation to a gene in the corresponding microorganism encoding a muconolactone isomerase comprising a sequence having at least 80%, at least 85%, at least 90%, at least 95%, or at least 99% sequence identity to SEQ ID NO:12.

In some versions, the one or more modifications comprise the modification that decreases catechol 2,3-dioxygenase activity with respect to the corresponding microorganism. In some versions, the modification that decreases catechol 2,3-dioxygenase activity with respect to the corresponding microorganism, if present in the recombinant microorganism, comprises a mutation to a gene in the corresponding microorganism encoding a catechol 2,3-dioxygenase. In some versions, the modification that decreases catechol 2,3-dioxygenase activity with respect to the corresponding microorganism, if present in the recombinant microorganism, comprises a mutation to a gene in the corresponding microorganism encoding a catechol 2,3-dioxygenase comprising a sequence having at least 80%, at least 85%, at least 90%, at least 95%, or at least 99% sequence identity to SEQ ID NO:14.

In some versions, the one or more modifications comprise the modification that decreases protocatechuate 4,5-dioxygenase activity with respect to the corresponding microorganism. In some versions, the modification that decreases protocatechuate 4,5-dioxygenase activity with respect to the corresponding microorganism, if present in the recombinant microorganism, comprises a mutation to a gene in the corresponding microorganism encoding a protocatechuate 4,5-dioxygenase subunit. In some versions, the modification that decreases protocatechuate 4,5-dioxygenase activity with respect to the corresponding microorganism, if present in the recombinant microorganism, comprises a mutation to any one, two, three, or each of: a gene in the corresponding microorganism encoding a protocatechuate 4,5-dioxygenase subunit comprising a sequence having at least 80%, at least 85%, at least 90%, at least 95%, or at least 99% sequence identity to SEQ ID NO:16; a gene in the corresponding microorganism encoding a protocatechuate 4,5-dioxygenase subunit comprising a sequence having at least 80%, at least 85%, at least 90%, at least 95%, or at least 99% sequence identity to SEQ ID NO:18; a gene in the corresponding microorganism encoding a protocatechuate 4,5-dioxygenase subunit comprising a sequence having at least 80%, at least 85%, at least 90%, at least 95%, or at least 99% sequence identity to SEQ ID NO:20; and a gene in the corresponding microorganism encoding a protocatechuate 4,5-dioxygenase subunit comprising a sequence having at least 80%, at least 85%, at least 90%, at least 95%, or at least 99% sequence identity to SEQ ID NO: 22.

In some versions, the recombinant microorganism exhibits enhanced production of cis, cis-muconic acid with respect to the corresponding microorganism.

In some versions, the recombinant microorganism is a bacterium. In some versions, the recombinant microorganism is a phenol-degrading microorganism. In some versions, the recombinant microorganism is from the genus Novosphingobium. In some versions, the recombinant microorganism is Novosphingobium aromaticivorans.

Another aspect of the invention is directed to methods for producing cis,cis-muconic acid. In some versions, the methods comprise culturing the recombinant microorganism of the invention in a medium. In some versions, the medium comprises a plant-derived phenolic. In some versions, the medium comprises a plant-derived phenolic selected from the group consisting of a syringyl phenolic, a guaiacyl phenolic, and a p-hydroxyphenyl phenolic. In some versions, the medium comprises depolymerized lignin. Some versions further comprise isolating the cis,cis-muconic acid from the medium and/or the recombinant microorganism.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Metabolic pathways for aromatic catabolism from H- and G-phenolic monomers (left) and other aromatics (benzoic acid, phenol and guaiacol, right). Two central intermediates, protocatechuic acid (PCA) and catechol, are shown.

FIG. 2. Chromatogram of the indicated aromatics used in LC Method 1 (see Examples) to quantify the aromatics at 263 nm.

FIG. 3. Chromatogram of the commercial compounds used as aromatics to quantify the aromatics present in Qsub poplar APL measured at 263 nm with chromatographic Method 2. The aromatics measured with this method (1 mM final concentration) are shown and labeled above each peak with protocatechuic acid (PCA), cis,cis-muconic acid (ccMA), 4-hydroxybenzoic acid (4-HBA), vanillic acid, vanillin and 4-coumaric acid (4-CA) and ferulic acid.

FIGS. 4A-4C. Cell density (FIG. 4A) and extracellular concentrations of vanillic acid (FIG. 4B) and protocatechuic acid (PCA) (FIG. 4C) in cultures of the parent strain 12444, the 12444_ΔligAB1/2 strain, and the ΔligAB1/2 ΔnadBCD strain (12444_PCA). Cells were grown in batch cultures in minimal media containing 2 mM vanillic acid and 10 mM glucose. No other aromatics were detected in the media. All experiments were performed in triplicate. Error bars represent 1 standard deviation above and below the mean.

FIGS. 5A-5C. Representative HPLC traces of extracellular aromatics using LC Method 1 from N. aromaticivorans strains 12444 (FIG. 5A), ΔligAB1/2 (FIG. 5B) or 12444_PCA (FIG. 5C) grown with 2 mM vanillic acid and 10 mM glucose as carbon sources. The traces representing the sample taken at T=0 hr, T=14 hr, T=25 hr, and T=50 hr are labeled. All identified aromatics are labeled.

FIGS. 6A and 6B. SDS-PAGE gel of purified recombinant NadCD (FIG. 6A), EcAroY/D (FIG. 6B). The box highlights the purified protein.

FIG. 7. The dependence of NadCD on a prFMN source for PCA decarboxylase activity. Experiments were performed with 100 nM NadCD with 1 mM PCA in either the presence or absence of a prFMN lysate. The bars represent the concentration of either PCA (P) or catechol (C) after 18 hr of incubation at room temperature. A control of lysate with 1 mM PCA was also performed to ensure no PCA to catechol conversion with lysate only. Both enzymes only produced catechol (C) from PCA (P) when in the presence of a prFMN source. Each bar is the average of 3 trials with error bars representing one standard deviation.

FIGS. 8A and 8B. Time-dependent conversion of PCA to catechol by either 100 nM NadCD (FIG. 8A) or 100 nM EcAroY (FIG. 8B) with 1 mM PCA in prFMN lysate in 50 mM HEPES, 150 mM NaCl pH 7.5. All experiments were performed in triplicate with each data point representing the average of 3 trials with error bars representing one standard deviation.

FIGS. 9A-9C. Cell density (FIG. 9A) and extracellular metabolite concentration of vanillic acid (FIG. 9B) and protocatechuic acid (PCA) (FIG. 9C) of LigAB1_NaDec and LigAB1_EcDec. Cultures were grown in triplicate with 2 mM vanillic acid and 10 mM glucose, and metabolite concentrations were analyzed by LCMS. The data points are the average of 3 trials with error bars representing one standard deviation.

FIGS. 10A and 10B. Representative HPLC traces of extracellular aromatics using LC Method 1 from N. aromaticivorans strains LigAB1_NaDec (FIG. 10A) or LigAB1_EcDec (FIG. 10B) grown with 2 mM vanillic acid and 10 mM glucose as carbon sources. The traces representing the sample taken at T=0 hr, T=9 hr, T=12 hr, T=24 hr, and T=48 hr are labeled. All identified aromatics are labeled.

FIGS. 11A and 11B. SDS-PAGE gel of purified recombinant NaCatA (FIG. 11A) and EcCatA (FIG. 11B). The box highlights the purified protein.

FIG. 12. Catechol to ccMA production by NaCatA. The reaction was initiated with 0.5 μM enzyme with either 25 μM or 50 μM catechol in 50 mM HEPES 150 mM NaCl pH 7.5. The production of ccMA was measured by the increase in absorbance at 260 nm.

FIGS. 13A and 13B. Representative time courses of the formation of ccMA with either NaCatA (FIG. 13A) or EcCatA (FIG. 13B). Both reactions were performed in 50 mM HEPES 150 mM NaCl pH 7.5 with either 0.5 μM NaCatA (FIG. 13A) or EcCatA (FIG. 13B) and initiated with 100 μM catechol. The formation of ccMA was monitored by UV/vis absorption spectroscopy at A260-nm and the data points were best fit to a linear equation ([ccMA]=kt+[ccMA]0). The resulting fit is shown as the black line. The inset shows the average rate after 3 trials with error represented by 1 standard deviation above and below the mean.

FIG. 14. Relative transcript abundance of catA (Saro_3830) and xylE (Saro_3857) when N. aromaticivorans strain 12444 is grown with either glucose (G), protocatechuic acid (P) or vanillic acid (V) as a carbon source.

FIGS. 15A-15D. Cell density (FIG. 15A) of NaDec_ccMA and EcDec_ccMA and extracellular metabolite concentration of protocatechuic acid (PCA) (FIG. 15B), catechol (FIG. 15C) and ccMA (FIG. 15D). Cultures were grown in triplicate in a shake-flasks with 2 mM PCA and 10 mM glucose. Metabolite concentrations were analyzed by LCMS. Each data point represents the average of 3 trials with error bars representing one standard deviation.

FIGS. 16A and 16B. Representative HPLC traces of extracellular aromatics using LC Method 2 (see Examples) from N. aromaticivorans strains of NaDec_ccMA (FIG. 16A) or EcDec_ccMA (FIG. 16B) grown with 2 mM protocatechuic acid (PCA) and 10 mM glucose. The black trace represents the sample taken at T=0 and the blue trace represents the sample taken at T=72 hr. All identified aromatics are labeled.

FIGS. 17A and 17B. Aromatics identified in Qsub Poplar APL before (B) and after (A) acid hydrolysis. The HPLC trace (FIG. 17A) is shown with the identified aromatics protocatechuic acid (PCA), vanillic acid, and 4-hydroxybenzoic acid (4-HBA). The corresponding calculated concentrations of aromatics identified before (left bar shown for each compound) and after (right bar shown for each compound) acid hydrolysis are shown in FIG. 17B.

FIG. 18. Conversion of Qsub APL into ccMA with either EcDec_ccMA or NaDec_ccMA N. aromaticivorans strains. The first bar represents the total concentration of the major free and glycosylated aromatics identified from Qsub APL with PCA (top portion), vanillic acid (middle portion) and 4-hydroxybenzoic acid (bottom portion). The second and third bars represent the concentration of ccMA produced from either the EcDec_ccMA or NaDec_ccMA strains after 48 h incubation with Qsub APL. Each bar is the average of 3 trials with error bars representing one standard deviation.

FIGS. 19A and 19B. Representative HPLC traces of extracellular aromatics using LC Method 2 from N. aromaticivorans strains NaDec_ccMA (FIG. 19A) or EcDec_ccMA (FIG. 19B) grown with Qsub APL popular as a carbon source. The respective traces representing the sample taken at T=0 hr and T=48 hr are labeled. All identified aromatics are labeled.

### DETAILED DESCRIPTION OF THE INVENTION

One aspect of the invention is directed to recombinant microorganisms. The recombinant microorganisms of the invention can be configured for enhanced production of cis,cis-muconic acid or other compounds. The recombinant microorganisms of the invention comprise one or more modifications that increase the activity of one or more genes or gene products, decrease the activity of one or more genes or gene products, or increase the activity of one or more genes or gene products and decrease the activity of one or more genes or gene products. The recombinant microorganisms with the modifications can exhibit enhanced production of cis,cis-muconic acid with respect to corresponding microorganisms not comprising the modifications.

“Modifications that increase the activity of one or more genes or gene products” refers to any modification to microorganism that increases expression of a gene in producing its gene product or increases the functioning of the gene product. “Increase” in this context encompasses increasing beyond a baseline activity. The baseline activity can be a positive baseline activity or null activity. Exemplary modifications that increase the activity of one or more genes or gene products include genetic modifications. The genetic modifications include genetic modifications to a gene in a manner that increases expression of the gene in producing the gene product. Such modifications include operationally connecting the coding sequence to a stronger promoter or enhancer, etc., and/or introducing additional copies of the gene (whether the native gene or a recombinant version). The genetic modifications also include mutations to a first gene (such as a transcription factor or an inhibitor of a transcription factor) that affects the expression of a second gene. The genetic modifications also include one or more copies of an exogenous or heterologous gene introduced into the microorganism. Other genetic modifications are described herein.

“Modifications that decrease the activity of one or more genes or gene products” refers to any modification to a microorganism that decreases expression of the gene and thus production of the gene product and/or decreases the functioning of the gene product per se. “Decrease” in this context encompasses reducing below a positive baseline level of expression and/or activity to a lower level of expression and/or activity. The lower level of expression and/or activity can be a lower positive level of expression and/or activity or null expression and/or activity. Decreasing the functioning of a gene product may comprise decreasing the specific activity of a gene product. Exemplary modifications that decrease the activity of one or more genes or gene products include genetic modifications. Exemplary genetic modifications include mutations to a gene that decrease expression of the gene in producing the gene product. Such mutations may include mutations to the coding sequence, the promoter, an enhancer, any other part of the gene, or the entire gene. Other exemplary genetic modifications include mutations to the coding sequence of a gene that decrease the functioning of a gene product expressed from the gene. Exemplary mutations include substitutions, insertions, and deletions, including partial and full deletions of a particular gene. Other exemplary genetic modifications include recombinant nucleotide sequences configured to express antisense RNAs or other molecules that decrease production of a gene product. Other exemplary genetic modifications include mutations to a first gene (such as a transcription factor or an inhibitor of a transcription factor) that affects the expression of a second gene. Other exemplary genetic modifications are described elsewhere herein. Other modifications include epigenetic modifications, such as methylation, etc.

“Corresponding microorganism” refers to a microorganism of the same species having the same or substantially same genetic and proteomic composition as a recombinant microorganism of the invention, with the exception of genetic and proteomic differences resulting from the modifications specified herein for the recombinant microorganisms of the invention in a given particular embodiment. In some versions, the corresponding microorganism is the native version of the recombinant microorganism of the invention, i.e., the unmodified microorganism as found in nature. The terms “microorganism” and “microbe” are used interchangeably herein.

In some versions, the recombinant microorganisms comprise one or more modifications with respect to a corresponding microorganism not comprising the one or more modifications. The one or more modifications can comprise, in any combination, a modification that increases flavin prenyltransferase activity with respect to the corresponding microorganism, a modification that increases protocatechuate decarboxylase activity with respect to the corresponding microorganism, a recombinant protocatechuate decarboxylase D gene encoding a protocatechuate decarboxylase D protein, a modification that increases catechol 1,2-dioxygenase activity with respect to the corresponding microorganism, a modification that decreases muconate lactonizing enzyme activity with respect to the corresponding microorganism, a modification that decreases muconolactone isomerase activity with respect to the corresponding microorganism, a modification that decreases catechol 2,3-dioxygenase activity with respect to the corresponding microorganism, and/or a modification that decreases protocatechuate 4,5-dioxygenase activity with respect to the corresponding microorganism.

Flavin prenyltransferase activity is characterized by EC 2.5.1.129 and comprises the ability to catalyze the addition of dimethylallyl-monophosphate (DMAP) (or dimethylallyl-pyrophosphate (DMAPP)) onto the N5 and C6 positions of flavin mononucleotide (FMN) to result in the formation of the prenylated FMN (prFMN) cofactor. Flavin prenyltransferase activity is performed by flavin prenyltransferases. An exemplary flavin prenyltransferase is nadB/NadB (Saro_3873) of Novosphingobium aromaticivorans, the nucleic acid coding sequence of which is SEQ ID NO:1 and the protein sequence of which is SEQ ID NO:2. Other exemplary flavin prenyltransferases include proteins with flavin prenyltransferase activity having a sequence at least 80%, at least 85%, at least 90%, at least 95%, or at least 99% identical to SEQ ID NO:2. Other exemplary flavin prenyltransferases include the EcdB enzyme from Enterobacter cloacae and the KpdB enzyme from Klebsiella pneumoniae described in the following examples, as well as modified versions thereof. Other flavin prenyltransferases are known in the art. In some versions of the invention, the one or more modifications in the recombinant microorganisms can comprise a genetic modification that increases flavin prenyltransferase activity. A genetic modification that increases flavin prenyltransferase activity can comprise a recombinant flavin prenyltransferase gene. In some versions, the recombinant flavin prenyltransferase gene is an exogenous recombinant flavin prenyltransferase gene newly introduced to the microorganism. In some versions, the recombinant flavin prenyltransferase gene is a modified form of an endogenous flavin prenyltransferase gene already present in the microorganism.

Protocatechuate decarboxylase activity is characterized by EC 4.1.1.63 and comprises the ability to catalyze conversion of catalyzes the chemical reaction 3,4-dihydroxybenzoate to catechol CO₂. Protocatechuate decarboxylase activity is performed by protocatechuate decarboxylases. An exemplary protocatechuate decarboxylase is nadC/NadC (Saro_3877) of Novosphingobium aromaticivorans, the nucleic acid coding sequence of which is SEQ ID NO:3 and the protein sequence of which is SEQ ID NO:4. Other exemplary protocatechuate decarboxylases include proteins with protocatechuate decarboxylase activity having a sequence at least 80%, at least 85%, at least 90%, at least 95%, or at least 99% identical to SEQ ID NO:4. Other exemplary protocatechuate decarboxylases include the EcdC and EcAroY enzymes from Enterobacter cloacae and the KpdC and KpAroY enzymes from Klebsiella pneumoniae described in the following examples, as well as modified versions thereof. Other protocatechuate decarboxylases are known in the art. In some versions of the invention, the one or more modifications in the recombinant microorganisms can comprise a genetic modification that increases protocatechuate decarboxylase activity. A genetic modification that increases protocatechuate decarboxylase activity can comprise a recombinant protocatechuate decarboxylase gene. In some versions, the recombinant protocatechuate decarboxylase gene is an exogenous protocatechuate decarboxylase gene newly introduced to the microorganism. In some versions, the recombinant protocatechuate decarboxylase gene is a modified form of an endogenous protocatechuate decarboxylase gene already present in the microorganism.

Protocatechuate decarboxylase D genes are genes encoding protocatechuate decarboxylase D proteins. Protocatechuate decarboxylase D genes are often found in aromatic-degrading microbes (e.g., Novosphingobium aromaticivorans, Klebsiella pneumoniae, Enterobacter cloacae, Pseudomonas putida, Sedimentibacter hydroxybenzoicus, Streptomyces sp. D7, Bacillus subtilis, B. licheniformis, E. coli O157:H7, Shigella dysenteriae, Salmonella enterica, S. paratyphi, S. typhimurium, S. bongori, and S. diarizonae, among others), are clustered in a single operon with flavin prenyltransferase and protocatechuate decarboxylase genes, have an open reading from overlapping with—or just downstream of—the protocatechuate decarboxylase gene, and encode a protein of about 70-80 amino acids (38). Such clusters are known in the art as hydroxyarylic acid decarboxylases (38). Exemplary protocatechuate decarboxylase D genes and proteins include nadD/NadD (Saro_3878) of Novosphingobium aromaticivorans (SEQ ID NOS:5 and 6), ecdD/EcdD of Enterobacter cloacae (SEQ ID NOS:23 and 24), and kpdD/KpdD of Klebsiella pneumoniae (SEQ ID NOS:25 and 26). Other protocatechuate decarboxylases are known in the art (Johnson C W, Salvachda D, Khanna P, Smith H, Peterson D J, Beckham G T. Enhancing muconic acid production from glucose and lignin-derived aromatic compounds via increased protocatechuate decarboxylase activity. Metab Eng Commun. 2016 Apr. 22; 3:111-119) (Payer S E, Marshall S A, Barland N, Sheng X, Reiter T, Dordic A, Steinkellner G, Wuensch C, Kaltwasser S, Fisher K, Rigby S E J, Macheroux P, Vonck J, Gruber K, Faber K, Himo F, Leys D, Pavkov-Keller T, Glueck S M. Regioselective para-Carboxylation of Catechols with a Prenylated Flavin Dependent Decarboxylase. Angew Chem Int Ed Engl. 2017 Oct. 23; 56(44):13893-13897) (Sonoki T, Morooka M, Sakamoto K, Otsuka Y, Nakamura M, Jellison J, Goodell B. Enhancement of protocatechuate decarboxylase activity for the effective production of muconate from lignin-related aromatic compounds. J Biotechnol. 2014 Dec. 20; 192 Pt A:71-7) (Lupa B, Lyon D, Gibbs M D, Reeves R A, Wiegel J. Distribution of genes encoding the microbial non-oxidative reversible hydroxyarylic acid decarboxylases/phenol carboxylases. Genomics. 2005 September; 86(3):342-51). In some versions of the invention, the one or more modifications in the recombinant microorganisms comprise a recombinant protocatechuate decarboxylase D gene. In some versions, the recombinant protocatechuate decarboxylase D gene encode a protocatechuate decarboxylase D protein having at least 80%, at least 85%, at least 90%, at least 95%, at least 99%, or 100% sequence identity to any one of SEQ ID NOS:6, 24, and 26. In some versions, the recombinant protocatechuate decarboxylase D gene is an exogenous protocatechuate decarboxylase D gene newly introduced to the microorganism. In some versions, the recombinant protocatechuate decarboxylase D gene is a modified form of an endogenous protocatechuate decarboxylase gene already present in the microorganism.

Catechol 1,2-dioxygenase activity is characterized by EC 1.13.11.1 and comprises the ability to catalyze the oxidative ring cleavage of catechol to form cis,cis-muconic acid. Catechol 1,2-dioxygenase activity is performed by catechol 1,2-dioxygenases. An exemplary catechol 1,2-dioxygenase is catA/CatA (Saro_3830) of Novosphingobium aromaticivorans, the nucleic acid coding sequence of which is SEQ ID NO:7 and the protein sequence of which is SEQ ID NO:8. Other exemplary catechol 1,2-dioxygenases include proteins with catechol 1,2-dioxygenase activity having a sequence at least 80%, at least 85%, at least 90%, at least 95%, or at least 99% identical to SEQ ID NO:8. Other exemplary catechol 1,2-dioxygenases include the CatA enzymes from Enterobacter cloacae and Pseudomonas putida described in the following examples, as well as modified versions thereof. Other catechol 1,2-dioxygenases are known in the art, including those found in Pseudomonas sp. (Dorn E, Knackmuss H J. Chemical structure and biodegradability of halogenated aromatic compounds. Two catechol 1,2-dioxygenases from a 3-chlorobenzoate-grown pseudomonad. Biochem J. 1978 Jul. 15; 174(1):73-84), Pseudomonasfluorescens (Hayaishi O, Katagiri M, Rothberg S. Studies on oxygenases; pyrocatechase. J Biol Chem. 1957 December; 229(2):905-20.), Aspergillus niger (Ninnekar H, Vaidyanathan C. Catechol 1,2-dioxygenase from Aspergillus niger: Purification and properties. J. Indian Inst. Sci. 1981 63C:131-136), Brevibacterium fuscum (Nakagawa H, Inoue H, Takeda Y. Characteristics of Catechol Oxygenase from Brevibacterium fuscum. J Biochem. 1963 July; 54:65-74) (Hou C T, Patel R, Lillard M O. Extradiol cleavage of 3-methylcatechol by catechol 1,2-dioxygenase from various microorganisms. Appl Environ Microbiol. 1977 March; 33(3):725-7), Acinetobacter calcoaceticus (Patel R N, Hou C T, Felix A, Lillard M O. Catechol 1,2-dioxygenase from Acinetobacter calcoaceticus: purification and properties. J Bacteriol. 1976 July; 127(1):536-44), Trichosporon cutaneum (Itoh, M. Characteristics of a new catechol-1,2-oxygenase from Trichosporon cutaneum WY2-2. Agric. Biol. Chem. 1981 45(1):2787-2796), Rhodococcus erythropolis (Murakami S, Kodama N, Shinke R, Aoki K. Classification of catechol 1,2-dioxygenase family: sequence analysis of a gene for the catechol 1,2-dioxygenase showing high specificity for methylcatechols from Gram+ aniline-assimilating Rhodococcus erythropolis AN-13. Gene. 1997 Jan. 31; 185(1):49-54), Frateuria sp. (Aoki K, Konohana T, Shinke R, Nishira H. Two catechol 1,2-dioxygenases from aniline-assimilating bacterium, Frateuria species ANA-18. Agric. Biol. Chem. 1984 48(1):2097-2104), Rhizobium trifolii (Chen Y, Glenn A, Dilworth M. Aromatic metabolism in Rhizobium trifolii-catechol 1,2-dioxygenase. Arch. Microbiol. 1985 141(1):225-228), Pseudomonas putida (Pascal RA Jr, Huang DS. Reactions of 3-ethylcatechol and 3-(methylthio)catechol with catechol dioxygenases. Arch Biochem Biophys. 1986 July; 248(1):130-7), Candida tropicalis (Krug M, Straube G. Degradation of phenolic compounds by the yeast Candida tropicalis HP 15. II. Some properties of the first two enzymes of the degradation pathway. J Basic Microbiol. 1986; 26(5):271-81), Candida maltose (Gomi K, Horiguchi. Purification and characterization of pyrocatechase from the catechol-assimilating yeast Candida maltose. Agric. Biol. Chem. 1988 52(2):585-587), Rhizobium leguminosarum (Chen Y P, Lovell C R. Purification and Properties of Catechol 1,2-Dioxygenase from Rhizobium leguminosarum biovar viceae USDA 2370. Appl Environ Microbiol. 1990 June; 56(6):1971-3), and Nocardia sp. (Smith M, Ratledge C, Crook S. Properties of cyanogen bromide-activated, Agarose-immobilized catechol 1,2-dioxygenase from freeze-dried extracts of Nocardia sp. NCIB 10503. Enzyme Microb. Technol. 1990 12(12):945-949). In some versions of the invention, the one or more modifications in the recombinant microorganisms can comprise a genetic modification that increases catechol 1,2-dioxygenase activity. A genetic modification that increases catechol 1,2-dioxygenase activity can comprise a recombinant catechol 1,2-dioxygenase gene. In some versions, the recombinant catechol 1,2-dioxygenase gene is an exogenous catechol 1,2-dioxygenase gene newly introduced to the microorganism. In some versions, the recombinant catechol 1,2-dioxygenase gene is a modified form of an endogenous catechol 1,2-dioxygenase gene already present in the microorganism.

Muconate lactonizing enzyme activity is characterized by EC 5.5.1.1 and comprises the ability to catalyze the conversion of cis,cis-muconate to (+)-muconolactone ((S)-5-oxo-2,5-dihydro-2-furylacetate, (S)-muconolactone, 2-[(2S)-5-oxo-2H-furan-2-yl]acetate, [(2S)-5-oxo-2,5-dihydrofuran-2-yl]acetate). Muconate lactonizing enzyme activity is performed by muconate lactonizing enzymes. An exemplary muconate lactonizing enzyme is catB/CatB (Saro_3828) of Novosphingobium aromaticivorans, the nucleic acid coding sequence of which is SEQ ID NO:9 and the protein sequence of which is SEQ ID NO: 10. Other exemplary muconate lactonizing enzymes include proteins with muconate lactonizing enzyme activity having a sequence at least 80%, at least 85%, at least 90%, at least 95%, or at least 99% identical to SEQ ID NO:10. Other exemplary muconate lactonizing enzymes include the CatB enzymes from Enterobacter cloacae and Pseudomonas putida described in the following examples, as well as modified versions thereof. Other muconate lactonizing enzymes are known in the art. In some versions of the invention, the one or more modifications in the recombinant microorganisms can comprise a genetic modification that decreases muconate lactonizing enzyme activity. A genetic modification that decreases muconate lactonizing enzyme activity can comprise a genetic modification to a muconate lactonizing enzyme gene. A genetic modification to a muconate lactonizing enzyme gene can comprise a substitution or insertion in or a complete or partial deletion of the muconate lactonizing enzyme gene.

Muconolactone isomerase (muconolactone A-isomerase) activity is characterized by EC 5.3.3.4 and comprises the ability to catalyze the conversion of (+)-muconolactone to beta-ketoadipate-enol-lactone (5-oxo-4,5-dihydro-2-furylacetate, (5-oxo-4,5-dihydrofuran-2-yl)acetate, (4,5-dihydro-5-oxofuran-2-yl)-acetate, enol-lactone). Muconolactone isomerase activity is performed by muconate lactonizing enzymes. An exemplary muconolactone isomerase is catC/CatC (Saro_3829) of Novosphingobium aromaticivorans, the nucleic acid coding sequence of which is SEQ ID NO:11 and the protein sequence of which is SEQ ID NO:12. Other exemplary muconolactone isomerases include proteins with muconolactone isomerase activity having a sequence at least 80%, at least 85%, at least 90%, at least 95%, or at least 99% identical to SEQ ID NO:12. Other exemplary muconolactone isomerases include the CatC enzymes from Enterobacter cloacae and Pseudomonas putida described in the following examples, as well as modified versions thereof. Other muconolactone isomerases are known in the art. In some versions of the invention, the one or more modifications in the recombinant microorganisms can comprise a genetic modification that decreases muconolactone isomerase activity. A genetic modification that decreases muconolactone isomerase activity can comprise a genetic modification to a muconolactone isomerase gene. A genetic modification to a muconolactone isomerase gene can comprise a substitution or insertion in or a complete or partial deletion of the muconolactone isomerase gene.

Catechol 2,3-dioxygenase activity is characterized by EC 1.13.11.2 and comprises the ability to catalyze the conversion of catechol and O₂ to 2-hydroxymuconate semialdehyde. Catechol 2,3-dioxygenase activity is performed by catechol 2,3-dioxygenases. An exemplary catechol 2,3-dioxygenase is xylE/XylE (Saro_3857) of Novosphingobium aromaticivorans, the nucleic acid coding sequence of which is SEQ ID NO:13 and the protein sequence of which is SEQ ID NO:14. Other exemplary catechol 2,3-dioxygenases include proteins with catechol 2,3-dioxygenase activity having a sequence at least 80%, at least 85%, at least 90%, at least 95%, or at least 99% identical to SEQ ID NO:14. Other catechol 2,3-dioxygenases are known in the art. In some versions of the invention, the one or more modifications in the recombinant microorganisms can comprise a genetic modification that decreases catechol 2,3-dioxygenase activity. A genetic modification that decreases catechol 2,3-dioxygenase activity can comprise a genetic modification to a catechol 2,3-dioxygenase gene. A genetic modification to a catechol 2,3-dioxygenase gene can comprise a substitution or insertion in or a complete or partial deletion of the catechol 2,3-dioxygenase gene.

Protocatechuate 4,5-dioxygenase activity is characterized by EC 1.13.11.8 and comprises the ability to catalyze the conversion of protocatechuic acid and O₂ to 4-carboxy-2-hydroxy-cis,cis-muconate-6-semialdehyde (CHMS). Protocatechuate 4,5-dioxygenase activity is performed by protocatechuate 4,5-dioxygenases. Exemplary protocatechuate 4,5-dioxygenases are ligAB1/LigAB1 (Saro_2813/2812) and ligAB2/LigAB2 (Saro_1233/1234) of Novosphingobium aromaticivorans. These enzymes are comprised of A subunits (LigA1 or LigA2) and B subunits (LigB1 or LigB2). The nucleic acid coding sequence of the LigA1 subunit is SEQ ID NO:15. The protein sequence of the LigA1 subunit is SEQ ID NO:16. The nucleic acid coding sequence of the LigB1 subunit is SEQ ID NO:17. The protein sequence of the LigB1 subunit is SEQ ID NO:18. The nucleic acid coding sequence of the LigA2 subunit is SEQ ID NO:19. The protein sequence of the LigA2 subunit is SEQ ID NO:20. The nucleic acid coding sequence of the LigB2 subunit is SEQ ID NO:21. The protein sequence of the LigB2 subunit is SEQ ID NO:22. Other exemplary protocatechuate 4,5-dioxygenases include proteins with A and B subunits that together exhibit protocatechuate 4,5-dioxygenase activity and have sequences at least 80%, at least 85%, at least 90%, at least 95%, or at least 99% identical to SEQ ID NOS:16, 18, 20, and/or 22. Other protocatechuate 4,5-dioxygenases are known in the art. In some versions of the invention, the one or more modifications in the recombinant microorganisms can comprise a genetic modification that decreases protocatechuate 4,5-dioxygenase activity. A genetic modification that decreases protocatechuate 4,5-dioxygenase activity can comprise a genetic modification to a protocatechuate 4,5-dioxygenase subunit gene. A genetic modification to a protocatechuate 4,5-dioxygenase subunit gene can comprise a substitution or insertion in or a complete or partial deletion of the protocatechuate 4,5-dioxygenase gene.

“Gene” refers to a nucleic acid sequence capable of producing a gene product and may include such genetic elements as a coding sequence together with any other genetic elements required for transcription and/or translation of the coding sequence. Such genetic elements may include a promoter, an enhancer, and/or a ribosome binding site (RBS), among others. In some versions, multiple genes are configured in an operon, in which multiple coding sequences are operationally connected to a single promoter. Each coding sequence and promoter pair in such instances are considered herein to constitute separate genes, despite comprising the same promoter.

“Gene product” refers to products such as a polypeptide or an mRNA encoded and produced by a particular gene.

“Operationally connected” refers to a relationship between two genetic elements (e.g., a promoter and coding sequence), in which one of the genetic elements controls or affects the activity of the other genetic element.

“Endogenous” used in reference to a genetic element means that the genetic element is native to the microorganism in which it is disposed.

“Exogenous” used in reference to a genetic element means that the genetic element is not native to the microorganism in which it is disposed.

“Heterologous” used in reference to a genetic element means that the genetic element is derived from a different species than that in which it is disposed.

“Recombinant” as used herein with reference to nucleic acid molecules or polypeptides refers to nucleic acid molecules or polypeptides having a non-natural nucleic acid or polypeptide sequence, respectively. Recombinant” as used herein with reference to a gene refers to a gene having a non-natural nucleic acid sequence, is exogenous, is heterologous, or is endogenous to a given microbe but is disposed within the microbe (e.g., within the microbe's genome) at a locus different from the native form of the gene. “Recombinant” as used herein with reference to a cell or microorganism refers to a cell or microorganism that contains a recombinant nucleic acid molecule, polypeptide, or gene.

“Genetic modification” as used herein refers to any difference in the nucleic acid composition of a cell with respect to a corresponding cell, whether in the cell's native chromosome or in endogenous or exogenous non-chromosomal plasmids harbored within the cell.

“Overexpress” as used herein means that a particular gene product is produced at a higher level in one cell, such as a recombinant cell, than in a corresponding cell. For example, a microorganism that includes a recombinant nucleic acid configured to overexpress a gene product produces the gene product at a greater amount than a microorganism of the same species that does not include the recombinant nucleic acid.

A “homologous” gene or protein is a gene or protein inherited in two species from a common ancestor. While homologous genes or proteins can be similar in sequence, similar sequences are not necessarily homologous.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described herein (or other algorithms available to persons of skill) or by visual inspection. For sequence comparison and identity determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is any nucleic acid or amino acid sequence described herein. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)). One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in determining sequence identity of sequences described herein.

In addition to mechanisms described elsewhere herein, genetic modifications for increasing the activity of a gene or protein include but are not limited to placing the coding sequence under the control of a more active promoter, increasing the copy number of genes comprising the coding sequence, introducing a translational enhancer on a gene comprising the coding sequence (see, e.g., Olins et al. Journal of Biological Chemistry, 1989, 264(29):16973-16976), and/or modifying factors (e.g., transcription factors or genes therefor) that control expression of a gene comprising the coding sequence. Increasing the copy number of genes comprising a coding sequence can be performed by introducing one or more additional copies of the native gene to the microorganism, introducing one or more a heterologous homologs to the microorganism, introducing one or more copies of recombinant versions of the native gene or heterologous homolog to the microorganism, etc. Genes expressing a given coding sequence may be incorporated into the microbial genome or included on an extrachromosomal genetic construct such as a plasmid.

In addition to mechanisms described elsewhere herein, genetic modifications for decreasing the activity of a gene or protein include but are not limited to substitutions, partial or complete deletions, insertions, or other variations to a coding sequence or a sequence controlling the transcription or translation of a coding sequence, such as placing a coding sequence under the control of a less active promoter, etc. In some versions, the genetic modifications can include the introduction of constructs that express ribozymes or antisense sequences that target the mRNA of the gene of interest. Various other genetic modifications that decrease the activity of a gene or gene product are described elsewhere herein.

Various methods for introducing genetic modifications are well known in the art and include homologous recombination, among other mechanisms. See, e.g., Green et al., Molecular Cloning: A laboratory manual, 4^{th} ed., Cold Spring Harbor Laboratory Press (2012) and Sambrook et al., Molecular Cloning: A Laboratory Manual, 3^{rd} ed., Cold Spring Harbor Laboratory Press (2001).

The recombinant genes of the invention can be codon-optimized for the particular microorganism in which they are introduced. Codon optimization can be performed for any nucleic acid by a number of programs, including “GENEGPS”-brand expression optimization algorithm by DNA 2.0 (Menlo Park, CA), “GENEOPTIMIZER”-brand gene optimization software by Life Technologies (Grand Island, NY), and “OPTIMUMGENE”-brand gene design system by GenScript (Piscataway, NJ). Other codon optimization programs or services are well known and commercially available.

The recombinant microorganisms of the invention may comprise any type of microorganism. The microorganism may be prokaryotic or eukaryotic. Suitable prokaryotes include bacteria and archaea. Suitable types of bacteria include α- and γ-proteobacteria, gram-positive bacteria, gram-negative bacteria, ungrouped bacteria, phototrophs, lithotrophs, and organotrophs. Suitable eukaryotes include yeast and other fungi. The microorganism in some versions can be from an order selected from the group consisting of Sphingomonadales and Pseudomonadales. The microorganism in some versions can be from a family selected from the group consisting of Sphingomonadaceae, Pseudomonadaceae, and Enterobacteriaceae. The microorganism in some versions can be from a genus selected from the group consisting of Sphingomonas, Sphingobium, Sphingosinicella, Sphingopyxis, Novosphingobium, Pseudomonas, Erythrobacter (e.g., sp. SG61-1L), Altererythrobacter, Enterobacter, and Klebsiella, among others.

The microorganism in some versions can be a phenol-degrading microorganism, such as a phenol-degrading bacterium. Phenol-degrading microorganisms, including phenol-degrading bacteria, are well known in the art. See, e.g., Gu et al. 2016 (Gu Q, Wu Q, Zhang J, Guo W, Wu H, Sun M. Community Analysis and Recovery of Phenol-degrading Bacteria from Drinking Water Biofilters. Front Microbiol. 2016 Apr. 12; 7:495), Ramid-Pujol et al. 2013 (Ramid-Pujol S, Baneras L, Artigas J, Romani A M. Changes of the phenol-degrading bacterial community during the decomposition of submersed Platanus acerifolia leaves. FEMS Microbiol Lett. 2013 January; 338(2):184-91), Bastos et al. 2000 (Bastos A E, Moon D H, Rossi A, Trevors J T, Tsai S M. Salt-tolerant phenol-degrading microorganisms isolated from Amazonian soil samples. Arch Microbiol. 2000 November; 174(5):346-52), van Schie et al. 1998 (van Schie P M, Young L Y. Isolation and characterization of phenol-degrading denitrifying bacteria. Appl Environ Microbiol. 1998 July; 64(7):2432-8), Paisio et al. 2012 (Paisio C E, Talano M A, Gonzilez P S, Busto V D, Talou J R, Agostini E. Isolation and characterization of a Rhodococcus strain with phenol-degrading ability and its potential use for tannery effluent biotreatment. Environ Sci Pollut Res Int. 2012 September; 19(8):3430-9), Kumari et al. 2013 (Kumari S, Chetty D, Ramdhani N, Bux F. Phenol degrading ability of Rhodococcus pyrinidivorans and Pseudomonas aeruginosa isolated from activated sludge plants in South Africa. J Environ Sci Health A Tox Hazard Subst Environ Eng. 2013; 48(8):947-53), among others. Examples of phenol-degrading microorganisms include Pseudomonas putida (Abu Hamed T., Bayraktar E., Mehmetoglu U., Mehmetoglu T. (2004). The biodegradation of benzene, toluene and phenol in a two-phase system. Biochem. Eng. J. 19 137-146), Gliomastix indicus (Singh R. K., Kumar S., Kumar S., Kumar A. (2008) Biodegradation kinetic studies for the removal of p-cresol from wastewater using Gliomastix indicus MTCC 3869. Biochem. Eng. J. 40 293-303), Sphingomonas chlorophenolica (Nair C. I., Jayachandran K., Shashidhar S. (2008). Biodegradation of phenol. Afr. J. Biotechnol. 7 4951-4958), Bacillus brevis (Arutchelvan V., Kanakasabai V., Elangovan R., Nagarajan S., Muralikrishnan V. (2006). Kinetics of high strength phenol degradation using Bacillus brevis. J. Hazardous Materials 129 216-222), and Cyanobacterium synechococcus (Song H., Liu Y., Xu W., Zeng G., Aibibu N., Xu L., et al. (2009). Simultaneous Cr (VI) reduction and phenol degradation in pure cultures of Pseudomonas aeruginosa CCTCC AB91095. Bioresour. Technol. 100 5079-5084), and Acinetobacter sp. (Gu Q, Wu Q, Zhang J, Guo W, Wu H, Sun M. Community Analysis and Recovery of Phenol-degrading Bacteria from Drinking Water Biofilters. Front Microbiol. 2016 Apr. 12; 7:495). Other examples of phenol-degrading microorganisms include Achromobacter sp., Alcaligenes denitrificans, Arthrobacter sp., Arthrobacter sulphureus, Acidovorax delafieldii, Bacillus cereus, Brevibacterium sp., Burkholderia sp., Burkholderia cepacia, Burkholderia cocovenenans, Burkholderia xenovorans, Chryseobacterium sp., Cycloclasticus sp., Janibacter sp., Marinobacter, Mycobacterium sp., Mycobacterium flavescens, Mycobacterium vanbaalenii, Mycobacterium sp., Nocardioides aromaticivorans, Pasteurella sp., Polaromonas naphthalenivorans, Pseudomonas sp., Pseudomonas paucimobilis, Pseudomonas vesicularis, Pseudomonas putida, Pseudomonas fluorescens, Pseudomonas stutzeri, Pseudomonas saccharophilia, Ralstonia sp., Rhodococcus sp., Rhodococcus erythropolis, Staphylococcus sp., Stenotrophomonas maltophilia, Sphingomonas yanoikuyae, Sphingomonas sp., Sphingomonas paucimobilis, Sphingomonas wittichii, Terrabacter sp., and Xanthamonas sp. (Seo J-S, Keum Y-S, Li Q X. Bacterial Degradation of Aromatic Compounds. International Journal of Environmental Research and Public Health. 2009; 6(1):278-309.) Other examples of phenol-degrading microorganism include Acinetobacter calcoaceticus, Rhodococcus aetherivorans, Rhodococcus ruber SD3, Aspergillus oryzae, and Aspergillus flavus (Xu N, Qiu C, Yang Q, Zhang Y, Wang M, Ye C, Guo M. Analysis of Phenol Biodegradation in Antibiotic and Heavy Metal Resistant Acinetobacter lwoffii NL1. Front Microbiol. 2021 Sep. 10; 12:725755), among others.

An exemplary microorganism from the genus Novosphingobium is Novosphingobium aromaticivorans. Novosphingobium aromaticivorans DSM12444 can naturally catabolize multiple aromatic compounds containing H, G, and S units via protocatechuic acid.

The recombinant microorganisms are preferably configured to exhibit enhanced production of cis,cis-muconic acid with respect to a corresponding microorganism. “Production” in this context refers to the extracellular appearance of cis,cis-muconic acid in media in which the recombinant microorganism are cultured. The recombinant microorganisms in such versions may include any one or more of the modifications described herein, in any combination.

The recombinant microorganisms of the invention preferably exhibit enhanced cis,cis-muconic acid production with respect to the corresponding microorganism when the recombinant microorganism and the corresponding organism are grown under identical conditions. The cis,cis-muconic acid production may be enhanced by a factor of at least about 1.1, at least about 1.5, at least about 2, at least about 2.5, at least about 3, at least about 3.5, at least about 4, at least about 4.5, at least about 5, at least about 5.5, at least about 6, or at least about 6.5 and/or up to about 6.5, up to about 7, or more. Such increases may reflect an increase by mass.

The cis,cis-muconic can be produced by culturing a recombinant microorganism of the invention in a medium comprising a plant-derived phenolic. The plant-derived phenolic can comprise any of a number of phenolics obtained from processing plant lignocellulosic biomass. Exemplary plant-derived phenolics comprise syringyl phenolics, guaiacyl phenolics, and p-hydroxyphenyl phenolics. Exemplary syringyl phenolics include syringaldehyde, syringic acid, and S-diketone. Exemplary guaiacyl phenolics include vanillin, vanillic acid, and G-diketone. Exemplary hydroxyphenyl phenolics include p-coumaric acid, p-hydroxybenzaldehyde, and p-hydroxybenzoic acid. Other plant-derived phenolics include methyl guaiacol, propyl guaiacol, dihydroconiferyl alcohol, methyl syringol, p-hydroxy benzoic acid methyl ester, dihydrop-hydroxy cinnamic acid methyl ester, dihydrosyringol alcohol, and dihydroferulic acid methyl ester, among others.

The plant-derived phenolic can be derived and/or provided in the form of depolymerized lignin, such as chemically depolymerized lignin. Methods of depolymerizing lignin are well known in the art. See Pandey et al. 2010 (Pandey M P, Kim C S. Lignin Depolymerization and Conversion: A Review of Thermochemical Methods. Chemical & Engineering Technology, 2010, Vol. 34, Issue 1, pp. 3-145) and Wang et al. 2013 (Wang H, Tucker M, Ji Y. Recent Development in Chemical Depolymerization of Lignin: A Review. Journal of Applied Chemistry, 2013, Volume 2013, Article ID 838645).

The depolymerized lignin can be derived from pretreated lignocellulosic biomass. Methods of pretreating lignocellulosic biomass are well known in the art. See Kumar et al. 2017 (Kumar AK and Sharma S. Recent Updates on Different Methods of Pretreatment of Lignocellulosic Feedstocks: A Review. Bioresour. Bioprocess. (2017) 4:7); Kumar et al. 2009 (Kumar, P.; Barrett, D. M.; Delwiche, M. J.; Stroeve, P., Methods for Pretreatment of lignocellulosic Biomass for Efficient Hydrolysis and Biofuel Production. Industrial & Engineering Chemistry Research 2009, 48, (8), 3713-3729); Wang et al. 2013 (Wang H, Tucker M, Ji Y. Recent Development in Chemical Depolymerization of Lignin: A Review. (2013) Journal of Applied Chemistry. 2013:1-9), and Karlen et al. 2020 (Karlen S D, Fasahati P, Mazaheri M, Serate J, Smith R A, Sirobhushanam S, Chen M, Tymkhin V I, Cass C L, Liu S, Padmakshan D, Xie D, Zhang Y, McGee M A, Russell J D, Coon J J, Kaeppler H F, de Leon N, Maravelias C T, Runge T M, Kaeppler S M, Sedbrook J C, Ralph J. Assessing the viability of recovering hydroxycinnamic acids from lignocellulosic biorefinery alkaline pretreatment waste streams. ChemSusChem. 2020 Jan. 26). Examples include chipping, grinding, milling, steam pretreatment, ammonia fiber expansion (AFEX, also referred to as ammonia fiber explosion), ammonia recycle percolation (ARP), CO₂ explosion, steam explosion, ozonolysis, wet oxidation, acid hydrolysis, dilute-acid hydrolysis, alkaline hydrolysis, organosolv, ionic liquids, gamma-valerolactone, and pulsed electrical field treatment, among others.

The lignocellulosic biomass can be derived from any source, such as corn cobs, corn stover, cotton seed hairs, grasses, hardwood stems, leaves, newspaper, nut shells, paper, softwood stems, sorghum, switchgrass, waste papers from chemical pulps, wheat straw, wood, woody residues, mixed biomass species such as those produced by native prairie, and other sources.

The medium in some versions can additionally or alternatively comprise a fermentable sugar. Non-limiting examples of suitable fermentable sugars include adonitol, arabinose, arabitol, ascorbic acid, chitin, cellubiose, dulcitol, erythrulose, fructose, fucose, galactose, glucose, gluconate, inositol, lactose, lactulose, lyxose, maltitol, maltose, maltotriose, mannitol, mannose, melezitose, melibiose, palatinose, pentaerythritol, raffinose, rhamnose, ribose, sorbitol, sorbose, starch, sucrose, trehalose, xylitol, xylose, and hydrates thereof, among others.

In some versions, the fermentable sugar may be replaced by other organic compounds that support growth of the recombinant microorganism. This includes but is not limited to the other organic compounds that are present in the deconstructed biomass fractions from the crops or plant species mentioned above.

A recitation herein of a microorganism “comprising” a mutation in or to a particular gene refers to a gene that would be present were it not for the mutation, e.g., the gene present in a corresponding microorganism. Thus, the recitation of a microorganism “comprising” a mutation in or to a particular gene encompasses a mutated form of the gene present in the microorganism, a partially deleted remnant of the gene present in the microorganism, a complete absence of the gene (e.g., as resulting from a complete deletion of the gene) in the microorganism, or other configurations.

The methods can further comprise isolating cis,cis-muconic acid from the recombinant microorganism and/or the medium. Methods of isolating cis,cis-muconic acid from a medium are provided in the attached examples and otherwise known in the art.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### Summary

The transition to production of commodity chemicals from renewable resources is an important goal towards increasing the environmental and economic sustainability of industrial processes. The aromatics in plant biomass are an underutilized and abundant renewable resource for the production of valuable chemicals. However, due to the chemical composition of plant biomass, many deconstruction methods generate a heterogenous mixture of aromatics, thus making it difficult to extract valuable chemicals using current methods. Therefore, recent efforts have focused on harnessing the native or engineered pathways of microorganisms to convert a diverse set of aromatics into a single product. Novosphingobium aromaticivorans DSM12444 has the native ability to metabolize a wide range of aromatics and thus is a potential chassis for conversion of these abundant compounds to commodity chemicals.

The platform chemical cis,cis-muconic acid (ccMA) provides facile access to a number of monomers used in the synthesis of commercial plastics. It is also a metabolic intermediate in the β-ketoadipic acid pathway of many bacteria and, therefore, a current target for microbial production from abundant renewable resources via metabolic engineering. This study investigates Novosphingobium aromaticivorans DSM12444 as a chassis for the production of ccMA from biomass aromatics. We show that the N. aromaticivorans genome encodes a previously uncharacterized PCA decarboxylase and a catechol 1,2-dioxygenase, which can be used for the conversion of aromatic metabolic intermediates to ccMA. This study confirmed the activity of these 2 enzymes in vitro. From these results, we generated one strain that is completely derived from native genes and another strain that contains heterologous genes. Both of these strains exhibited stoichiometric production of ccMA from PCA and produced greater than 100% yield of ccMA from the aromatic monomers that were identified in liquor derived from alkaline pretreated biomass. Our results show that a strain completely derived from native genes is comparable in producing ccMA from biomass aromatics to strains that are derived from heterologous genes. Overall, this work highlights the capacity of N. aromaticivorans as a chassis for ccMA production from biomass.

This study demonstrates that metabolic engineering of N. aromaticivorans can be used to produce the commodity chemical cis,cis-muconic acid (ccMA) from renewable and abundant biomass aromatics.

### Introduction

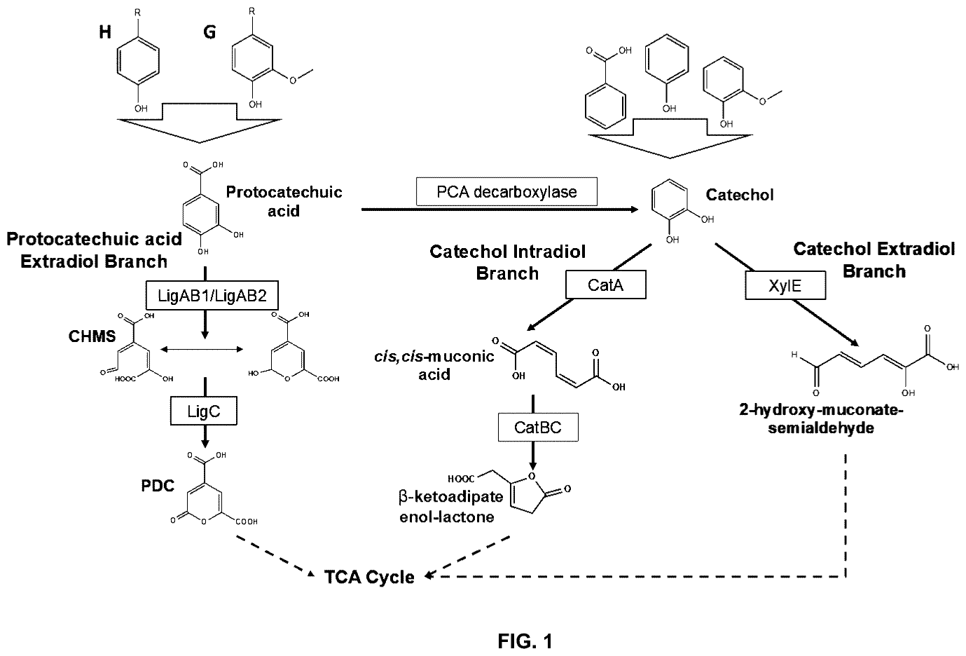

Strategies for biological ccMA production from aromatics typically relies on the intradiol cleavage of catechol by a catechol 1,2-dioxygenase, CatA. Catechol is a known intermediate in the aromatic metabolism of benzoic acid, guaiacol and phenol (FIG. 1) (17, 18). Pathways for production of ccMA from biomass-derived H- and G-type aromatics have been designed to divert the pathway intermediate protocatechuic acid (PCA) to catechol often through heterologous expression of a PCA decarboxylase that is native to either Klebsiella pneumoniae or Enterobacter cloacae (5, 6, 19). Thus far, Pseudomonas putida KT2440 is the best described bacterial host strain for ccMA production from either pure aromatics (p-coumaric or ferulic acids) or deconstructed biomass (20). Engineering of P. putida KT2440 for ccMA production required the use of exogenous promoters for expression of foreign genes and deletion of a gene that encodes a global catabolic transcriptional regulator (21, 22) to overcome metabolic bottlenecks that included accumulation of several pathway intermediates (PCA, vanillin and 4-hydroxybenzoic acid) (6, 23, 24). Therefore, we sought to expand the scope of bacterial hosts for producing ccMA from biomass aromatics by leveraging the native metabolic pathways of the aromatic metabolizing bacterium Novosphingobium aromaticivorans DSM12444.

N. aromaticivorans is one of several sphingomonads that are being studied as a potential chassis for production of chemicals from biomass aromatics (4, 25). This α-protobacterium, isolated from a polyaromatic hydrocarbon-contaminated site, can utilize many aromatics as a sole carbon source (26-28). N. aromaticivorans and other sphingomonads have the native ability to cleave major inter-subunit linkages of lignin aromatic oligomers making these microbes ideal for converting renewable sources of mixed aromatics into products (11, 29-32). The genetic tractability of N. aromaticivorans has enabled the engineering of mutant strains that can stoichiometrically produce the commodity chemical 2-pyrone-4,6-dicarboxylic acid (PDC) from native G-, H-, and S-aromatics or aromatic diketones that are generated during lignin deconstruction (31-33). These characteristics of N. aromaticivorans make it attractive for microbial funneling of the heterogeneous mixture of aromatics in deconstructed biomass. However, knowledge gaps still remain on the number and diversity of enzymes that compose this bacterium's aromatic metabolic pathways. This work sought to address some of these knowledge gaps by investigating the ability of N. aromaticivorans to serve as a host for ccMA production from biomass aromatics.

Here, we evaluated the potential of engineering N. aromaticivorans as a chassis for ccMA production from lignin biomass. We determined possible PCA decarboxylase and CatA enzymes in the genome of N. aromaticivorans. These enzymes are both important for the diversion of PCA to catechol and subsequent production of ccMA (FIG. 1). We used in vitro assays to confirm the activity of these putative enzymes and applied this information to generate defined mutants that test the predicted pathway for conversion of PCA to ccMA by N. aromaticivorans. Through the combination of these in vitro assays and the analysis of defined mutants, we were able to identify a previously unreported PCA catabolic pathway in N. aromaticivorans. This new information makes N. aromaticivorans unique in comparison to other reported ccMA production hosts which do not possess a native decarboxylase for conversion of PCA to catechol (20). We then explored whether the expression of these native genes could compete with expression of heterologous genes used previously for ccMA production by placing individual genes under control of native aromatic-inducible promoters in the genome. Overall, this study revealed the ability to engineer N. aromaticivorans to stoichiometrically convert PCA to ccMA and to produce greater than 100% yields of ccMA from the measured aromatics in biomass alkaline pretreatment liquor (APL). It also highlights that an engineered N. aromaticivorans strain completely derived from native genes produces ccMA at a similar rate and production yield as seen with a N. aromaticivorans strain that heterologously expresses the enzymes from E. cloacae (4-6, 34). This study illustrates the tractability and scope of N. aromaticivorans aromatic metabolism and the ability to use this host for production of ccMA as a valuable product from biomass-derived aromatics.

### Materials and Methods

### Genes and Sequences

Genes and the sequences of the genes used in this study are provided in Tables 1 and 2.

TABLE 1
N. aromaticivorans genes used in this
study with associated locus tags.

| Gene | Organism | Locus tag |
|---|---|---|
| ligAB1 | Novosphingobium aromativicorans | Saro_2812-13 |
| | DSM 12444 | |
| ligAB2 | Novosphingobium aromativicorans | Saro_1233-34 |
| | DSM 12444 | |
| nadB | Novosphingobium aromativicorans | Saro_3873 |
| | DSM 12444 | |
| nadC | Novosphingobium aromativicorans | Saro_3877 |
| | DSM 12444 | |
| nadD | Novosphingobium aromativicorans | Saro_3878 |
| | DSM 12444 | |
| ecdB | Enterobacter cloacae | ECL_04083 |
| ecaroY | Enterobacter cloacae | ECL_01944 |
| ecdD | Enterobacter cloacae | ECL_04081 |
| nacatA | Novosphingobium aromativicorans | Saro_3830 |
| | DSM 12444 | |
| eccatA | Enterobacter cloacae | HWQ17_22740 |
| naxylE | Novosphingobium aromativicorans | Saro_3857 |
| | DSM 12444 | |

TABLE 2
DNA sequences of genes inserted into the genome of N. aromaticivorans.
Start and stop codons are shown in bold. Coding sequences are indicated
by upper case and relevant ribosomal binding sites are shown in lower case.
In the EcDec or NaDec decarboxylase gene clusters, sequences in underline
denote the prenyltransferase coding region, those in underlined italics
denote the PCA decarboxylase coding region and those in italics represent
the coding sequence for a gene of unknown function.

| Gene | Sequence |
|---|---|
| NaCatA | ATGCCTGCCACCTTCGCCAGTTCCGATTCCGTGCAGAAGCTCTTCGATC |
| | GCGCCTGCGGTCTTGATTGCGCAGGCGGCAATCCCCGCCTCAAGGCGAT |
| | CATGCGCGACCTTCTCCAGGCAACGGCCGACATCATCGTCAAGCATGAC |
| | GTGTCCGAAAGCGAGTTCTGGCAGGCGACCCGCTATCTTGCCGATGGCG |
| | CCGGCGAGATCGGCCTGATCGTCCCCGGCATCGGCCTCGAACACTTCCT |
| | CGATCTCTACATGGACGCCAAGGACGCCGAAGCCGGCCTCACCGGCGG |
| | AACCCCGCGCACGATCGAAGGCCCGCTCTACGTCGCTGGTGCACCGCTG |
| | GTGGATGGCAGTGACGAAGTGGACCTGACTTCCGACCCCGACGATACC |
| | GACACGCTGCACATGACCGGCACGATCACCGGCCCCGATGGCGAGCCG |
| | GTCAAGGACGCGATCCTCCACGTCTGGCACGCGAACAGCAAGGGCTGG |
| | TATTCGCACTTCGATCCCACGAGCGAGCAGACCCCGTTCAACAACCGCC |
| | GCCGCATCCGCGTCCCCGCCGACGGTCGCTACGCCTTCCGCTCCAAGAT |
| | GCCGCATGGCTATTCCGTGCCGCCGGGTGGCGCCACCGACGTGCTGATG |
| | CAGGCGCTCGGCCGCCACGGCAATCGCCCAGCGCACGTCCACTTCTTCG |
| | TCGAGGCGCCGGGCTACCGCACGCTGACCACGCAGATCAACTTCGGCG |
| | ACGACCCCTTCGCGGCCGACGATTTCGCCTTCGGCACGCGAGAGGGCTT |
| | GCTGCCGGTGCCGAGCCGCCAGGGCGATACCGCCCACATCGCGTTCGA |
| | CTTCCAGCTCCAGCGCGCCCGCTCGGAGGACGAGCAGCGGTTCTCGGA |
| | ACGCACCCGCGCCCAGGCCTGA (SEQ ID NO: 7) |
| EcCatA | ATGTCGAAGAACCCGTCGCAGCAGTCGGAACTCGAAACCCTCCTCGCC |
| | ATCTCGTCGGGCCTCAACACCGACGGCGGCAACGAACGCGTCAAGCGC |
| | GTCATCCACCAGCTCCTCAACGACCTCTGCCACACCATCAAGACCTTCG |
| | ACATCTCGGACGAAGAATTCTGGATCGCCGTCAACTACCTCAACGAACT |
| | CGGCGAACGCAAGGAAGCCGCCCTCCTCGCCGCCGGCCTCGGCCTCGA |
| | ACACTACCTCGACATGCGCGCCGACGAAAAGGAAGCCGCCTCGGTCTC |
| | GCGCGCCGGCACCCCGCGCACCATCGAAGGCCCGCTCTACGTCGCCAA |
| | CGCCCCGCTCTCGGACCACTTCGCCCGCATGGACGACGGCTCGGAACGC |
| | GCCGAAGCCATGTGGCTCCACGGCACCGTCACCGACATCGACGGCAAG |
| | CCGGTCGCCGGCGCCATCGTCGACATCTGGCACGCCAACACCCACGGC |
| | GGCTACTCGTTCTTCGACCCGTCGCAGTCGGAATACAACCTCCGCCGCC |
| | GCGTCAAGACCGCCGCCGACGGCTCGTACGCCGTCCGCTCGATCGTCCC |
| | GTGCGGCTACGGCTGCCCGCCGGACGGCCCGACCCAGAAGCTCCTCAA |
| | CGAACTCGGCCGCCACGGCAACCGCCCGGCCCACATCCACTTCTTCGTC |
| | TCGGCCCCGGGCTTCAAGCACCTCACCACCCAGATCAACCTCAACGGCG |
| | ACCGCTACCTCTGGGACGACTTCGCCTTCGCCACCCGCGAAGAACTCAT |
| | CGCCGACCCGGTCAAGGTCACCGACTCGACCCTCGCCCGCGAACGCGA |
| | CATCCACGAACCGCACACCGAAGTCTCGTTCTCGTTCACCCTCGTCAAG |
| | GCCGCCGGCGCCGAAGAAGAAGCCCGCGGCAAGCGCGCCCGCGTCAAG |
| | GAATGA (SEQ ID NO: 27) |
| NaDec | GTGAAGCGCATGGTCGTGGGGATTACCGGCGCAACCGGCTCGGTCTAT |
| | GGTCTTCGCCTGCTTGAGCTGCTGCGCGAGACGGGCGGTTGGGAAACCC |
| | ATCTGGTAATGTCTCCGGCTGCGCTGCTCAACATTCGCGAGGAACTGCC |
| | CGAAGGCAAAGCCCGGCTCGAAGCGCTGGCCGATGTGGTGCACAACGT |
| | CCGCAACGTCGGCGCCTCGATCGCCAGCGGTTCGTTCGTATGCGAAGGC |
| | ATGGCGATTGCGCCCTGTTCGATGCGCACGCTGGGCGCGGTGGCGCACG |
| | CCCTGTCCGACAACCTTATCACCCGCGCGGCCGACGTGATGCTGAAGGA |
| | ACGGCGCCGCCTGGTGATGATCACCCGCGAAGCGCCGCTCAACCTGGC |
| | GCACCTGCGCAACATGACGGCCTGCACCGAAATGGGGGCGGTGATCTT |
| | CCCCCCGGTGCCGGCCTTCTATGCGCGGCCGACCTCGCTGGCCGACGTG |
| | GTCGATCACACCTGCATGCGGGTACTGGATCTGTTCGGGCTTCATGCGA |
| | AGTCGGAGAAACGCTGGCAAGGCCTTAGCAAAGAGGCGGCAAGCCTTG |
| | TTCCGGGTGCTGGGCAAATGGAAGGGAATTGAgaATGACCATGAACGATC |
| | TCCCTAACCGCGCCCGCTCGATCTCGTCGCTGCGCGACTTCCTCGAACTGC |
| | TCGAGGATGCCGGCCAGGCGATCACCTGGAGCGATGCGGTGATGCCCGAA |
| | CCCGGCGTGCGCAACATAGCCGTCGCCGCATCGCGCGATGCCAACGGCGC |
| | GCCGGCGATCGTATTCGACAATATCACCGGTTACCCCGGCAAGCGCTTGGC |
| | GGTGGGCGTCCATGGTTCGTGGGACAACATCGCCCTGCTGCTGGGCCGAC |
| | CTAAAGGCACGACCATCCGCGAGCTTTTCTTCGAGATCGCCGGCCGCTGGG |
| | GCGATCAGGAAGCGCAAATCAGCTTTGTCCCAGAAGCCCAGGCCCCGGTGC |
| | ACGAATGCCGGATCGAACAGGACATCAACCTTTACGATGTCCTGCCGGTCTA |
| | TCGGATCAACGAATACGATGGCGGGTTCTACATCGGCAAGGCCTCGGTCGC |
| | CTCGCGCGATCCGCTCGATCCAGACAATTTCGGCAAGCAGAATGTCGGCAT |
| | CTATCGCCTGCAGATCCAGGGGCCGGACACCTTCACCCTGATGACGATCCC |
| | CTCCCACGACATGGGACGTCAGATCATGGCGGCCGAACGGGAAGGCGTTCC |
| | GCTAAAGATTGCGGTCATGCTGGGTAATCATCCCGGCCTTGCGGTGTTTGCT |
| | GCCACCCCGATCGGCTACGAGGAATCGGAATATTCCTATGCCTCGGCGATG |
| | ATGGGCGCGCCAATCCGGCTGACCAAATCGGGCAACGGGATCGACATCCTG |
| | GCCGACAGCGAAATCGTGATAGAGGCCGAACTGCAACCGGGTGGACGCGA |
| | GCTGGAAGGGCCGTTCGGCGAATTCCCCGGTTCCTACAGCGGCGTGCGCAA |
| | GGCGCCGATCTTCAAGGTCACGGCGGTGTCGCACCGGCGCGATCCGATCTT |
| | CGAGAACATTTACATCGGGCGCGGCTGGACCGAGCACGATACGCTGATCGG |
| | CCTGCACACCTCCGCCCCGATCTATGCCCAGCTGCGCCAGAGCTTCCCCGA |
| | AGTCACCGCGGTCAACGCGCTTTACCAGCACGGACTGACCGGGATCATCTC |
| | GGTCAAAAACCGCATGGCCGGCTTTGCCAAGACGGTCGCGCTGCGCGCGCT |
| | GAGCACGCCGCACGGCGTGATGTACCTCAAGAACCTGATTATGGTCGATGC |
| | CGATGTCGATCCGTTCGATCTCAACCAAGTGATGTGGGCGCTTTCGACCCGC |
| | ACCCGTGCGGACGATATCATCGTGCTGCCCAACATGCCTGCCGTGCCGATC |
| | GATCCTTCGGCAGTGGTCCCGGGCAAGGGGCACCGCCTGATCATCGACGC |
| | GACCAGCTATCTCCCGCCCGATCCGGTGGGTGAAGCGCACCTTGTCACCCC |
| | GCCGACCGGGGACGAGATCGACGCCCTGAGCAAGCGGATCCGCGAAATGC |
| | AGCTGGGAGCCCTGTCATGACCACCACCGTCTGCGGGCGCTGCAAATCGA |
| | GCGGCGCTGTCACCGATCATCAGGGCAGGCAGGACGGCGCGGTCGTGTGG |
| | ACGATCCTGCGCTGCCCGACCTGCAACTTTTCCTGGCGCGACAGCGAACCG |
| | GCCCGCGCTATCGACCCGGCTGTGCGCTCGGCCGATTTCGCCGTCGATGTC |
| | GGCGATCTCCAGCGTTATCCCAAGATTCTCCAGCAATAA (SEQ ID NO: 28) |
| EcDec | ATGAGGCTCATCGTGGGCATGACGGGAGCCACGGGCGCTCCGCTTGGC |
| | GTGGCCCTCCTGCAGGCGCTCCGCGACATGCCCGAGGTTGAAACCCATC |
| | TGGTGATGTCGAAGTGGGCGAAGACCACCATCGAGCTGGAAACGCCGT |
| | ATACCGCGCAGGACGTCGCTGCCCTGGCCGACGTCGTCCACAGCCCTGC |
| | CGATCAGGCAGCCACCATCTCGTCGGGCTCGTTCCGCACCGATGGCATG |
| | ATCGTCATTCCCTGCAGCATGAAGACGCTTGCAGGCATTCGCGCGGGCT |
| | ATGCCGAAGGGCTTGTCGGTCGTGCGGCAGATGTTGTGCTGAAAGAAG |
| | GTCGCAAGCTGGTGCTGGTCCCGCGCGAAACGCCGCTCAGCACCATCCA |
| | TCTGGAGAACATGCTCGCGCTTTCCCGCATGGGGGTGGCGATGGTGCCG |
| | CCCATGCCCGCGTACTACAACCATCCGCAAACCGCCGACGACATCACCC |
| | AGCACATCGTGACCCGCGTCCTCGACCAGTTCGGTCTGGAGCACAAGA |
| | AGGCACGTCGCTGGAATGGCCTGCAGGCGGCGAAGCACTTCAGCCAGG |
| | AGAACAACGACGGCATCTGAtgctgggcaaatggaagggaattgagaATGCAGAACCC |
| | CATCAACGACCTCCGCTCTGCCATCGCGCTGCTGCAACGCCATCCCGGTCA |
| | CTATATCGAAACCGACCACCCGGTCGATCCCAATGCTGAACTGGCGGGCGT |
| | CTATCGCCATATCGGCGCGGGCGGTACCGTCAAACGCCCCACCCGCACGG |
| | GCCCGGCCATGATGTTCAACAGCGTGAAGGGCTACCCTGGCTCCCGCATCC |
| | TGGTCGGTATGCATGCCAGCCGGGAACGCGCGGCGCTTCTGCTGGGCTGT |
| | GTCCCCTCGAAGCTGGCACAGCACGTCGGTCAGGCGGTGAAGAACCCGGTT |
| | GCACCGGTGGTGGTTCCAGCCTCGCAGGCACCGTGCCAGGAGCAGGTCTT |
| | CTATGCCGACGATCCGGACTTCGACCTGCGTAAGCTGCTTCCGGCCCCGAC |
| | CAACACGCCGATTGATGCAGGCCCGTTCTTCTGCCTGGGGCTGGTCCTGGC |
| | AAGCGATCCGGAAGACACCTCGCTGACCGATGTGACCATTCACCGTCTCTGC |
| | GTGCAGGAGCGAGACGAACTCTCGATGTTCCTTGCCGCCGGCCGCCATATC |
| | GAAGTCTTTCGCAAGAAGGCCGAAGCGGCGGGCAAACCGCTGCCGGTCAC |
| | CATCAACATGGGACTTGACCCGGCTATCTACATAGGGGCCTGCTTCGAAGCG |
| | CCCACCACGCCCTTCGGTTACAACGAGCTTGGCGTTGCCGGGGCACTCCGC |
| | CAGCAACCGGTGGAGCTGGTCCAGGGCGTAGCGGTCAAGGAGAAAGCGAT |
| | CGCGCGGGCGGAAATCATCATCGAGGGCGAACTGCTTCCCGGCGTGCGCG |
| | TCCGCGAAGATCAGCACACCAACACCGGCCACGCCATGCCGGAGTTCCCGG |
| | GCTACTGCGGCGAGGCGAATCCGTCGCTGCCGGTGATCAAGGTGAAAGCC |
| | GTGACGATGCGAAACCATGCGATCCTGCAGACGCTGGTGGGCCCTGGCGAA |
| | GAGCACACCACGCTTGCCGGTCTGCCGACCGAGGCCAGCATTCGCAACGC |
| | GGTCGAAGAGGCCATTCCCGGCTTTCTGCAGAACGTCTACGCCCACACCGC |
| | CGGAGGCGGTAAGTTCCTCGGCATCCTACAGGTGAAGAAGCGCCAGCCGTC |
| | GGACGAAGGACGTCAGGGCCAGGCGGCACTCATCGCCCTGGCCACCTATTC |
| | CGAGCTGAAGAACATCATCCTCGTGGACGAAGACGTGGACATCTTCGACAG |
| | CGACGACATCCTGTGGGCAATGACCACCCGCATGCAGGGCGATGTGAGCAT |
| | CACCACGCTTCCGGGGATCCGCGGCCACCAGCTGGATCCGTCGCAGTCGC |
| | CGGACTACAGCACCTCGATCCGTGGAAACGGCATCTCCTGCAAGACTATCTT |
| | CGACTGCACGGTGCCGTGGGCGCTGAAGGCGCGGTTCGAACGGGCGCCGT |
| | TCATGGAGGTCGACCCCACACCGTGGGCGCCGGAGCTGTTCAGCGACAAGA |
| | AGTGAcagctgggagccctgtcATGATCTGCCCGCGCTGCGCCGACGAGCAGATCG |
| | AGGTCATGGCCACCAGCCCGGTGAAGGGCATCTGGACCGTCTACCAGTGCC |
| | AGCACTGCCTGTACACCTGGCGGGACACCGAACCGCTTCGTCGCACCTCGC |
| | GCGAGCACTATCCCGAAGCGTTCCGCATGACGCAGAAGGACATCGATGAAG |
| | CGCCGCAGGTGCCCACGATTCCTCCGCTCCTGTGAgctgaccagacaggagtagtaccc |
| | (SEQ ID NO: 29) |

### Bacterial Strains, Growth Media, and Culturing Conditions

N. aromaticivorans 12444A1879 (31) (called 12444 in Table 3) is a derivative of wild-type strain DSM 12444 in which a putative sacB gene (Saro_1879) was deleted to create a strain amenable to genomic modifications using a variant of the pK18mobsacB plasmid (59) that contains both kanamycin resistance and sacB. Plasmids for cloning were constructed with the NEBuilder HiFi DNA Assembly Master Mix (New England Biolabs; Ipswich, MA), as described in the supplementary material SI. A complete list of the mutant strains and a list of the primers used to generate these mutant strains is shown in Table 3 and Table 4.

TABLE 3
N. aromaticivorans mutant strains used in this study.

| Strain name | Genotype | Source |
|---|---|---|
| 12444 | 12444Δ1879 | Perez et al. 2019 (31) |
| 12444_ΔligAB1/2 | 12444Δ1879ΔligABΔligAB2 | Perez et al. 2021 (32) |
| 12444_PCA | ΔligAB1/2 ΔSaro3873-3878 | This study |
| LigAB1_EcDec | 12444_PCA ΔligAB1:EcdB/EcaroY/EcdD | This study |
| LigAB1_NaDec | 12444_PCA ΔligAB1:NadBCD | This study |
| EcDec_cat | LigAB1_EcDec ΔxylE ΔcatBC | This study |
| NaDec_cat | LigABI_NaDec ΔxylE ΔcatBC | This study |
| EcDec_ccMA | EcDec_cat ΔxylE: EccatA | This study |
| NaDec_ccMA | EcDec_cat ΔxylE: NacatA | This study |

TABLE 4
Primers used for deletion and gene insertions in N. aromaticivorans. All Primers are
shown in the 5′ to 3′ orientation.

| Genomic | PCR | | |
|---|---|---|---|
| Modification | Reaction | Primers | Notes |
| Deletion of | Amplify | Saro_3877-8 Ampl F pk18 HiFi | Bold and bold |
| Saro_3877-8 | upstream of | CGATTCATTAATGCAGCTGGCACG | underline |
| | Saro_3877 | ACAGCGATCTGCTCGCTAAATTGTG | sequences are |
| | | GAAGGAG (SEQ ID NO: 30) | complementary to |
| | | Saro_3877-8 HiFi Del R | the end regions of |
| | | GCTGGAGAATCTTGGGATAACCGTTCC | linearized |
| | | CGCCTGTCAGTGTC (SEQ ID NO: 31) | pK18mobsacB. |
| | Amplify | Saro_3877-8 HiFi Del F | Regions in italics |
| | downstream | CTGACAGGCGGGAACGGTTATCCCAA | or underlined |
| | of Saro_3878 | GATTCTCCAGCAATAAGGCTC (SEQ | italics are |
| | | ID NO: 32) | complementary to |
| | | Saro_3877-8 Ampl R pk18 HiFi | each other. |
| | | GTTTCTGCGGACTGGCTTTCTAGA | |
| | | TGTTCCGTAAAGGTTCCAGTAGCCT | |
| | | AGTCCG (SEQ ID NO: 33) | |
| Deletion of | Amplify | Saro3873-8 pk18 HiFi Ampl-F | Bold and bold |
| Saro_3873-8 | upstream of | CGATTCATTAATGCAGCTGGCACG | underline |
| | Saro_3873 | ACAGGATGTTACGAAGTTCACCTTC | sequences are |
| | | ACCGGTTC (SEQ ID NO: 34) | complementary to |
| | | Saro3873-8 HiFi Del R | the end regions of |
| | | CTGGAGAATCTTGGGATAACCAGGAGA | linearized |
| | | CTTTCCTGCGCGTTTTGG (SEQ ID | pK18mobsacB. |
| | | NO: 35) | Regions in italics |
| | Amplify | Saro3873-8 HiFi Del F | or underlined |
| | downstream | CCCAAAACGCGCAGGAAAGTCTCCTG | italics are |
| | of Saro_3878 | GTTATCCCAAGATTCTCCAGCAATAAG | complementary to |
| | | GCTC (SEQ ID NO: 36) | each other. |
| | | Saro_3877-8 Ampl R pk18 HiFi | |
| | | GTTTCTGCGGACTGGCTTTCTAGA | |
| | | TGTTCCGTAAAGGTTCCAGTAGCCT | |
| | | AGTCCG (SEQ ID NO: 37) | |
| Inserting | Linearize | Saro_2812-3 Del R | Regions in bold |
| Saro_3877-8 | pK18msB- | CTGACCAGACAGGAGTAGTACCC | or italics are |
| into the | ΔSaro2812/3 | ATG (SEQ ID NO: 38) | complementary to |
| Saro_2812-3 | | Saro_2812-3 Del F | each other. |
| genomic locus | | CAtAGGCCCCTCTCCTTCAGCTTG | Underlined |
| | | (SEQ ID NO: 39) | regions are start |
| | Amplify | Saro3877-8-LigAB R | or stop codons. |
| | Saro_3877-8 | CGCATGGGTACTACTCCTGTCTGG | The lowercase |
| | | TCAGCTTATTGCTGGAGAATCTTGG | letter is non- |
| | | GATAACGCTG (SEQ ID NO: 40) | complementary to |
| | | Saro3877-8-LigAB F | its initial target |
| | | CCAAGCTGAAGGAGAGGGGCCTATGA | sequence. |
| | | CCATGAACGATCTCCCTAACC (SEQ | |
| | | ID NO: 41) | |
| Inserting | Linearize | Saro_2812-3 Del F2 | Regions in bold |
| Saro_3873 | pK18msB- | CACAGGCCCCTCTCCTTCAGCTTG | or italics are |
| and | 3877- | (SEQ ID NO: 42) | complementary to |
| Saro_3877-8 | 8ΔligAB | Saro3877-8_HiFi 3873 F | each other. |
| into the | | GGCAAATGGAAGGGAATTGAGAATGA | Underlined |
| Saro_2812-3 | | CCATGAACGATCTCCCTAACC (SEQ | regions are start |
| genomic locus | | ID NO: 43) | or stop codons. |
| | Amplify | Saro3873 pVP HiFi Ampl F | |
| | Saro_3873 | CAAGCTGAAGGAGAGGGGCCTGT | |
| | | GAAGCGCATGGTCGTGGGGATTAC | |
| | | (SEQ ID NO: 44) | |
| | | Saro3873 expr Ampl R | |
| | | CATTCTCAATTCCCTTCCATTTGCCCA | |
| | | GCACCCGGAAC (SEQ ID NO: 45) | |
| Deletion of | Upstream of | Saro3857_pK18_Amp_F | Bold and bold |
| Saro_3857 | xylE | GTTTCTGCGGACTGGCTTTCTACG | underlined |
| (xylE) | | TGTTCCGTTCATTACTTCACCCAGC | sequences are |
| | | AGGGC (SEQ ID NO: 46) | complementary to |
| | | Saro3857_pK18_Hifi_del_R | the end regions of |
| | | GGTCTCAAAGGCTGAACggaaaggGCAA | linearized |
| | | GGCGATCTTCTACTACGAAAAGG (SEQ | pK18mobsacB. |
| | | ID NO: 47) | Regions in italics |
| | Downstream | Saro3857_pK18_Hifi_del_F | or underlined |
| | of xylE | CCTTTTCGTAGTAGAAGATCGCCTTGC | italics are |
| | | cctttccgtTCAGCCTTTGAGACC (SEQ ID | complementary to |
| | | NO: 48) | each other. |
| | | Saro3857_pK18_Amp_R | |
| | | CGATTCATTAATGCAGCTGGCACG | |
| | | ACAGCGAAGGTCTCATCTGATCGAA | |
| | | GAGCG (SEQ ID NO: 49) | |
| Deletion of | Upstream | pK18_amp_delSaro3829_F | Bold and bold |
| Saro_3828- | Fragment | GTTTCTGCGGACTGGCTTTCTACG | underlined |
| 3829 | | TGTTCTCGCCGCAAGCTAGGACCGC | sequences are |
| | | (SEQ ID NO: 50) | complementary to |
| | | Del_Saro3828-3829_R | the end regions of |
| | | TCGTTGCTTGCCACATCGAAGATCGAC | linearized |
| | | GGCCACAGGACTAAGCGTTGC (SEQ | pK18mobsacB. |
| | | ID NO: 51) | Regions in italics |
| | Downstream | Del_Saro3828-3829_F | or underlined |
| | Fragment | GCAACGCTTAGTCCTGTGGCCGTCGAT | italics are |
| | | CTTCGATGTGGCAAGCAACGA (SEQ ID | complementary to |
| | | NO: 52) | each other. |
| | | pK18_amp_delSaro3829_R | |
| | | CGATTCATTAATGCAGCTGGCACG | |
| | | ACAGCGTTATGTGGTGATTTCAGCG | |
| | | ATCGTCG (SEQ ID NO: 53) | |
| Insertion of | Linearizing | XylE_catA_ATW_F | Regions in bold |
| catA into | ΔxylE pK18 | CGGACTACTCCCGTTATGTGGTGA | and bold |
| ΔxylE | plasmid | TCAGCCTTTGAGACCATTCCTAAAG | underline are |
| | | AA (SEQ ID NO: 54) | complementary to |
| | | XylE_catA_ATW_R | each other. |
| | | CAAGGCGATCTTCTACTACGAAAA | Italicized region |
| | | GGCGCTGAACGACCGCTTCATGACG | is a ribosomal |
| | | G (SEQ ID NO: 55) | binding site from |
| | Insertion of | XylE_Saro3830_hifi_F | the native catA |
| | Saro3830 | TTTCGTAGTAGAAGATCGCCTTGC | operon. |
| | (NacatA) into | TCAGGCCTGGGCGCGGGTGCGTTCC | |
| | ΔxylE | (SEQ ID NO:56) | |
| | | XylE_Saro3830_hifi_R | |
| | | TCACCACATAACGGGAGTAGTCCGAT | |
| | | GCCTGCCACCTTCGCCAGTTCCGAT | |
| | | (SEQ ID NO: 57) | |
| | Insertion of | XylE_EcCatA_hifi_F | |
| | EcCatA into | TTTCGTAGTAGAAGATCGCCTTGT | |
| | ΔxylE | CATTCCTTGACGCGGGCGCGCTTGC | |
| | | CG (SEQ ID NO: 58) | |
| | | XylE_EcCatA_hifi_R | |
| | | TCACCACATAACGGGAGTAGTCCGAT | |
| | | GTCGAAGAACCCGTCGCAGCAGTCG | |
| | | (SEQ ID NO: 59) | |

E. coli DH5α cells were used in all plasmid preparations and either E. coli S17 or E. coli WM6026 were used as a conjugal donor for mobilization of DNA into N. aromaticivorans. Procedures for conjugation and modifying the N. aromaticivorans genome via homologous recombination are found below. All E. coli strains were grown in Lysogeny Broth (LB) media containing 50 mg/L kanamycin or 0.3 mM diaminopimelic acid (DAP) when necessary. All N. aromaticivorans strains were grown in SMB minimal media (60) and supplemented with 10 mM glucose and an additional aromatic when specified. For genomic modifications of N. aromaticivorans the media was either supplemented with 50 mg/L of kanamycin or with 10% sucrose (100 g/L).

N. aromaticivorans Growth Experiments

Starter cultures of the N. aromaticivorans strains were grown aerobically (˜18 h) in 5 mL of SMB media supplemented with 10 mM glucose, in 18×150 mm culture tubes at 30° C. The cells were then diluted by 1:1 and regrown to mid-exponential growth phase. The SMB vanillic acid and SMB PCA solutions were prepared fresh by dissolving either 34 mg of vanillic acid or 30 mg of PCA into 100 mL of SMB, which was then passed through a sterile 0.22 m filter. A 1:10 dilution was performed by adding 1.2 mL of starter culture into a 125 mL Erlenmeyer flask equipped with a side arm that contained 12 mL of SMB media with the specified carbon source. The cell density was measured at various time points using a Klett-Summerson photoelectric colorimeter with a red filter (31). Aliquots of culture samples (0.2 mL) were removed at indicated time points and filtered (31) prior to storage at 4° C. Liquid chromatography mass spectroscopy (LCMS) analysis was performed on the day of the last time point.

### Protein Expression and Purification

Genes Saro_3877-78 and Saro_3830 were amplified via PCR from the N. aromaticivorans genome and independently cloned into the pVP302K plasmid, which contains an 8× His tag (32). EcaroY/D was PCR amplified from the ΔligAB1:EcDec_pK18mobsacB plasmid, and eccatA was PCR amplified out of a pUC57 plasmid synthesized by Genscript. Both genes were independently cloned into the pVP32K plasmid. The list of primers used to generate these protein expression plasmids and corresponding plasmids are shown in Table 5. Purified plasmid was then transformed into E. coli B834 containing the pRARE2 plasmid (32). Identical methods for heterologous protein expression and purification were performed and detailed below.

TABLE 5
Primers used for protein expression. The regions in bold are complementary
to the linearized regions of the pVP302K plasmid. All primers are listed in
the 5′ to 3′ orientation.

| Primers | Sequence | Plasmid |
|---|---|---|
| EcdB_pVP_HiFi_F | GTATTTTCAGAGCGCGATC | EcdB_pVP302K |
| | GCAGGAATGAGGCTCATCG | |
| | TGGGCATGACG (SEQ ID | |
| | NO: 60) | |
| EcdB_pVP_HiFi_R | CTAACTTTGTTATTTTCGG | EcdB_pVP302K |
| | CTTTCTGTCAGATGCCGTCG | |
| | TTGTTCTCCTGG (SEQ ID | |
| | NO: 61) | |
| EcAroY_EcdD_pVP_HiFi_F | GTATTTTCAGAGCGCGATC | EcAroY_D_pVP302K |
| | GCAGGAATGCAGAACCCCA | |
| | TCAACGACCTC (SEQ ID | |
| | NO: 62) | |
| EcAroY_EcdD_pVP_HiFi_R | CTAACTTTGTTATTTTCGG | EcAroY_D_pVP302K |
| | CTTTCTGGCTCACAGGAGC | |
| | GGAGGAATCGTG (SEQ ID | |
| | NO: 63) | |
| Saro3830_pVP_HIFI_F2 | GTATTTTCAGAGCGCGATC | NaCatA_pVP302K |
| | GCAATGCCTGCCACCTTCGC | |
| | CAG (SEQ ID NO: 64) | |
| Saro3830_pVP_HIFI_R2 | CTAACTTTGTTATTTTCGG | NaCatA_pVP302K |
| | CTTTCTGTCAGGCCTGGGCG | |
| | CGGGTG (SEQ ID NO: 65) | |
| Saro_3877_8_pVP_F | GTATTTTCAGAGCGCGATC | NadCD_pVP302K |
| | GCAATGACCATGAACGATCT | |
| | CCCTAACC (SEQ ID NO: 66) | |
| Saro_3877_8_pVP_R | CTAACTTTGTTATTTTCGG | NadCD_pVP302K |
| | CTTTCTGTTATTGCTGGAGA | |
| | ATCTTGGGATAAC (SEQ ID | |
| | NO: 67) | |
| EcCatA_PVP_HIFI_F | GTATTTTCAGAGCGCGATC | EcCatA_pVP302K |
| | GCAGGAATGTCGAAGAACC | |
| | CGTCGCAGCA (SEQ ID | |
| | NO: 68) | |
| EcCatA_pVP_HIFI_R | CTAACTTTGTTATTTTCGG | EcCatA_pVP302K |
| | CTTTCTGTTCCTTGACGCGG | |
| | GCGCGCTTG (SEQ ID NO: 69) | |

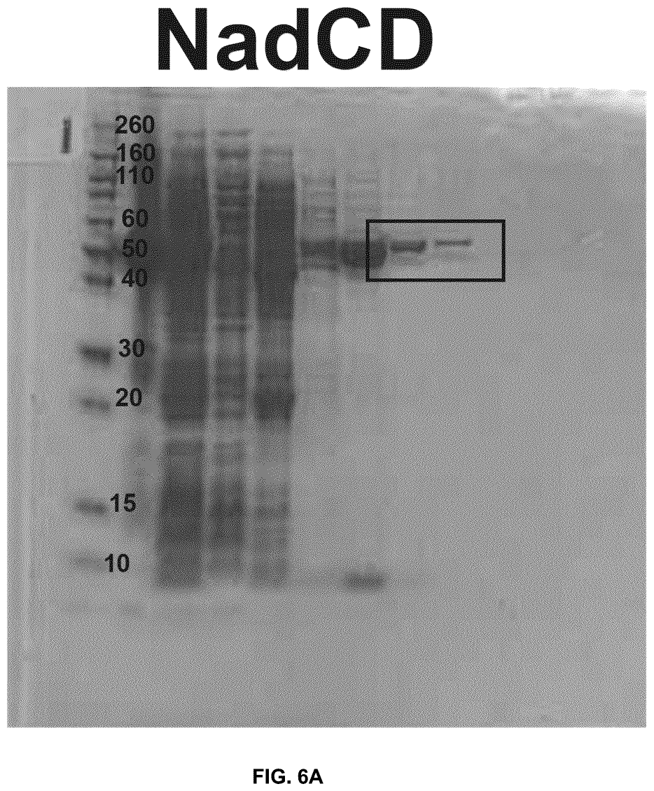

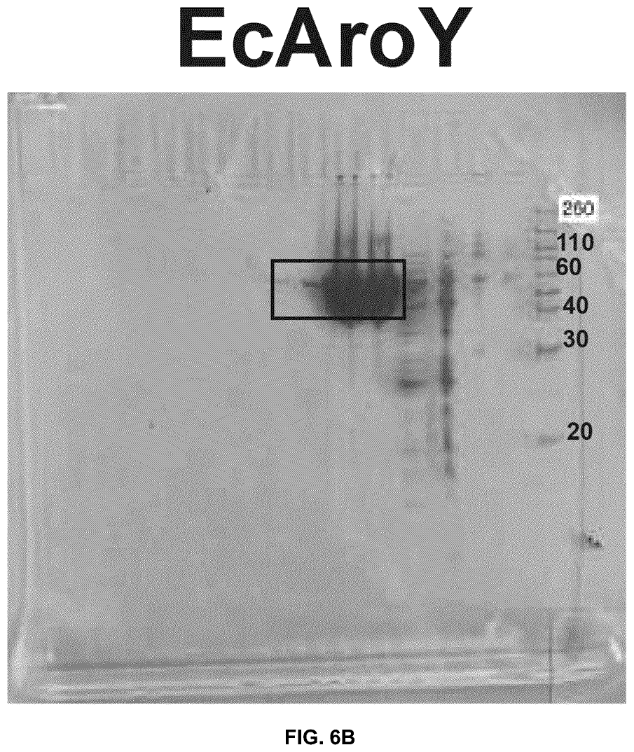

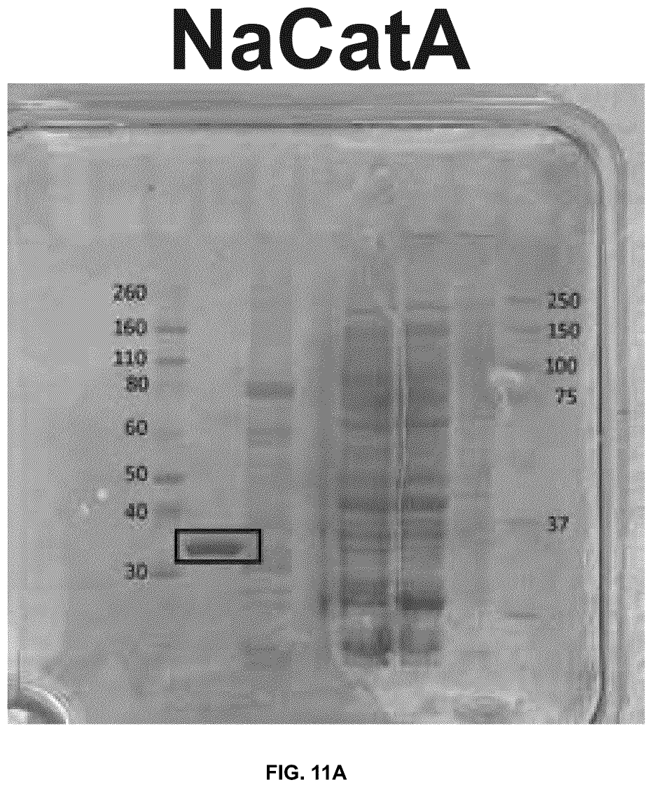

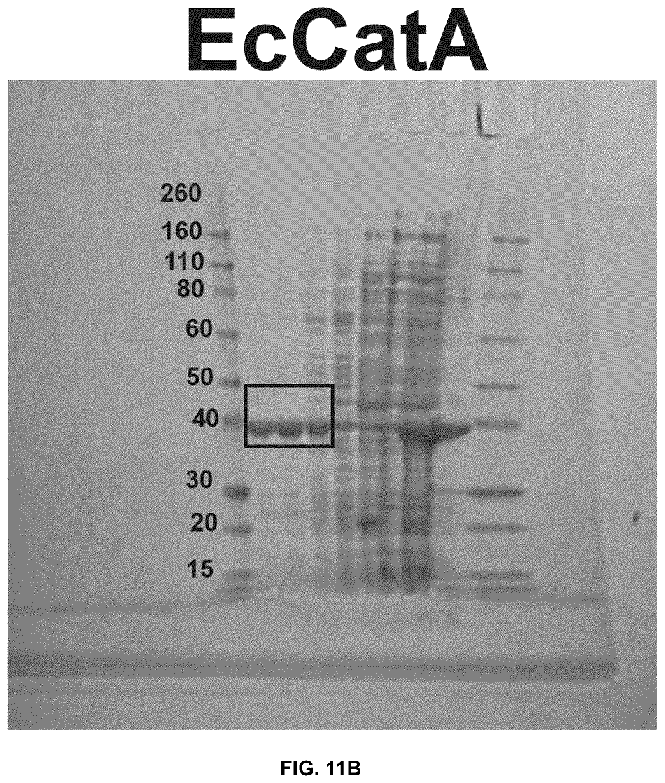

For protein expression, a single colony was used to inoculate a 20 mL starter culture of cells grown in LB media containing kanamycin (50 mg/L) and chloramphenicol (20 mg/L) in a 125 mL Erlenmeyer flask that was shaken at 200 rpm overnight (˜18 h) at 37° C. Next, the entire 20 mL starter culture was used to inoculate a 2 L Erlenmeyer flask containing 500 mL of Terrific Broth (TB) (61) media containing kanamycin (50 mg/L) and chloramphenicol (20 mg/L). The 500-mL culture was shaken at 200 rpm at 37° C. for 4 to 5 h until reaching an optical density (GD) OD₆₀₀ of ˜0.7. Once the cells reached an OD₆₀₀ of 0.7, protein expression was induced with the addition of Isopropyl β-D-1-thiogalactopyranoside (IPTG, 0.3 mM final concentration). For expression of the 1,2-catechol dioxygenase proteins, the media also included Fe(II)SO₄ at a final concentration of 0.160 mM at the time of induction. Induction was allowed to proceed overnight (˜18 h) at 20° C. for both the PCA decarboxylase and the 1,2 catechol dioxygenase cultures. After induction, cells were harvested by centrifugation and suspended in the resuspension buffer which contains 50 mM HEPES (2-[14-(2-hydroxyethyl)piperazin-1-yl]ethane-1-sulfonic acid), 150 mM NaCl and 0.1% Triton X-100 at pH 7.5. The cells were then lysed by sonication and clarified by centrifugation at 20,000 rpm for 30 min. The soluble fraction was applied directly to a Ni-NTA column and washed with 50 mM HEPES, 150 mM NaCl and 30 mM Imidazole at pH 7.5. The proteins were eluted by applying a high imidazole elution buffer (50 mM HEPES, 150 mM NaCl and 300 mM Imidazole at pH 7.5). Fractions were collected and protein purity was analyzed by sodium dodecyl sulfate polyacrylamide gel electrophoresis (FIGS. 6A, 6B, 11A, and 11B). The protein concentration was determined by the Bradford protein assay measuring the absorbance at 595 nm (Fisher Scientific).

Generation of a prFMN-Containing Crude Cell Lysate

A source of prFMN was generated as previously described (62) with slight modification. The gene for the EcdB prenyl transferase was PCR amplified from the dLigAB1:EcDec pK18 plasmid, and cloned into pVP302K. The resulting expression vector was then transformed into B834 E. coli containing the pRARE2 plasmid. A single colony of the EcdB BP86 E. coli was used to inoculate 10 mL of LB media containing kanamycin (50 mg/L) and chloramphenicol (20 mg/L). The culture was incubated for ˜18 h at 37° C. with shaking (200 rpm). Next, the 10-mL culture was used to inoculate 1 L of TB kanamycin and chloramphenicol media and incubated with shaking for 5 to 6 h until an OD of 0.7 was reached. The culture was then transferred to 1 L screw top bottle with a magnetic stir bar for anaerobic growth and was amended to include a final concentration of 1% dimethylsulfoxide (DMSO), 1 mM prenol, 0.1 mM riboflavin and 0.4 mM IPTG as previously described (62). After incubation overnight (˜18 h) the cells were then lysed via sonication and clarified by centrifugation. The resulting crude cell lysate (prFMN lysate) was used as a source of prFMN for the PCA decarboxylase activity assays.

### PCA Decarboxylase In Vitro Activity Assays

All PCA decarboxylase activity assays were performed in triplicate in the reaction buffer (50 mM HEPES, 150 mM NaCl pH 7.5) using purified enzymes. A stock solution of 25 mM PCA was generated by dissolving 39 mg into 10 mL of reaction buffer. To test the dependence of NadCD activity on prFMN, reactions were initiated by adding 0.1 μM (final concentration) NadCD to a 2 mL (final volume) reaction mixture that contained 1 mM PCA. For reactions that included the prFMN crude cell lysate, the assay mixture also contained 1 mL of prFMN lysate (50% lysate) and 1 mM PCA at a final volume of 2 mL. The enzyme assay was quenched by the addition of 40 μL of 1M HCl to a 0.2 mL aliquot of the reaction mixture at T=0 and T=18 h. For temporal analysis of PCA decarboxylase activity by NadCD or EcAroY, those reaction were initiated by the addition of enzyme to final concentration of 0.1 μM and added to the reaction mixture that included the prFMN crude cell lysate detailed above. Aliquots of 0.2 mL were removed and the reaction terminated as above at various time points. A control reaction of the prFMN lysate reaction mixture without the addition of enzyme was also performed. All reaction products were filtered through 0.22 μm nylon syringe tip filter (Fisher Scientific) and analyzed by LCMS to test for PCA to catechol conversion.

### 1,2 Catechol Dioxygenase In Vitro Activity Assays

Catechol dioxygenase activity was tested in triplicate with either NaCatA or EcCatA in a reaction buffer of 50 mM HEPES, 150 mM NaCl pH 7.5. A stock solution of 0.1 M catechol was freshly prepared by dissolving 22 mg into 2 mL of the reaction buffer. A series of dilutions were performed on the stock solution to obtain a 1 mM catechol working solution. The reactions were performed in a 96-well plate in a total volume of 200 μL with orbital shaking at 28° C. using a Tecan infinite M1000 Pro to ensure O₂ dissolution. The reaction mixture contained 0.5 μM of purified enzyme in reaction buffer and the assay was initiated by the addition of catechol. The formation of ccMA was monitored at 260 nm (50) and the resultant data were best fit to a linear equation ([ccMA]=kt+[ccMA]₀) to yield zeroth order rate constants (k) for each enzyme.

N. aromaticivorans Extracellular Metabolite Analysis by HPLC-MS Extracellular metabolite analysis was carried out on a Shimadzu triple-quadrupole liquid chromatography-mass spectrometer (LC-MS, Nextera XR HPLC-8045 MS/MS). The mobile phase used a binary gradient with solvent A (0.2% formic acid in water) and solvent B (acetonitrile) using the protocols listed as Method 1 in Table 6 and Method 2 in Table 7.

TABLE 6
Liquid chromatography (LC) program for
elution of phenolics using Method 1.

| Time (min) | % B conc |
|---|---|
| 0.05 | 0 |
| 1.9 | 0 |
| 3 | 0 |
| 3.25 | 2 |
| 3.5 | 4 |
| 3.75 | 6 |
| 4 | 8 |
| 4.25 | 10 |
| 4.5 | 12 |
| 4.75 | 14 |
| 5 | 16 |
| 6 | 20 |
| 7.5 | 50 |
| 8 | 95 |
| 9 | 95 |
| 9.25 | 5 |
| 10 | 0 |
| 11 | End of Run |

TABLE 7
LC program for elution of phenolics in Method 2.

| Time (min) | % B conc |
|---|---|
| 0.05 | 5 |
| 3.5 | 8 |
| 4 | 11 |
| 5.5 | 12 |
| 6.5 | 14 |
| 7 | 30 |
| 7.5 | 50 |
| 8 | 75 |
| 8.5 | 95 |
| 9.25 | 95 |
| 9.5 | 5 |
| 10 | 5 |
| 10.5 | 1 |
| 11 | End of run |

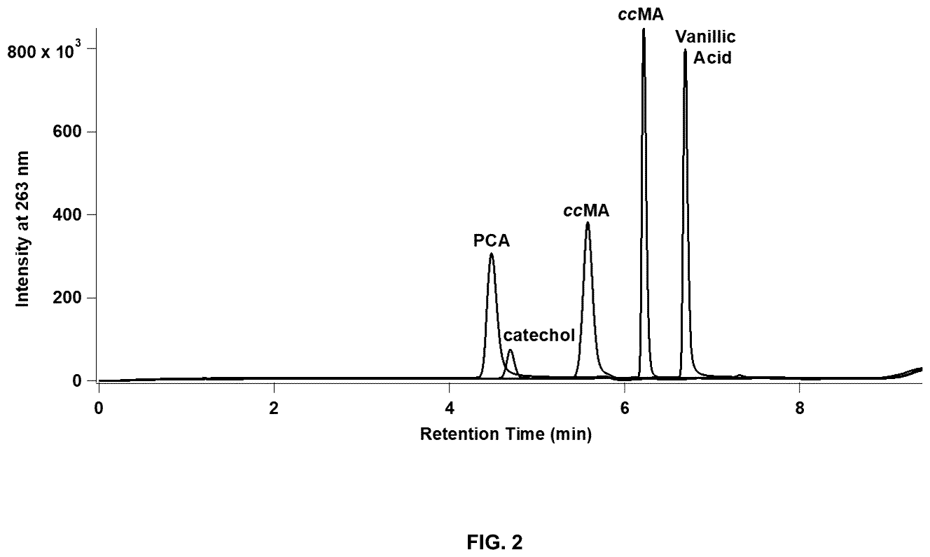

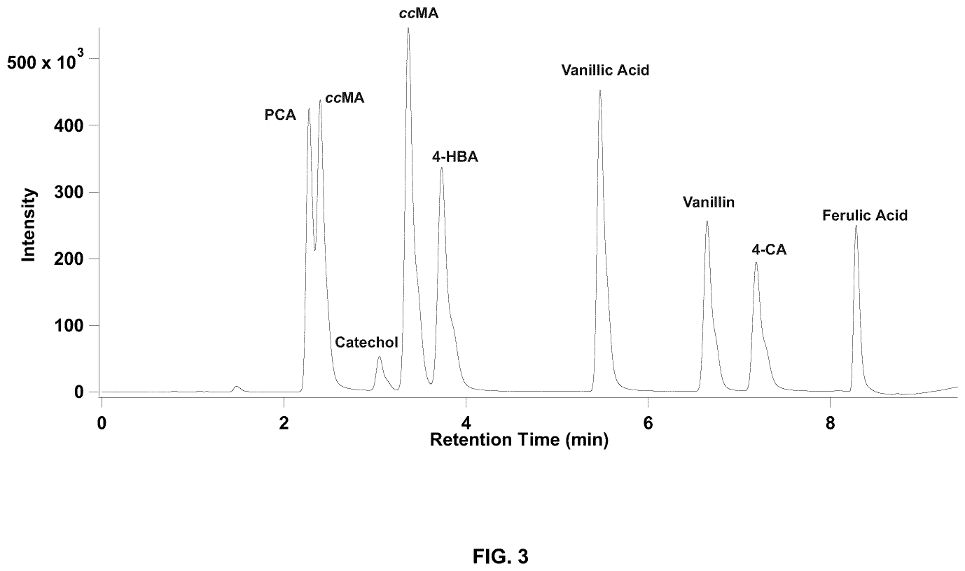

The stationary phase used was a Kinetex C18 column (Kinetex 2.6 μm pore size, 100 Å 150 length×2.1 mm ID, P/N: 00F-4462-AN). Quantification of the metabolites was performed by preparing standard solutions of compounds (Sigma-Aldrich). A series of dilutions were performed to obtain a set of 5 concentrations for each compound that was within the range of the predicted amount of analyte (FIGS. 2 and 3). The compounds were analyzed by both UV/vis absorption and multiple-reaction-monitoring (MRM) (Tables 8 and 9). Each of the compounds were quantified using the maximum absorbance wavelength in the UV/visible absorption spectrum and a standard curve was applied using the area under the curve at that maximum absorbance (Tables 8 and 9).

TABLE 8
Multiple Reaction Monitoring (MRM) transitions and
maximum UV/vis absorption data used to quantify phenolics
in Method 1 (see Materials and Methods).

| | Retention | Maximum UV/vis | |
|---|---|---|---|
| Compound | Time | absorption | MRM m/z |
| Vanillic acid | 6.692 | 263 nm | 167.00 > 152.10 |
| PCA | 4.473 | 263 nm | 153.00 > 109.10 |
| Catechol | 4.701 | 275 nm | 109.20 > 91.10 |
| ccMA 1 | 5.570 | 263 nm | 141.15 > 97.10 |
| ccMA 2 | 6.217 | 263 nm | 141.15 > 97.10 |

TABLE 9
Multiple Reaction Monitoring (MRM) transitions and
maximum UV/vis absorption data used to quantify extracellular
aromatics in Qsub poplar APL Method 2.

| | Retention | Maximum UV/vis | |
|---|---|---|---|
| Compound | Time | absorption | MRM m/z |
| 4-CA | 7.186 | 309 nm | 163.00 > 119.10 |
| 4-HBA | 3.732 | 254 nm | 136.90 > 93.10 |
| Catechol | 3.049 | 275 nm | 109.20 > 91.10 |
| ccMA 1 | 2.400 | 263 nm | 141.15 > 97.10 |
| ccMA 2 | 3.367 | 263 nm | 141.15 > 97.10 |
| Ferulic Acid | 8.283 | 322 nm | 193.00 > 134.10 |
| PCA | 2.277 | 263 nm | 153.00 > 109.10 |
| Vanillic acid | 5.473 | 263 nm | 167.00 > 152.10 |
| Vanillin | 6.647 | 309 nm | 151.00 > 136.00 |

The percent yields for ccMA were obtained by using the equation below, and the initial aromatic concentrations refers to the aromatic carbon that was used in the growth experiments.

$$percent yield = \frac{{[ ccMA ]}_{final}}{{[ aromatic ]}_{initial}} \times 100$$

### Alkaline Pre-Treatment Liquor Preparation

The line 15.1 of QsuB poplar was obtained and treated to create APL as previously described (51, 52). The total phenolics in APL were calculated as the sum of the free phenolics and the glycosylated phenolics released after acid treatment of the APL (52). Growth experiments in the presence of APL were performed after adjusting to pH to 7.0 with hydrochloric acid and supplementing with glucose (1 g/L) and ammonium sulfate (1 g/L) (52). The percent yield of ccMA production was calculated as indicated above.

### Construction of Plasmids for Generating Deletions of Saro_3877-8 and Saro_3873-8

Regions of N. aromaticivorans genomic DNA containing ˜1000 base pairs (bp) upstream and downstream of the genes to be deleted were amplified via PCR using the primers listed in Table 4. Plasmid pK18mobsacB was linearized via PCR as previously described (60). The upstream and downstream flanking regions for each gene were combined with linearized pK18mobsacB using the NEBuilder HiFi Assembly system (New England Biolabs, Ipswich, MA) to produce a plasmid in which the upstream and downstream DNA sequences are adjacent, with no intervening coding region. For the strain in which Saro_3873-8 was deleted, the deleted region begins two bp before the start codon of Saro_3873. For the strain in which Saro_3877-8 was deleted, the deleted region begins one bp before the start codon of Saro_3877. In both strains, the deletion extends to the same point near the end of Saro_3878. The final 26 bp of Saro_3878 remain in the N. aromaticivorans genome.

Construction of a Plasmid to Insert Saro_3877-8 into the Saro_2812-3 Locus of the N. aromaticivorans Genome

Plasmid pK18mobsacB/ΔSaro2812/3 (32) which contains ˜1,000 bp genomic regions upstream and downstream of Saro_2812-3, was linearized via PCR using primers shown in Table 4. Concurrently, Saro_3877-8 was amplified from N. aromaticivorans genomic DNA via PCR using primers containing upstream regions complementary to linearized pK18mobsacB/ΔSaro2812/3 (Table 4). The linearized pK18mobsacB/ΔSaro2812/3 and the Saro_3877-8 fragment were combined using the NEBuilder HiFi Assembly system (New England Biolabs, Ipswich, MA) to produce plasmid pK18mobsacB-3877-8ΔligAB, in which Saro_3877-8 was placed between the DNA regions that naturally flank Saro_2812-3. The Saro_3877 start codon is in the natural position for the Saro_2813 start codon, and the Saro_3878 stop codon is in the natural position of the Saro_2812 stop codon, with an additional C following the Saro_3878 stop codon that does not naturally follow the Saro_2812 stop codon. The Saro_2814 stop codon (UGA), which naturally overlaps with the Saro_2813 start codon, remains intact and now overlaps with the Saro_3877 start codon.

Construction of a Plasmid to Insert Saro_3873 and Saro_3877-8 into the Saro_2812-3 Locus of the N. aromaticivorans Genome

Plasmid pK18mobsacB-3877-8ΔligAB was linearized via PCR using primers in Table 4. Concurrently, Saro_3873 was amplified from N. aromaticivorans genomic DNA via PCR using primers containing upstream regions complementary to linearized pK18mobsacB-3877-8ΔligAB (Table 4). The linearized pK18mobsacB-3877-8ΔligAB and the Saro_3873 fragments were combined using the NEBuilder HiFi Assembly system (New England Biolabs, Ipswich, MA) to produce plasmid pK18mobsacB-3873/7-8ΔligAB, in which Saro_3873 and Saro_3877-8 formed an artificial operon between the DNA regions that naturally flank Saro_2812-3. The Saro_3873 start codon is in the natural position for the Saro_2813 start codon. The 2 bp intergenic region normally between Saro_3873 and Saro_3874 follows the Saro_3873 stop codon, and is now followed by the Saro_3877 start codon.

Plasmids for Inserting Genes Encoding the EcdB, EcAroY, and EcdD Proteins from Enterobacter cloacae into the Saro_2812-3 Locus of the N. aromaticivorans Genome

DNA fragments containing the genes coding for EcdB (NCBI Accession: ADF63617), EcAroY (NCBI Accession: ADF61496), and EcdD (NCBI Accession: ADF63615) from Enterobacter cloacae were ordered as gBlocks from Integrated DNA Technologies (Coralville, IA). Genes were constructed to have codon usage frequencies similar to those of other genes in N. aromaticivorans (calculated from several genes in the genome), but without making the GC content of the fragments too high for the gBlock synthesis process. The DNA sequence of the operon was split between two gBlocks (EcdB-aroY-D-NaligAB_up and EcdB-aroY-D-NaligAB_down); each gBlock contained a sequence at one end that matches one of the ends of linearized pK18mobsacB-ΔSaro2812/3; the other ends of the gBlocks match each other. The two gBlocks were combined with linearized pK18mobsacB-ΔSaro2812/3 using the NEBuilder HiFi Assembly system. The ecdB start codon is in the natural position for the Saro_2813 start codon, and the ecdD stop codon is in the natural position of the Saro_2812 stop codon.

Construction of Plasmids ΔxylE_pK18mobsacB and ΔcatBC_pK8mobsacB

Primers for deletion of catBC and xylE (Table 4) were designed to amplify two ˜1000 bp regions both downstream and upstream of the desired gene deletion in the genome of N. aromaticivorans. For each plasmid, these two amplified regions were combined with linearized pK18mobsacB-MCS1 as described above using NEBuilder® Hifi DNA assembly Master mix (New England Biolabs). For ΔcatBC, the deletion begins 1 bp upstream of the start codon of saro_3828 with the final 133 bp of saro_3829 remaining in the genome. The deleted region of ΔxylE begins 9 bps upstream of the start codon of saro_3857 (xylE) with the final 62 bp of saro_3857 remaining in the genome.

Construction of Plasmids ΔxylE eccatA and ΔxylE nacatA pK18mobsacB

For insertion of catA into the xylE locus of N. aromaticivorans, the dxylE_pK18mobsacB plasmid was first linearized using XylE_catA_ATW_F and XylE_catA_ATW_R. The XylE_CatA_ATW_F primer contained a ˜20 bp region that is located upstream of the native nacatA consistent with the ribosomonal binding site (RBS) for nacatA. Primers listed in Table 4 were used to amplify either eccatA or nacatA was out of the pVP302K vector with overhangs corresponding to the linearized ΔxylE_pK18mobsacB. Next, either fragment eccatA or nacatA was combined with the linearized ΔxylE_pK18mobsacB using Hifi DNA assembly reaction.

### Conjugation Procedure

Bacterial conjugations using either E. coli S17 or WM6026 donor cells with N. aromaticivorans recipients were performed essentially as previously described (26, 31) with slight modifications. Cultures of E. coli WM6026 cells were grown in LB kan and 0.3 mM DAP while N. aromaticivorans strains were grown in 10 mM glucose SMB media. Both strains were grown overnight at 30° C. in 5 mL of media in an 18×150 mm culture tube. The overnight cultures were then diluted 1:1 and grown to mid-log phase. The cell density of the donor and recipient cultures was then measured and the cultures were diluted such that the cell density of both the recipient and the donor were equal. Next, 2 mL of the donor and 1 mL of the recipient culture was subsequently washed and pelleted prior to mixing in a 2:1 donor to recipient cell ratio. The mixed cells were then suspended into 0.1 mL of LB DAP and incubated at 30° C. for 4 hr. After incubation, the cells were then pelleted by centrifugation and resuspended into 0.5 mL SMB supplemented with 10 mM glucose before incubating and shaking for an additional hour at 30° C. The cells were plated onto SMB Kan and transconjugant colonies formed within 3 to 4 days. Double crossover variants were selected as previously described (31) from SMB 10% sucrose plates. PCR amplified regions of the targeted genes were sequenced to confirm the mutation.

### Results

PCA Catabolism in N. aromaticivorans

The genome sequence of N. aromaticivorans predicts that it encodes enzymes which can convert both H- and G-biomass aromatics into PCA. Therefore, in order to test if we could engineer a strain that produces ccMA from H and G lignin aromatics, we first sought to develop further understanding of PCA catabolism in N. aromaticivorans. It was previously shown that the PCA 4,5 extradiol cleavage pathway is the major pathway in N. aromaticivorans when these cultures are supplied only an aromatic substrate (32). Additionally, it was shown that there are two 4,5 PCA dioxygenase homologues (LigAB1 and LigAB2) of the PCA extradiol cleavage pathway that can convert PCA to 4-carboxy-2-hydroxy-cis,cis-muconate-6-semialdehyde (CHMS) (FIG. 1) (32). Deletions of these genes predicted to encode enzymes in the PCA 4,5 extradiol cleavage pathway (FIG. 1) resulted in a N. aromaticivorans strain that accumulates extracellular PDC from H and G aromatics with yields greater than 70% when supplied an aromatic and glucose. These results agree with published data and demonstrate that this is a major pathway for the catabolism of H and G aromatics (31, 32). However, previous work also found less than stoichiometric yields of PDC when cells were grown with PCA as a carbon source (31). These results suggest that LigAB1 and LigAB2 play a significant role in PCA metabolism, but that the existence of another PCA-consuming pathway could be the cause of the decreased yield of PDC when this strain is grown with PCA. Based on these results, we hypothesized that blocking the N. aromaticivorans PCA extradiol cleavage pathway in the parent strain (12444, Table 3) would result in a strain that metabolizes PCA but at a rate slower than observed in cells that contain LigAB1 and LigAB2.

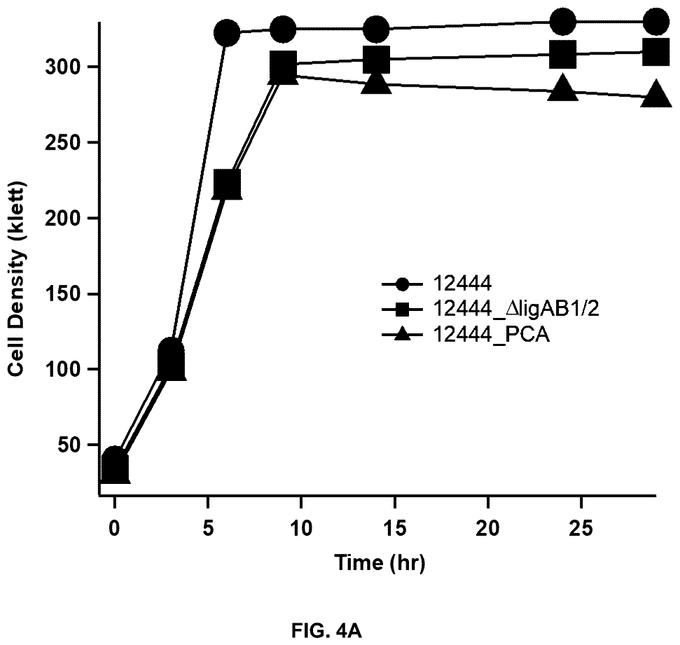

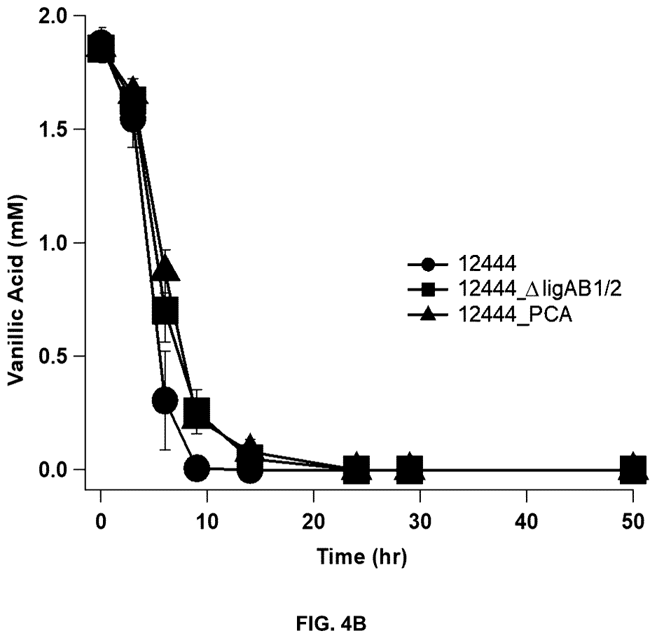

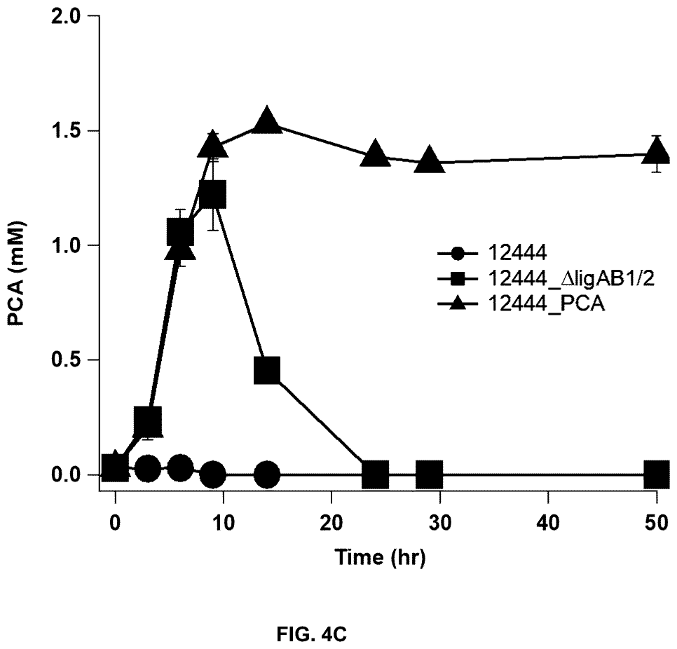

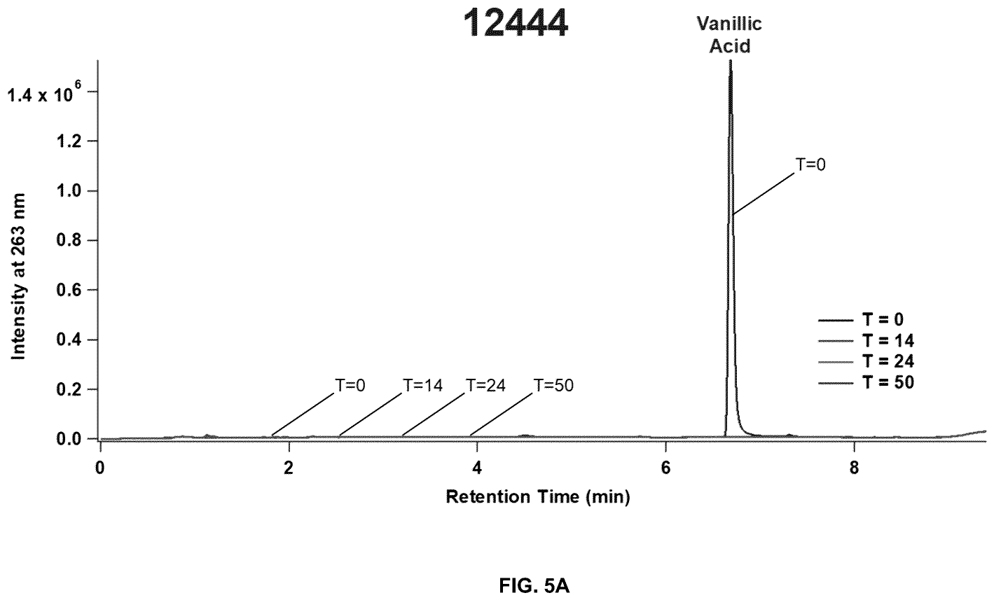

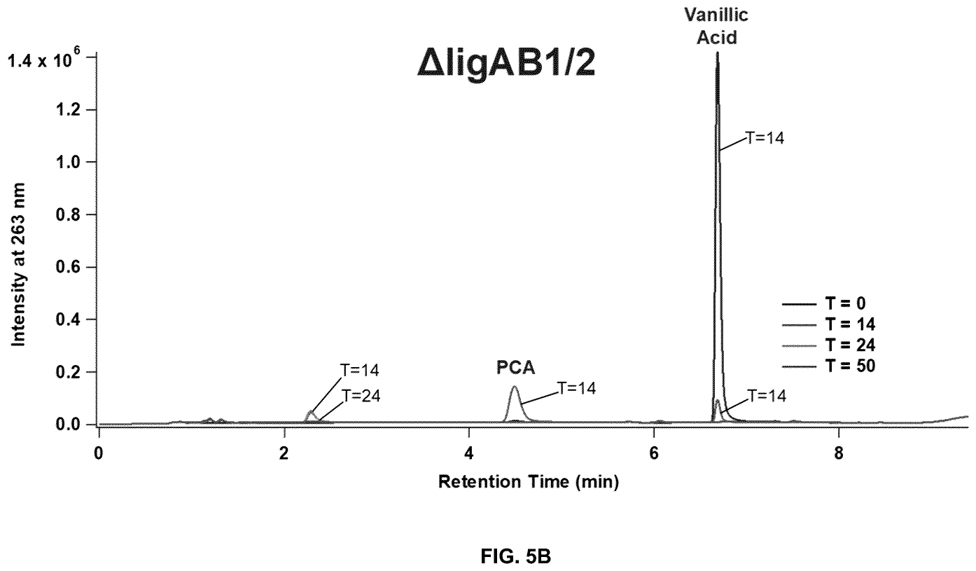

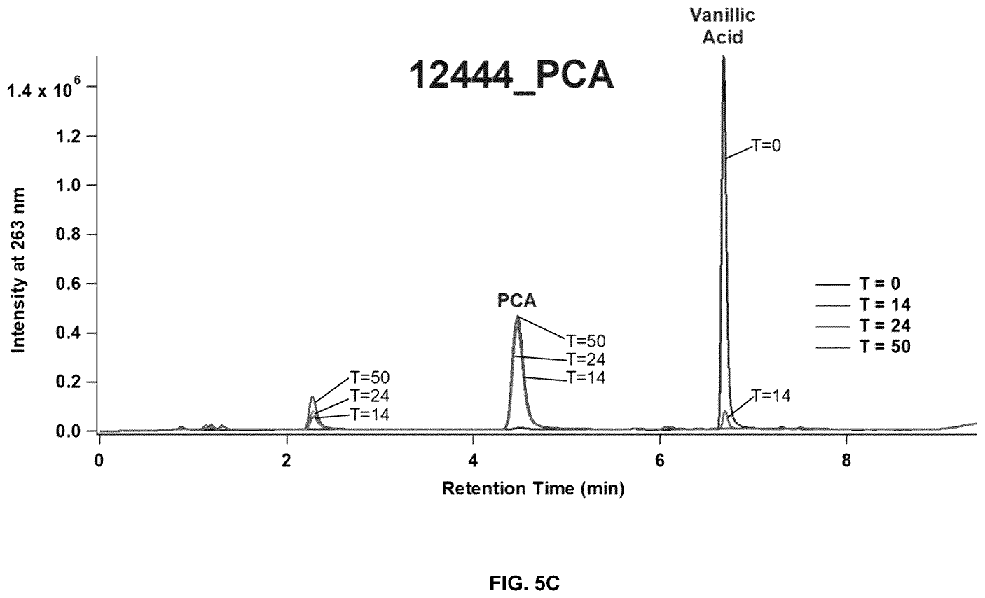

To test this hypothesis, we determined the effect of deletions in ligAB1 and ligAB2 on the ability to metabolize PCA in the presence of glucose as an auxiliary carbon source. This strain 12444_ΔligABΔligAB2 (32) (hereafter called 12444_ΔligAB1/2) and the parent strain 12444 (Table 3) were grown in media containing 2 mM vanillic acid, a G aromatic, and 10 mM glucose. Both strains grew to similar cell densities and fully eliminated the vanillic acid from the medium. As predicted, there was no extracellular accumulation of PCA in the 12444 parent strain (FIG. 4C, FIG. 5). However, the 12444_ΔligAB1/2 mutant transiently accumulated detectable levels of PCA in the media (FIG. 4C, FIG. 5B). The observation of the transient formation of PCA when the 12444_ΔligAB1/2 strain is grown with vanillic acid is consistent with the hypothesis that another route for metabolism of this predicted pathway intermediate is present (31, 32).

To test how PCA might be metabolized in the 12444_ΔligAB1/2 strain, we analyzed the genome of N. aromaticivorans for genes that encode homologues of enzymes known to catalyze PCA ring opening reactions in other organisms. This analysis failed to identify genes that encode proteins with >25% amino acid sequence identity to the Pseudomonas putida 3,4 dioxygenase (PcaHG) which catalyzes intradiol cleavage of PCA to form 3-carboxy-cis,cis-muconate or to a 2,3 PCA dioxygenase (PraA) that produces 5-carboxy-2-hydroxymuconate-6-semialdehyde (35-37). However, this analysis showed that N. aromaticivorans contained genes (Saro_3873, Saro_3877, Saro_3878; hereafter referred to as nadB, nadC, and nadD, respectively) that encoded proteins with at least some amino acid sequence identity to the known B, C and D gene products involved in PCA decarboxylation by Klebsiella pneumoniae, Enterobacter cloacae and other bacteria (Table 10) (5, 6, 38-40).

TABLE 10
Amino acid sequence identity of N. aromaticivorans gene products
with amino acid similarity to known PCA decarboxylases. Genes from either
Klebsiella pneumoniae or Enterobacter cloacae have been used
previously in bacterial engineering for ccMA production.

| | Saro_3873 | Saro_3877 | Saro_3878 |
|---|---|---|---|
| Amino Acid Sequence Identity | (NadB) | (NadC) | (NadD) |
| Enterobacter cloacae (EcdB) | 47% | | |
| Klebsiella pneumoniae (KpdB) | 44% | | |
| Enterobacter cloacae (EcdC) | | 45% | |
| Klebsiella pneumoniae (KpdC) | | 46% | |
| Enterobacter cloacae (EcAroY) | | 31% | |
| Klebsiella pneumoniae (KpAroY) | | 32% | |
| Enterobacter cloacae (EcdD) | | | 25% |
| Klebsiella pneumoniae (KpdD) | | | 27% |

We propose that a previously uncharacterized N. aromaticivorans PCA decarboxylase was responsible for metabolizing the PCA that transiently accumulated in the media of the 12444_ΔligAB1/2 strain. We performed a set of in vitro and in vivo experiments to test this proposal.

Characterization of N. aromaticivorans PCA Decarboxylase Enzyme

The amino acid sequence of the predicted N. aromaticivorans PCA decarboxylase enzyme (hereafter called NadBCD) predicts that it is most similar to a family of hydroxyarylic acid decarboxylases (38) that typically require 3 proteins, BCD, for activity. The B gene product encodes a predicted prenyltransferase that produces a prenylated flavin mononucleotide (prFMN) cofactor, while C catalyzes decarboxylation and D encodes a protein of unknown function (41, 42). Analysis of the most extensively studied decarboxylases in this family (Escherichia coli UbiD and Enterobacter cloacae EcAroY) have shown that the prFMN cofactor is required for decarboxylase activity (39, 43). Therefore, we tested if the N. aromaticivorans NadCD had PCA decarboxylase activity and whether it required a prFMN cofactor for catalysis (39).

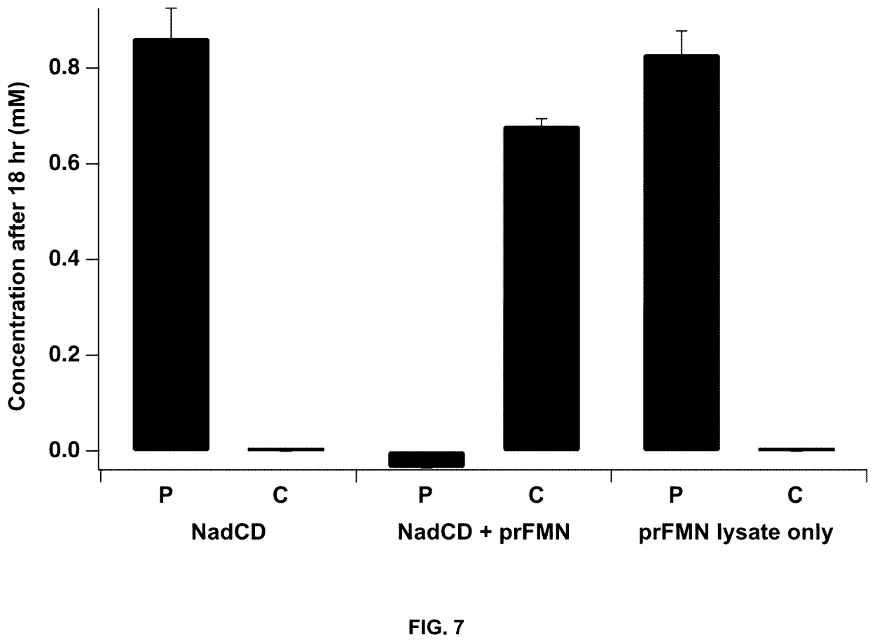

To test this hypothesis, Saro_3877 (nadC) and Saro_3878 (nadD) were amplified from the genome of N. aromaticivorans, cloned into expression vectors, and purified recombinant enzymes (FIG. 6A) were tested for PCA decarboxylase activity in vitro. Previously, researchers demonstrated that a source of prFMN can be produced by overexpression of the E. coli prenyltransferase when this bacterium is grown under anaerobic conditions in the presence of riboflavin (FMN precursor) and prenol (as a prenyl source) (44). In this study, we used a similar procedure, based on heterologous expression of the E. cloacae EcdB prenyltransferase in E. coli, to obtain crude cell extracts that contained prFMN. We then tested purified recombinant NadCD for PCA decarboxylase activity in the presence and absence of the crude cell extracts containing the predicted prFMN cofactor. As expected, we failed to observe detectable loss of PCA or production of catechol when purified NadCD was supplied 1 mM PCA with no source of prFMN (FIG. 7). However, when the same purified NadCD was mixed with PCA and with the crude E. coli lysate that contained prFMN, the PCA was converted into catechol (FIG. 7). We propose that the lack of stoichiometric conversion of PCA to catechol in these longer incubation assays reflects catechol degradation via its well-known abiotic oxidation reaction (21). A control reaction in which PCA was mixed with the prFMN-containing E. coli lysate and no purified recombinant NadCD failed to detect any loss of PCA or production of catechol over the same incubation period (FIG. 7). From these results, we conclude that NadCD has PCA decarboxylase activity and that, like most previously studied hydroxyarylic acid decarboxylases, requires prFMN for catalysis.

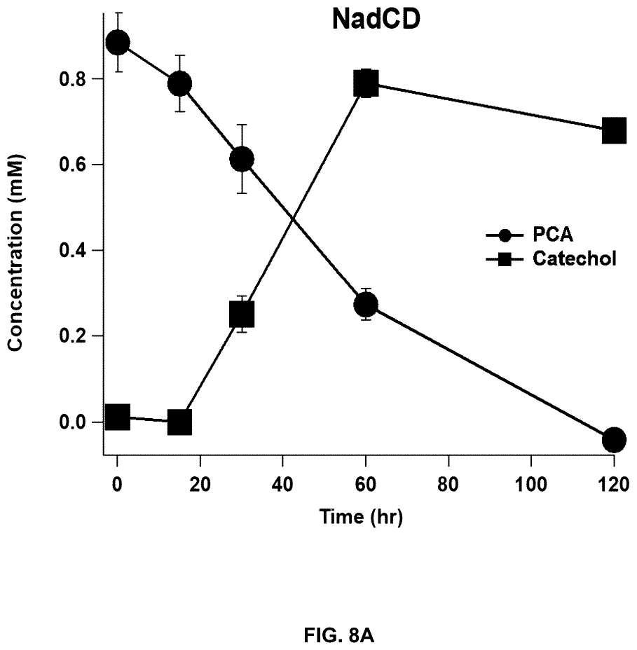

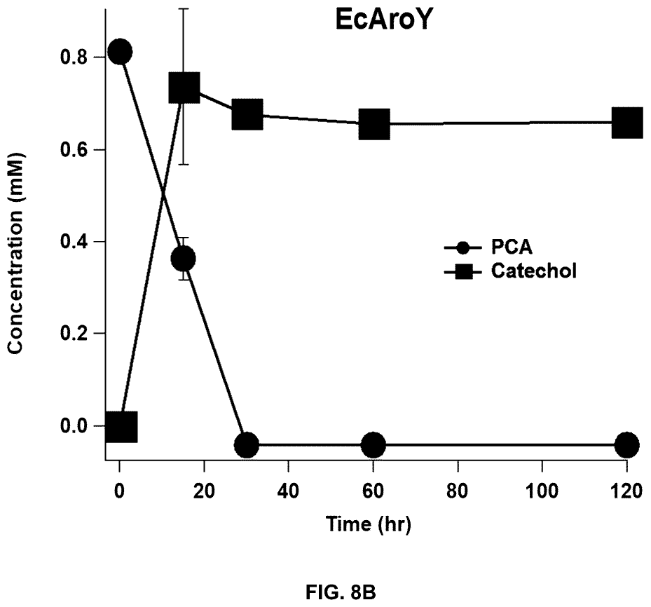

We also sought to compare the activity of NadCD to EcAroY, which is an extensively studied PCA decarboxylase from E. cloacae (39, 45). Temporal analysis of PCA decarboxylation by recombinant NadCD and EcAroY indicated that, under identical reaction conditions, EcAroY produced stoichiometric catechol from PCA within 15 min whereas 1 hour was needed for NadCD to produce stoichiometric levels of catechol (FIG. 8A). These results suggest that the recombinant N. aromaticivorans protein performs PCA decarboxylation slower than EcAroY in vitro. Overall, these results confirm the predicted function of N. aromaticivorans NadCD as a PCA decarboxylase that converts PCA to catechol. It also predicts that the NadCD decarboxylase activity was responsible for the observed PCA metabolism in the 12444_ΔligAB1/2 strain (FIG. 4C) and that a derivative of this mutant which also lacks the nadBCD genes should exhibit a defect in PCA metabolism.

Loss of Both nadBCD and ligAB1/2 is Sufficient to Accumulate Extracellular PCA

To test this hypothesis, we generated a strain which lacks both ligAB1/2 and the Saro_3873-8 gene cluster, which contains nadBCD (12444_PCA; Table 3). We grew the parent strain 12444, the 12444_ΔligAB1/2 and 12444_PCA mutants in media containing 2 mM vanillic acid and 10 mM glucose as an additional carbon source. We found all strains fully consumed vanillic acid but that strain 12444_PCA reproducibly grew to a lower cell density than 12444 strain or the 12444_ΔligAB1/2 mutant (FIG. 4A). We hypothesized that the lower cell density was due to failure of 12444_PCA to assimilate the products of vanillic acid metabolism. Analysis of the extracellular metabolites of 12444_PCA revealed that the vanillic acid was consumed at the end of the incubation and that the media contained 1.4±0.1 mM of PCA, representing an almost complete (78±5%) recovery of extracellular PCA from vanillic acid. We propose that the less than stoichiometric recovery of PCA in the media at the end of the incubation is due to the previously reported abiotic oxidation of PCA (46-48). From these results we concluded that NadBCD contributes to PCA metabolism of 12444 N. aromaticivorans. We further propose that the combined deletion of the nadBCD and ligAB1/2 genes generates a N. aromaticivorans strain (12444_PCA) that excretes PCA because it has a defect in assimilation of this intermediate in aromatic metabolism.

### Converting PCA to Catechol

Extracellular accumulation of PCA by strain 12444_PCA predicts that there are no other major pathways for PCA metabolism in N. aromaticivorans. This finding enabled us to use 12444_PCA as a platform strain to test if we could divert the PCA derived from H- and G-family aromatics towards ccMA production. Since bacterial production of ccMA often proceeds through the intradiol aromatic ring cleavage of catechol (FIG. 1) (5, 6, 23), we sought to divert PCA towards this ring-opening pathway. However, conversion of PCA to catechol has been observed as a metabolic bottleneck in other engineered bacterial strains (5, 6). Since the activity of the N. aromaticivorans NadCD enzyme was lower than that of the well-studied PCA decarboxylase EcAroY, we generated two strains which contained either nadBCD (LigAB1_NaDec) or ecdB/ecaroY/ecdD (LigAB1_EcDec) in the ligAB1 (Saro_2812-13) locus of 12444_PCA. We chose to place genes encoding either of the PCA decarboxylase genes in the ligAB1 locus as this locus is highly transcribed when N. aromaticivorans is grown in the presence of G-family aromatic compounds (33). Based on the activity of these two decarboxylases in vitro, we predicted that insertion of these PCA decarboxylase genes into the ligAB1 locus of the 12444_PCA strain should allow conversion of PCA to catechol in vivo and reduce or block extracellular PCA accumulation.

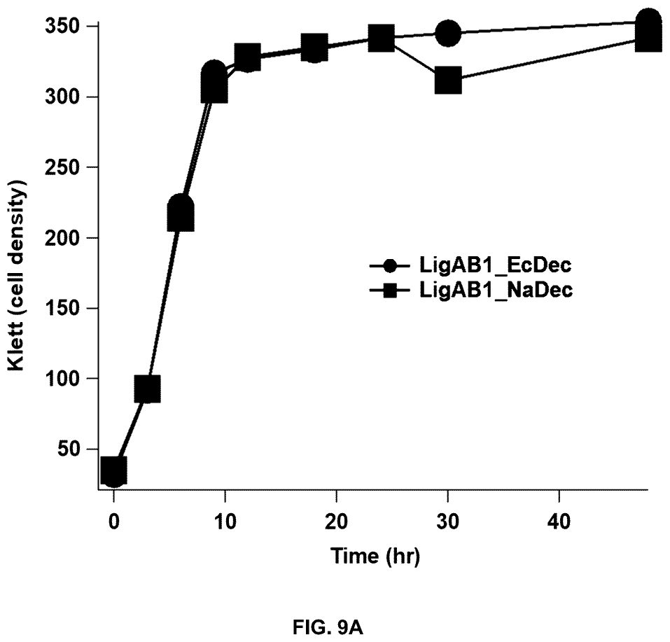

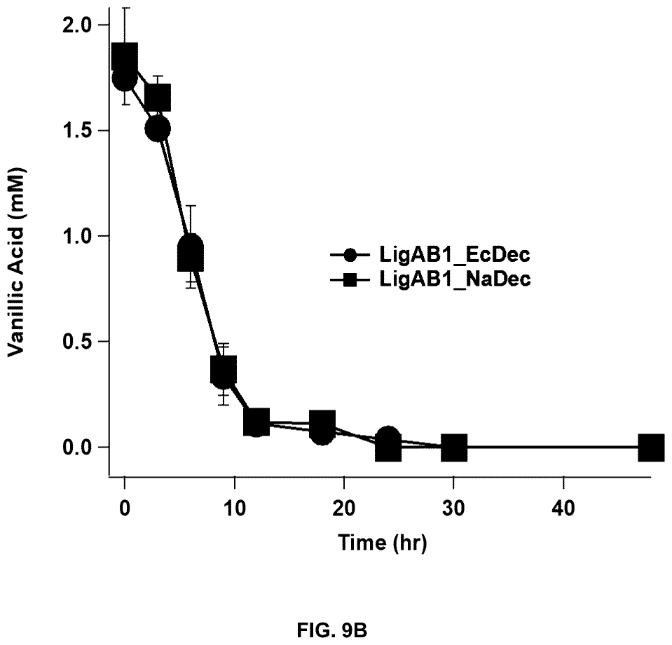

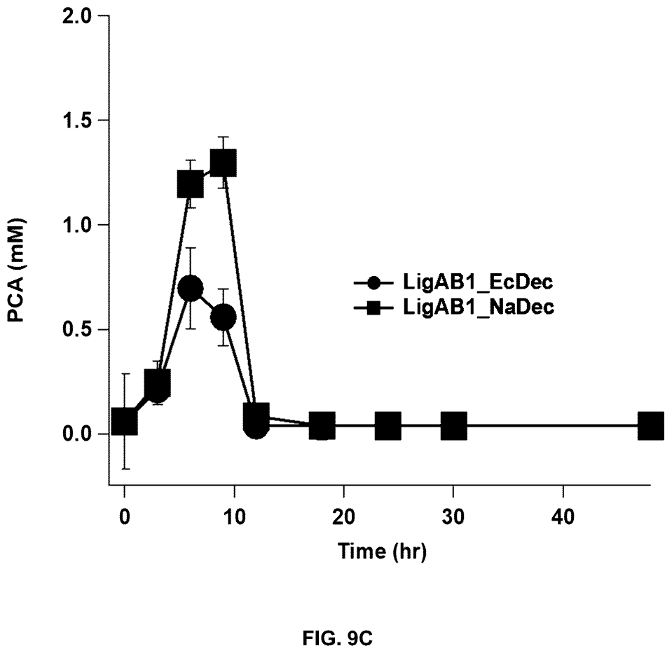

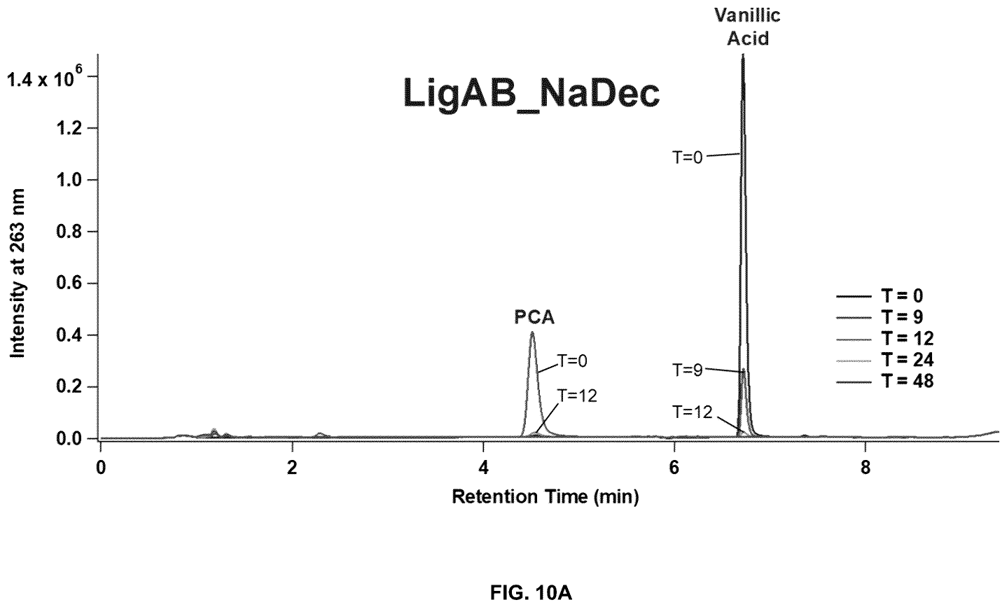

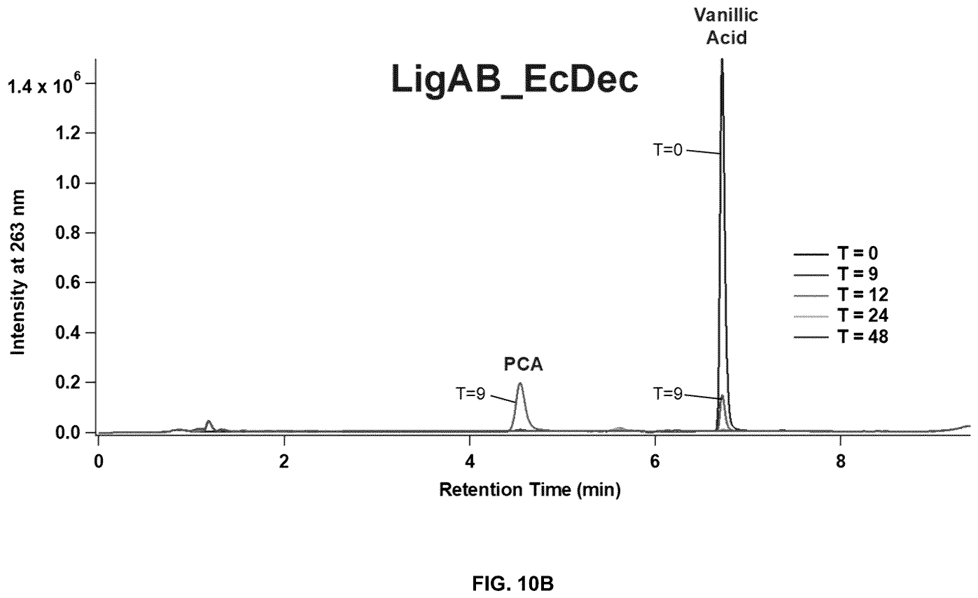

To test this hypothesis, we grew strains LigAB1_EcDec and LigAB1_NaDec in media containing 2 mM vanillic acid and 10 mM glucose as an auxiliary carbon source. We found that both the LigAB1_EcDec and LigAB1_NaDec strains reached similar final cell densities and completely consumed vanillic acid within 12 h (FIGS. 9A and 9B). We observed transient accumulation of extracellular PCA in each strain, reaching a maximum of 1.4±0.1 mM in the LigAB1_NaDec at 10 h and 0.8±0.2 mM at 6 h for LigAB1_EcDec (FIG. 9C). However, extracellular PCA was undetectable in both cultures after 12 h (FIGS. 10A and 10B). These results indicate that placement of either the Ec or Na PCA decarboxylase genes at the ligAB1 locus of the 12444_PCA strain restored the strain's ability to assimilate this pathway intermediate. In comparison to the 12444_ΔligAB1/2 strain, which has the genes for nadBCD in its native locus, both of the above strains consume PCA faster with full consumption of this pathway intermediate within 12 h compared to 24 h. This is consistent with the observation of higher transcript levels from the ligAB1 locus versus the nadBCD locus when cells are grown in the presence of G-family aromatics (33).

N. aromaticivorans Genes Predicted to be Involved in Catechol Metabolism

While both LigAB1_NaDec and LigAB1_EcDec were able to metabolize PCA, neither of these strains accumulated detectable levels of extracellular catechol or ccMA (FIGS. 10A and 10B). To explain this observation, we propose that these strains contain one or more previously undescribed pathways for catechol assimilation into cellular material. Therefore, we sought to identify potential enzymes involved in catechol metabolism in N. aromaticivorans.

Bacterial catechol catabolism can be initiated via extradiol cleavage by a 2,3 dioxygenase, XylE, producing 2-hydroxymuconate semialdehyde or through intradiol cleavage by a 1,2-catechol dioxygenase, CatA, to generate ccMA (FIG. 1). To predict the potential N. aromaticivorans pathways for catechol metabolism, we analyzed its genome for homologs of genes that encode enzymes that function in the extradiol and intradiol pathways. This analysis predicted the presence of catBCA and xylEGHIJKQ (26) transcription units that encode N. aromaticivorans proteins with >40% amino acid identity to known enzymes in the intradiol and extradiol cleavage pathways, respectively (Tables 1 and 11), suggesting that N. aromaticivorans can potentially metabolize catechol by both pathways.

TABLE 11
The amino acid sequence identity of the CatBCA from Pseudomonas putida
or Enterobacter cloacae compared to homologues encoded by
Novosphingobium aromaticivorans.

| | Saro_3828 | Saro_3829 | Saro_3830 |
|---|---|---|---|
| Amino Acid Sequence Identity | (NaCatB) | (NaCatC) | (NaCatA) |
| Enterobacter cloacae (EcCatB) | 63% | | |
| Pseudomonas putida (PpCatB) | 62% | | |
| Enterobacter cloacae (EcCatC) | | 69% | |
| Pseudomonas putida (PpCatC) | | 60% | |
| Enterobacter cloacae (EcCatA) | | | 43% |
| Pseudomonas putida (PpCatA) | | | 49% |

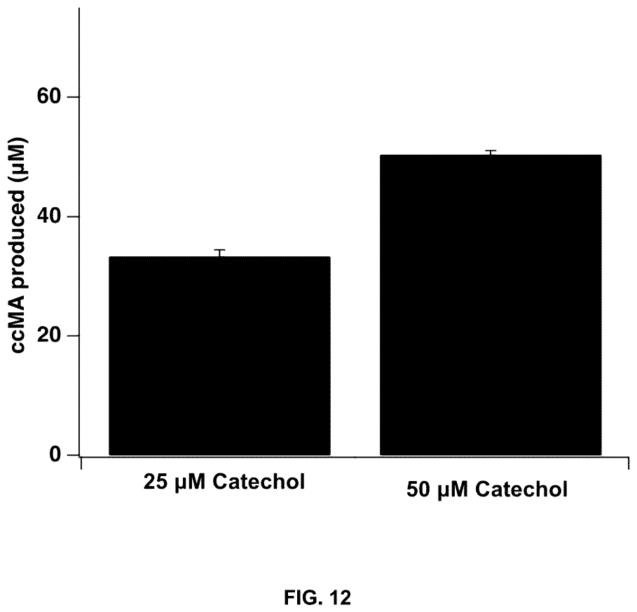

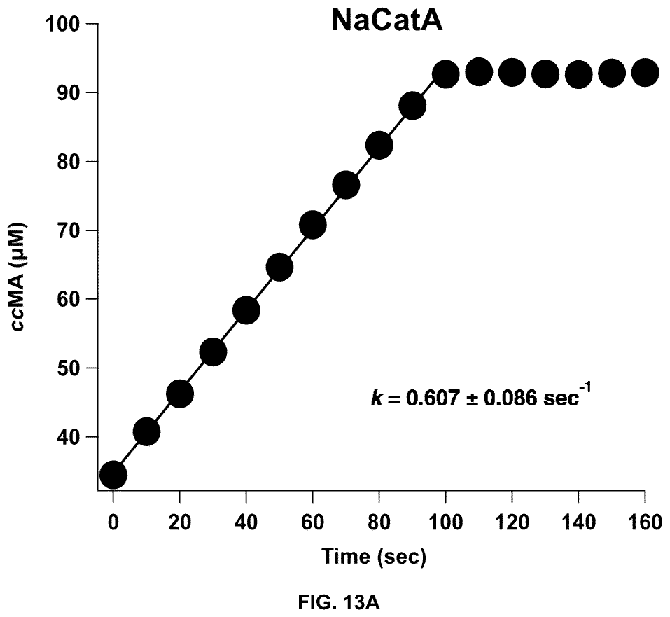

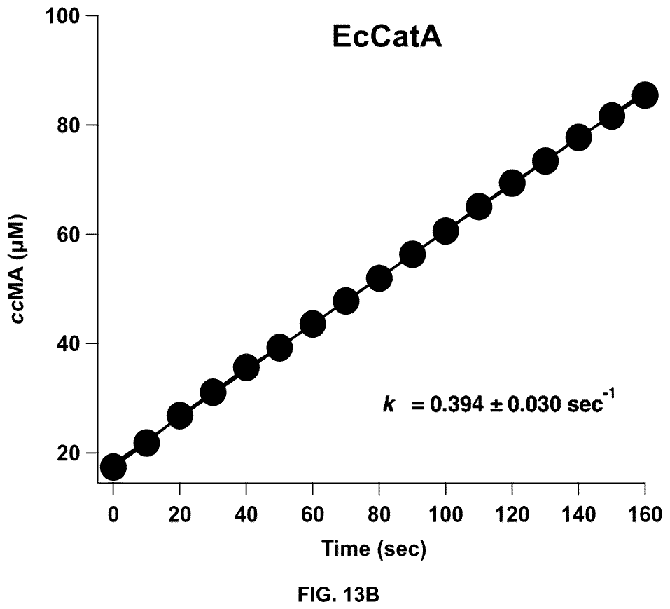

Of these two potential catechol cleavage pathways, only CatA is predicted to generate ccMA. Unlike some other aromatic metabolizing bacteria (49), the N. aromaticivorans genome is not predicted to contain a second copy of a gene that encodes a protein with amino acid sequence identity to known CatA enzymes (Table 11). Thus, we sought to test the activity of the predicted, but previously uncharacterized N. aromaticivorans CatA enzyme (NaCatA), and to compare it to the CatA of E. cloacae (EcCatA). To do this, the catechol 1,2 dioxygenase activity of purified recombinant NaCatA and EcCatA proteins (FIGS. 11A and 11B) was monitored by following the absorbance at 260 nm corresponding to ccMA by UV/visible absorption spectroscopy (50). Under identical assay conditions, both EcCatA and NaCatA produced stoichiometric (FIG. 12) amounts of ccMA and the data best fit to a linear equation yielding zeroth order rate constants on the same order of magnitude of each other (FIGS. 13A and 13B). From this we conclude that NaCatA catalyzes the intradiol cleavage of catechol to produce ccMA in vitro and exhibits a similar rate of activity as EcCatA under these assay conditions.

Engineering a N. aromaticivorans Strain to Divert Catechol to ccMA

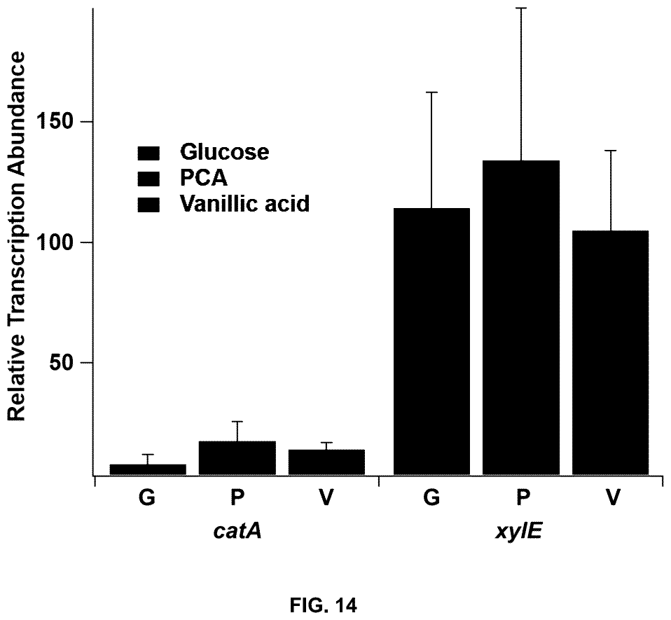

Based on the genomic, bioinformatic and in vitro analysis of N. aromaticivorans enzymes that are predicted to be involved in catechol metabolism, we reasoned that several genetic modifications could be employed to engineer a strain that accumulated extracellular ccMA from pathway intermediates like PCA. First, the existence of a catBCA operon for intradiol cleavage of catechol predicted deletion of catBC genes would block metabolism of the ccMA generated by CatA activity. To do this, we generated ΔcatBC derivatives of strains LigAB1_NaDec and LigAB1_EcDec, which produced the strains, NaDec_cat and EcDec_cat respectively (Table 3). We also reasoned that inactivation of the xylE-dependent pathway for extradiol cleavage of catechol would divert catechol through the intradiol CatA-dependent pathway. Therefore, we replaced the native N. aromaticivorans xylE with either eccatA or the nacatA producing EcDec_ccMA or NaDec_ccMA, respectively (Table 3). We inserted catA into the native xylE locus since the transcript abundance of the xylE gene in N. aromaticivorans is higher than catA transcript levels when wild-type cells are grown in the presence of aromatics (FIG. 14) (33). We reasoned that placement of eccatA or nacatA at the xylE locus should help ensure that these engineered strains were expressing sufficient levels of CatA to completely metabolize catechol to ccMA. We chose to generate strains expressing either EcCatA or NaCatA from the same locus in order to test whether the two versions of CatA led to production of significantly different levels of extracellular ccMA.

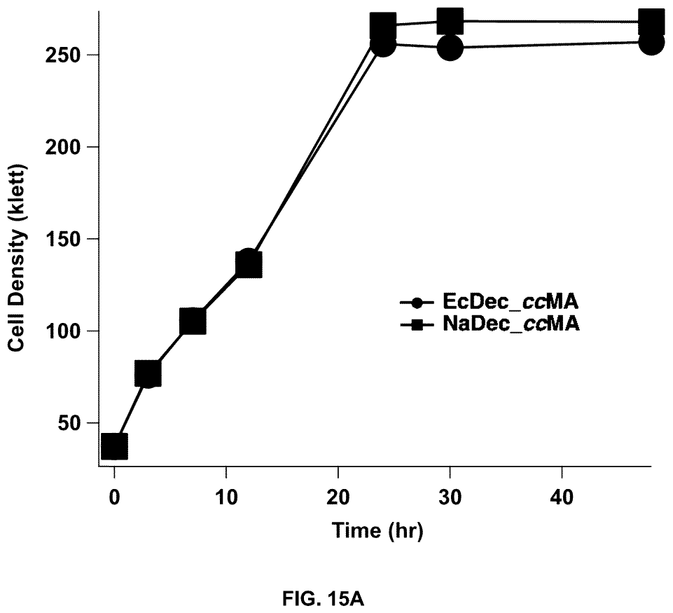

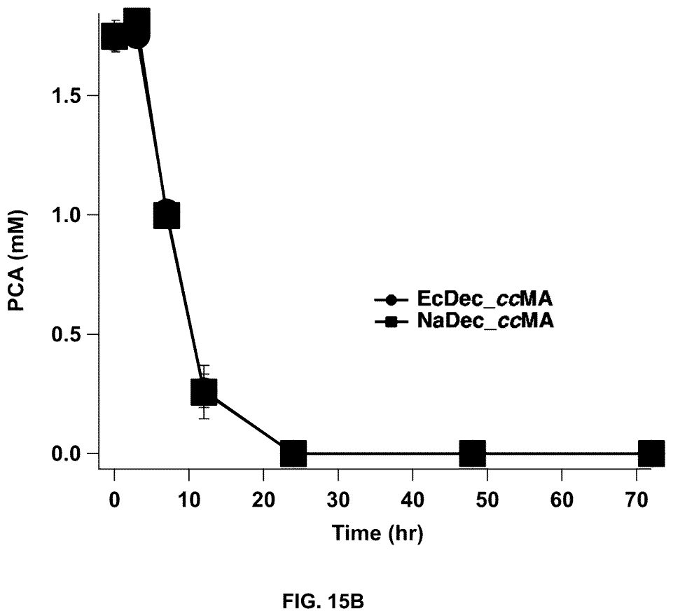

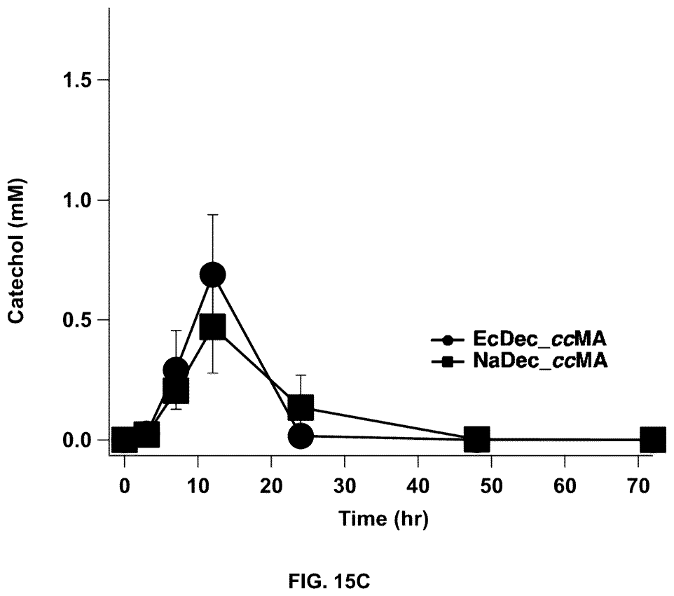

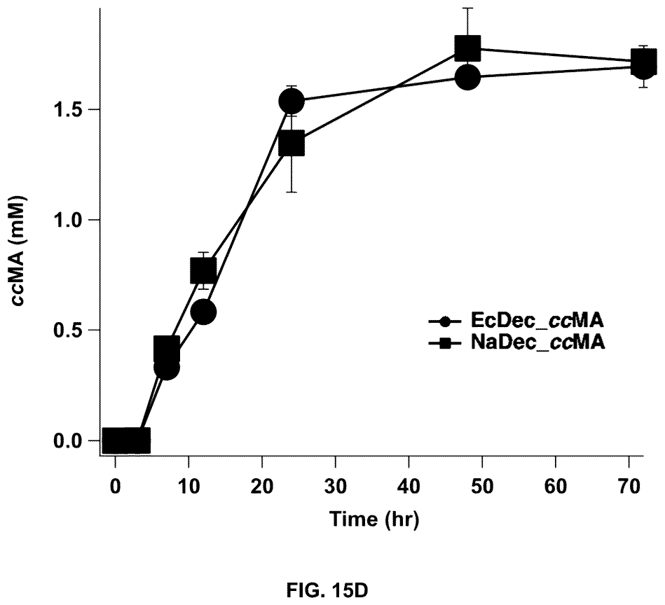

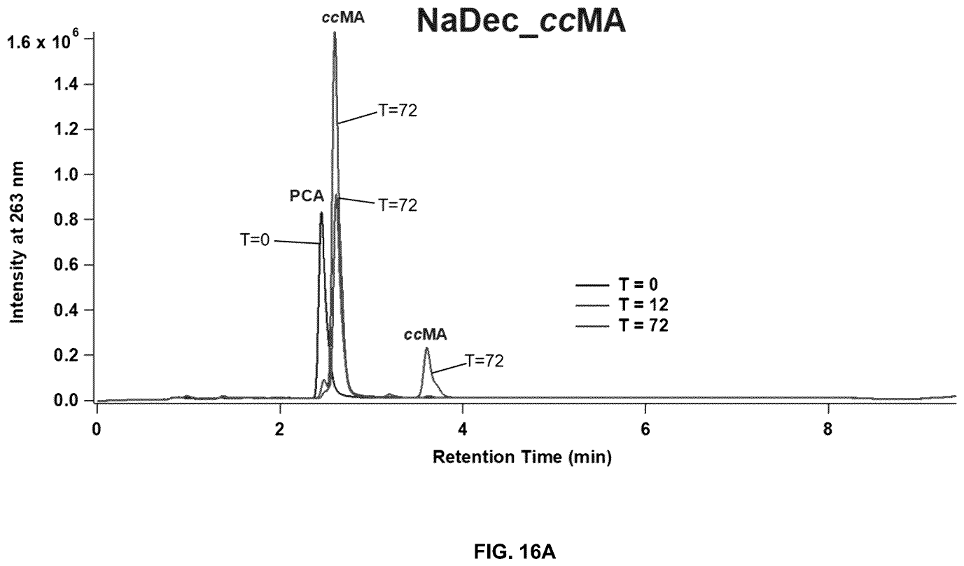

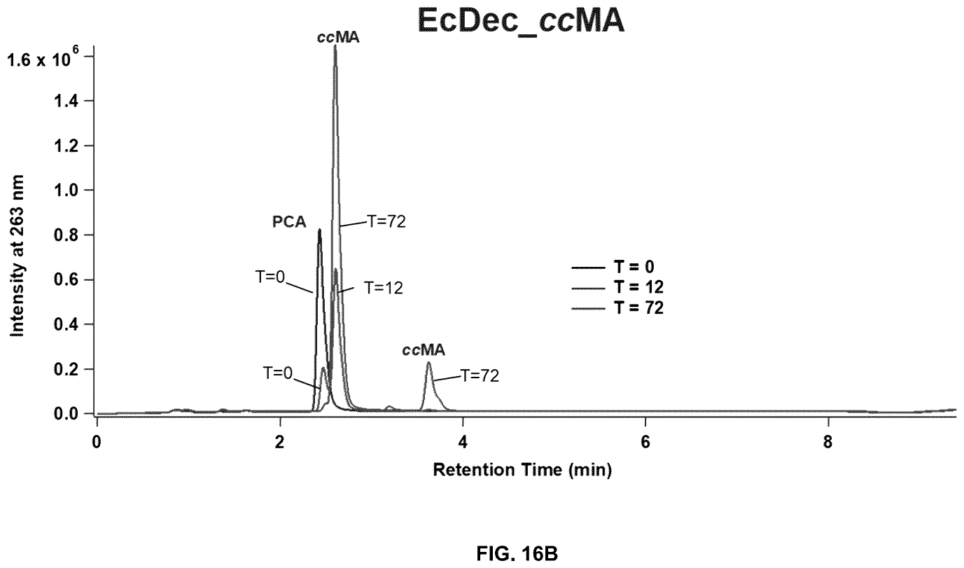

To test the impact of these genomic alterations on aromatic metabolism, we evaluated the ability of EcDec_ccMA and NaDec_ccMA to convert PCA into ccMA by growing these strains in media containing 2 mM PCA and 10 mM glucose as an auxiliary carbon source. Both strains exhibited transient accumulation of catechol (FIG. 15C) and completely converted PCA to ccMA within 30 h with ccMA yields of 100±5% for NaDec_ccMA or 97±3% for EcDec_ccMA (FIGS. 15B, 15D, 16A and 16B). These results illustrate that these alterations to the genome of N. aromaticivorans allowed for funneling of PCA to the catechol intradiol branch and subsequent ccMA production. This finding provides new evidence in support that N. aromaticivorans can be used as a chassis for the metabolic conversion of G and H aromatics into ccMA.

Synthesis of ccMA from Aromatics in Poplar APL

To further evaluate the use of N. aromaticivorans as a chassis for ccMA production, we tested the ability of the NaDec_ccMA and EcDec_ccMA strains to produce this compound from biomass-derived aromatics. It is known that transgenic plants expressing the quinate and shikimate utilization B (qsuB) gene increase the accumulation of aromatics, notably PCA, found in biomass (51). Previously we have shown that these QsuB transgenic poplar plants can be used as a source of aromatics for the conversion of biomass aromatics to PDC (52). Thus, we tested the ability of the NaDec_ccMA and EcDec_ccMA strains to produce ccMA from aromatics derived from a transgenic poplar QsuB plant.

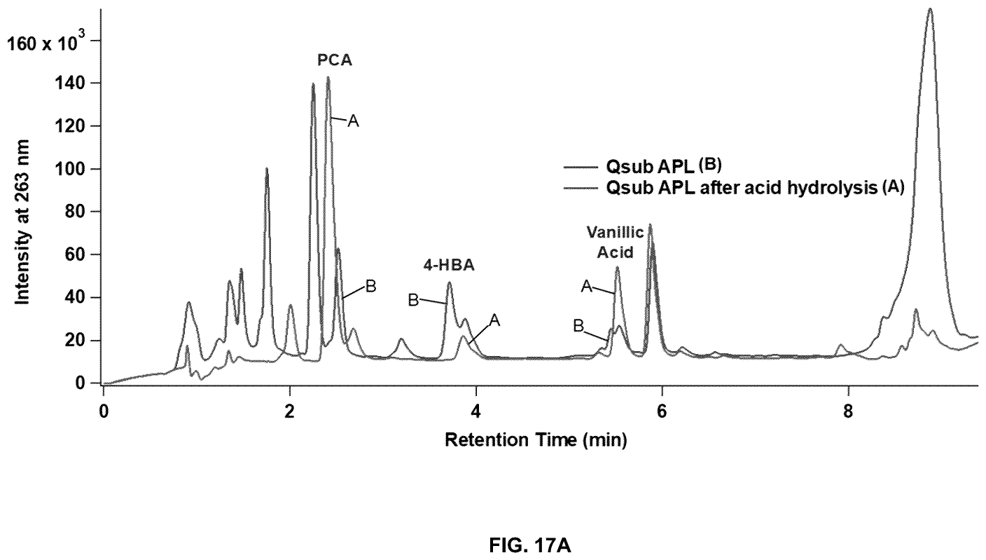

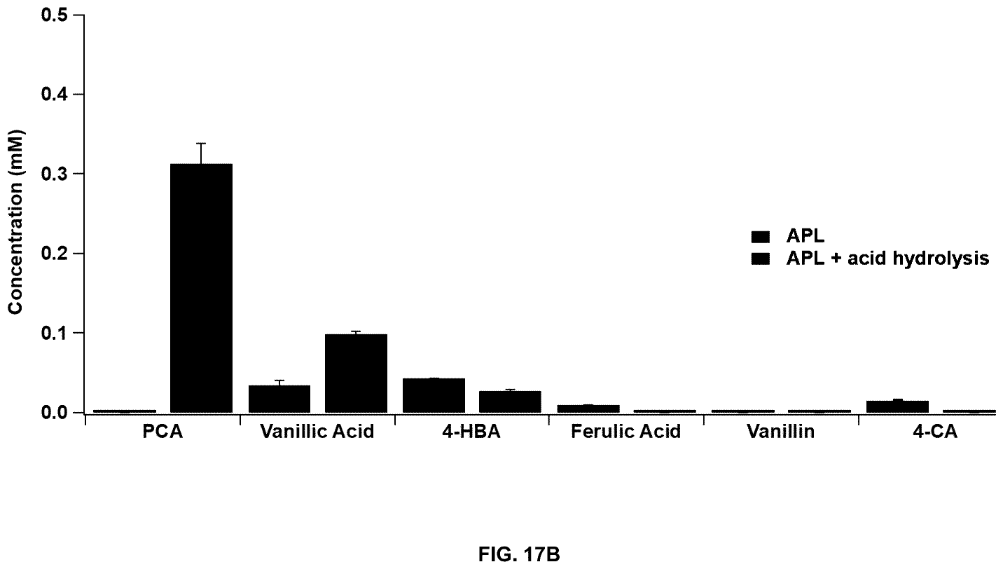

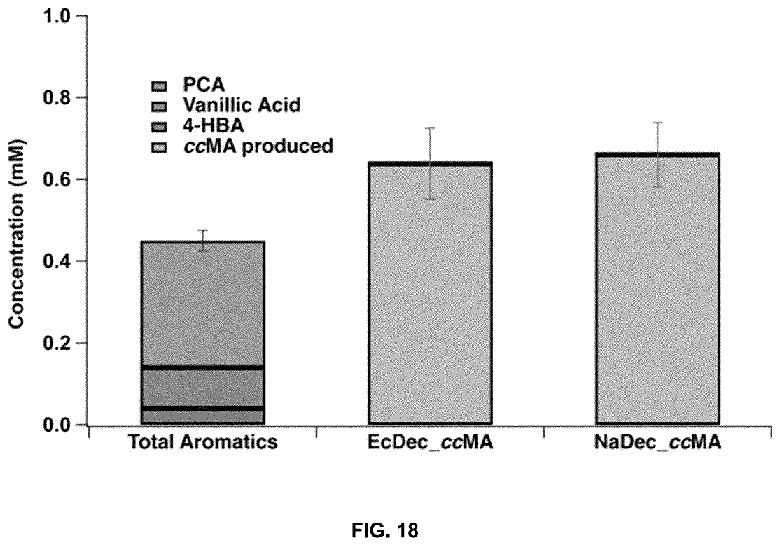

An aqueous solution containing both phenolic monomers and glycosylated forms of PCA and vanillic acid was obtained from QsuB poplar biomass using a mild alkaline pretreatment that cleaves ester linkages (FIGS. 17A and 17B) (52). After acid treatment of the alkaline pretreatment liquor (APL) to release the glycosylated phenolic compounds (52), the major identifiable phenolics in this material were PCA, vanillic acid, and 4-hydroxybenzoic acid (4-HBA) with trace amounts of ferulic acid and 4-coumaric acid (Table 12). N. aromaticivorans is able to metabolize both free and glycosylated phenolics (52), so the concentration of known phenolics derived from the Qsub poplar biomass was calculated to include the concentration of the glycosylated phenolics (52). This calculation showed that we obtained 0.42±0.03 mM of total aromatics in the Qsub APL with PCA accounting approximately 60% of the total aromatics (FIGS. 18, 17A, and 17B).

TABLE 12
Concentration of aromatics in Qsub Poplar APL before and after acid
hydrolysis. ND represents that the phenolic was not detected and
trace indicates that a compound that co-migrated with a commercial
standard was detected but at too low of levels to be quantified.

| Aromatic | Before acid hydrolysis | After acid hydrolysis |
|---|---|---|
| PCA | 0.00 ± 0.000 mM | 0.31 ± 0.025 mM |
| Vanillic Acid | 0.04 ± 0.006 mM | 0.10 ± 0.028 mM |
| 4-HBA | 0.04 ± 0.001 mM | 0.03 ± 0.014 mM |
| Vanillin | ND | ND |
| 4-CA | Trace | ND |
| Ferulic Acid | Trace | ND |

To test for microbial production of ccMA from the aromatics in QsuB poplar APL, we added glucose as an auxiliary carbon source and ammonium sulfate as a nitrogen source to cultures of EcDec_ccMA or NaDec_ccMA. After 48 h, both strains produced ccMA from QsuB poplar APL with calculated yields of 157±26% for EcDec_ccMA and 163±25% NaDec_ccMA (FIG. 18). After 48 h of incubation, we did not observe any 4-HBA or PCA in the medium and only trace amounts of vanillic acid (FIGS. 17A and 17B), which indicates that these APL aromatics were metabolized and possibly converted to ccMA. From this, we conclude that the EcDec_ccMA and NaDec_ccMA strains were both capable of production of ccMA from Qsub APL poplar. Possible explanations for the greater than 100% yield of ccMA from components of the Qsub poplar APL are provided in the discussion.

### Discussion

Lignin is the second most abundant renewable polymer on Earth (8) and represents a potential source of phenolics for conversion into industrial chemicals and materials (12). Despite this, the heterogeneity of aromatic monomers and their inter-subunit linkages have presented challenges in producing sources of valuable chemicals from this abundant resource. In recent years, the ability of some microbes to funnel a diverse set of aromatics to common intermediates has catalyzed interest in using genome-enabled strain engineering to generate one or more valuable compounds from these phenolic mixtures (7, 15, 37). This study investigated the utility of the aromatic metabolizing bacterium N. aromaticivorans as a chassis for ccMA production from biomass aromatics by combining an expanded knowledge base of its aromatic metabolic pathways with metabolic engineering.

Microbial production of ccMA from glucose has been reported in bacteria that either lack or have a limited ability to metabolize the mixed aromatics that are abundant in plant biomass (53, 54). Many efforts to produce ccMA from aromatics have been limited by the accumulation of pathway intermediates (6, 22-24). We chose N. aromaticivorans as a potential host for ccMA production as it has the native ability to metabolize the major aromatic monomers found in plant cells walls, to transport and cleave low molecular weight aromatic oligomers with different inter-subunit linkages, and to convert modified phenolics that are formed by some methods of biomass or lignin deconstruction (11, 30, 55). Below, we summarize how we combined bioinformatics, enzymology, and the genetic tractability of N. aromaticivorans to gain knowledge about its pathways for aromatic metabolism and use this information to engineer strains that produce ccMA from aromatics present in biomass.

### Diverting PCA to Catechol

Previous studies that engineered N. aromaticivorans to produce PDC from aromatics suggested that the extradiol cleavage of PCA by 4,5 PCA dioxygenase homologs (LigAB1 and LigAB2) represented a major route for metabolism of this pathway intermediate (31, 32). However, the results of these studies also predicted the possibility of another minor route for PCA metabolism since yields of PDC were significantly less than 100% when a PDC-producing strain was grown in the presence of PCA (31, 32). Furthermore, work with a derivative of the PDC producing strain that had deletions of both the ligAB1 and ligAB2 gene sets found that, when grown with vanillic acid, the cells accumulated only ˜50% of expected PCA and failed to accumulate PDC (32). Combined, these results suggest that LigAB1 and LigAB2 both play a significant role in PCA metabolism but that another PCA metabolic pathway exists. In this study, we identified a previously uncharacterized PCA decarboxylase as the enzyme that is responsible for consumption of PCA in cells that lack the two 4,5 PCA dioxygenase homologs (LigAB1 and LigAB2).

As predicted from previous work (31), we found that inactivation of both LigAB1 and LigAB2 in the 12444_ΔligAB1/2 strain resulted in only transient PCA accumulation (FIG. 4C), leading us to conclude that this mutant was able to consume PCA by another catabolic pathway. Analysis of both the N. aromaticivorans genome sequence and of the transcript levels when cells are grown in the presence of G-family aromatics suggested that a previously uncharacterized PCA decarboxylase could be converting PCA to catechol when genes encoding both LigAB homologues were deleted. In addition, a published analysis of a genome-wide transposon insertion library suggested a connection between PCA and catechol metabolism in N. aromaticivorans (26). Our genetic data and in vitro enzyme assays of the previously uncharacterized N. aromaticivorans NadCD led us to propose that NadCD is the PCA decarboxylase that is responsible for conversion of PCA to catechol (FIG. 1). As predicted by this hypothesis, we found that deletion of the genes encoding NadCB in the 12444_ΔligAB1/2 strain resulted in the accumulation of extracellular PCA. We think it is unlikely that the less than 100% PCA recovery (78±5%) in this strain reflects the function of another unknown PCA catabolic pathway in N. aromaticivorans. In support of this hypothesis, we could not identify genes that encode proteins with >25% amino acid identity to the intradiol 3,4 or to the extradiol 2,3 PCA dioxygenases found in other reported PCA catabolic pathways (35-37). Therefore, we suggest that the lack of 100% stoichiometry of PCA in these strains is due to the well-known abiotic oxidation of this compound (46-48). Instead, our data suggests that a heretofore uncharacterized N. aromaticivorans decarboxylase (NadCD) is responsible for PCA metabolism through conversion to catechol.

The prediction that N. aromaticivorans contains a PCA decarboxylase (NadCD) was not expected as there are only a few characterized homologs of this enzyme in aromatic-metabolizing bacteria (39, 45, 56). Indeed, while most metabolic engineering strategies for ccMA production using aromatic-metabolizing bacteria take advantage of the intradiol cleavage pathway of aromatic metabolism for ccMA production, they often utilize a PCA decarboxylase from another bacterium (often E. cloacae or K. pneumoniae) to convert PCA into catechol (4, 20, 57). In addition, the conversion of PCA to catechol is often a bottleneck in ccMA production; one that has been circumvented in P. putida KT2440 by using a foreign promoter to increase expression of a foreign PCA decarboxylase that is comprised of the EcdB/EcAroY/EcdD proteins. For these reasons, we sought to compare the PCA decarboxylase activity of NadCD to that of the commonly used EcAroY/EcdD decarboxylase.

Our in vitro analysis of recombinant NadCD and EcAroY showed that under identical conditions, the N. aromaticivorans enzyme was active, albeit slower, than the E. cloacae homologue. The lower activity of NadCD compared to EcAroY in vitro suggested that the conversion of PCA to catechol in vivo could be slower for engineered strains that depend on the native PCA decarboxylase. Indeed, the LigAB1_NaDec strain showed a higher transient extracellular level of PCA in vivo than the strain containing genes encoding the E. cloacae decarboxylase at the same locus (LigAB1_EcDec). However, a more detailed analysis of enzyme activity in vitro is needed to confirm this correlation. Nevertheless, only transient accumulation of PCA was found when either LigAB1_EcDec or LigAB1_NaDec are grown with vanillic acid, indicating active PCA decarboxylation in either strain and full consumption of PCA by the end of the experiment.

This study also demonstrated that the insertion of the nadBCD genes into the ligAB1 locus resulted in faster PCA consumption as compared to the 12444_ΔligAB1/2 strain, which has nadBCD in its native locus. The faster PCA consumption observed in the LigAB1_NaDec strain is consistent with the previous transcript analysis of N. aromaticivorans that showed higher abundance of ligAB1 transcripts than those from nadBCD when cells are grown in the presence of G-family aromatics (33). These results demonstrated that placement of either naBCD or ecdB/ecaroY/ecdD into the ligAB1 locus provided sufficient decarboxylase activity for PCA consumption. From this, we conclude that the predicted PCA decarboxylase transcript levels in the LigAB1_NaDec strain are higher than those which express the PCA decarboxylase only from its native locus and contributes to faster PCA decarboxylation by this strain in vivo.

Diverting Catechol to ccMA

Previous analysis of N. aromaticivorans indicated high transcript levels of the catechol 2,3-dioxygenase (xylE) when cells are grown in the presence of PCA or one of several G-family aromatics (33). These results suggest that catechol can be metabolized through the extradiol pathway (FIG. 1). However, evidence was lacking for the function of a CatA homologue in the intradiol cleavage pathway of catechol to ccMA in this bacterium. While the N. aromaticivorans genome encodes a protein with significant amino acid sequence identity to other CatA enzymes (Table 11), nacatA transcripts are lower than those encoding enzymes of other known aromatic metabolizing enzymes when cells are grown in the presence of G-family aromatics (33). There is also no published evidence for metabolism of catechol via the intradiol branch in N. aromaticivorans. Therefore, to generate additional knowledge of N. aromaticivorans aromatic metabolism we tested a purified recombinant NaCatA for catechol 1,2-dioxygenase activity.

Our in vitro results indicated that recombinant NaCatA was active for catechol 1,2-dioxygenase activity and that it has comparable activity to recombinant EcCatA enzyme. Other catechol 1,2-dioxygenases typically follow Michaelis-Menten kinetics (58), so the zeroth order plot obtained with either CatA from N. aromaticivorans or E. cloacae suggest substrate saturation by catechol of both enzymes. While further kinetic analysis of NaCatA is needed to confirm that both enzymes exhibit typical Michaelis-Menten kinetics, our results suggest that N. aromativorans encodes a catechol 1,2-dioxygenase that is capable of converting catechol to ccMA. Overall, the results of these experiments are the first report that N. aromativorans has the ability to metabolize aromatics via the intradiol branch of catechol catabolism.

ccMA Production from Biomass Aromatics by N. aromaticivorans

Comparison between the transcript abundance of catA and xylE suggested that the extradiol (xylE) dependent pathway is the major pathway for catechol catabolism in N. aromaticivorans. The relatively low catA transcript abundance when cells were grown in the presence of aromatics suggested there might be little to no flux through this intradiol CatA-dependent pathway in these cultures. Additionally, the presence of only one gene encoding a protein with amino acid sequence similarity to known CatA enzymes, suggested there was a potential bottleneck in the conversion of catechol to ccMA when relying on the expression of catA from its native locus. Therefore, the genes for nacatA and eccatA were separately placed into the xylE locus, a region that is highly transcribed when cells are grown in the presence of aromatics (33). We also inactivated xylE and catBC in order to generate a strain which is predicted to only metabolize catechol via the intradiol pathway and unable to metabolize ccMA. This generated two N. aromaticivorans strains to compare for ccMA accumulation using genes derived from either N. aromaticivorans (NaDec_ccMA) or E. cloacae (EcDec_ccMA). As predicted by the similar rates observed in vitro for EcCatA or NaCatA, when cells were grown with PCA, both strains accumulated minimal amounts of catechol and produced stoichiometric ccMA at a similar rate. Stoichiometric conversion of ccMA from PCA indicated that the knowledge gained from these experiments on the aromatic metabolism of N. aromaticivorans was successfully implemented to direct PCA metabolism to the catechol intradiol pathway using either native genes or genes derived from E. cloacae.

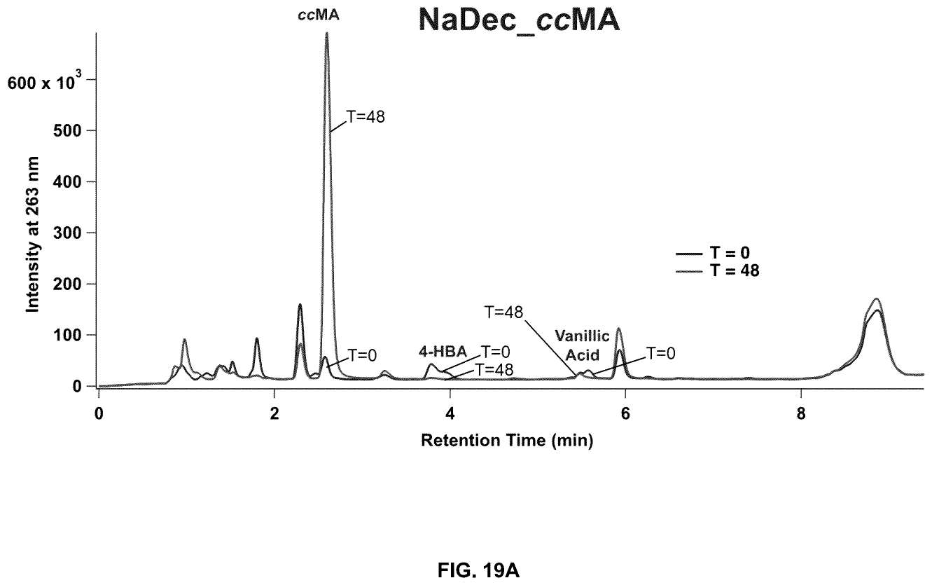

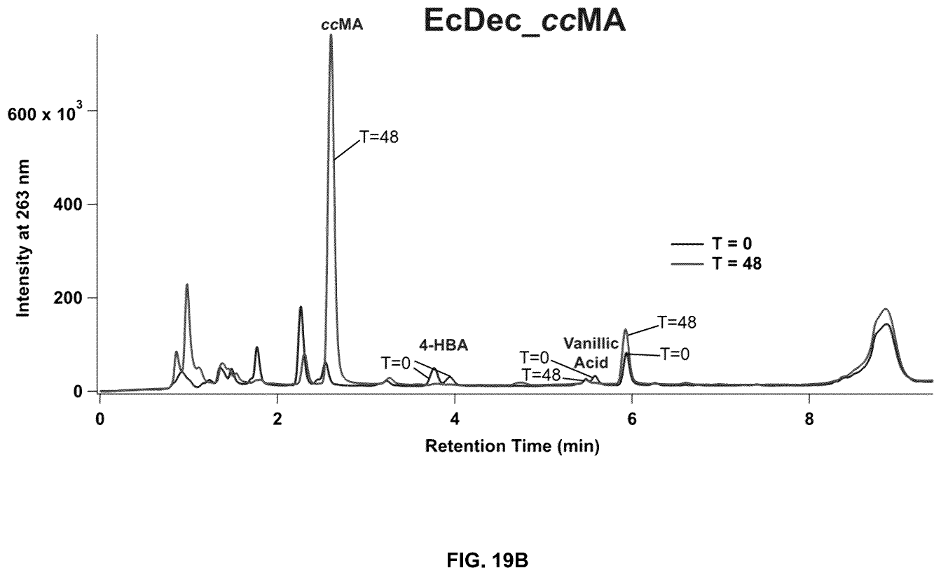

The industrial conversion of abundant renewable aromatics by metabolically engineered microorganisms requires the ability of these strains to generate commodity chemicals from deconstructed biomass. To date, strains tested for ccMA production from crude biomass aromatics produced ccMA yields ranging from 5-100%, with most strains producing less than 50% yields of ccMA from deconstructed lignin (22). To test the feasibility of our engineered N. aromaticivorans strains to produce ccMA from biomass aromatics, we chose to use QsuB poplar biomass (51) because (1) our strains were engineered to use PCA as a precursor for ccMA production and (2) our results showed they were capable of stoichiometric conversion of PCA to ccMA. Therefore, we predicted that when our strains grow with this APL source, we would get high yields of ccMA. Indeed, at the end of the culture period, LCMS analysis showed one major peak corresponding to ccMA in the extracellular media from the NaDec_ccMA and EcDec_ccMA cultures (FIGS. 19A and 19B). The aromatics that were quantified in the APL included PCA, vanillic acid, and 4-HBA (FIGS. 17A and 17B), with the majority of PCA and vanillic acid found in glycosylated forms. All the PCA and 4-HBA were transformed during incubation, with only trace amounts of vanillic acid remaining after the incubation. Umana et al. demonstrated that the glycosylated forms of PCA and vanillic acid are degraded by N. aromaticivorans, and as PCA is an intermediate in the degradation of vanillic acid and 4-HBA, this observation leads us to conclude that these strains were producing ccMA from all the aromatics quantified in the biomass APL. However, the greater than 100% ccMA yield in the NaDec_ccMA and EcDec_ccMA cultures grown in the presence of APL suggests that both of these engineered strains are capable of metabolizing other unidentified aromatics present in the APL. Furthermore, since the mild alkaline pretreatment process used to generate the APL cleaves the ester bonds and liberates soluble metabolites without breaking down the lignin backbone (52), it is likely that these unidentified aromatics are not oligomeric forms of deconstructed lignin. Nevertheless, the diversity of aromatic catabolic pathways available in N. aromaticivorans is a valuable characteristic of N. aromaticivorans and important for host selection as many lignocellulosic biomass deconstruction methods are more aggressive than the mild alkaline pretreatment used in this study and would produce a range of aromatic monomers, dimers and oligomers, as well as sugar conjugates and other organic materials that could be used to support growth of engineered strains.

Overall, this work increases our knowledge on the diversity of aromatic metabolic routes available in N. aromaticivorans. We identified unreported N. aromaticivorans metabolic pathways that are involved in the conversion of PCA to ccMA. In vitro characterization of newly-identified PCA decarboxylase (NadCD) and catechol 1,2-dioxygenase (NaCatA) enzymes predicted the existence of formerly unknown metabolic routes for aromatic metabolism. We confirmed the function of these metabolic pathways through creation of defined mutants that demonstrated a new route for PCA catabolism to catechol, as well as the function of an intradiol pathway for catechol metabolism in N. aromaticivorans. The existence of a native PCA decarboxylase in N. aromaticivorans is somewhat unique in comparison to other reported ccMA producing hosts which do not naturally possess a PCA decarboxylase capable of converting PCA to catechol (22). The pathways for PCA catabolism in N. aromaticivorans are also different in comparison to other sphingomonads such as Sphingobium sp. SYK-6, since we were unable to identify genes in this well-studied aromatic metabolizing bacterium that encode proteins with significant amino acid sequence identity to known PCA decarboxylases (38). In addition, while we could identify a Sphingobium sp. SYK-6 catA homologue that encoded a protein with ˜40% amino acid sequence identity to E. cloacae CatA, the genome is not predicted to encode proteins with amino acid sequence identity to the typical CatBC enzymes of the catechol intradiol catabolic pathway. These observations increase our knowledge of the number and diversity of N. aromaticivorans aromatic catabolic pathways and further highlight the potential of this bacterium as a host for converting aromatics into commodity chemicals.

Our biochemical and genetic characterization of previously uncharacterized N. aromaticivorans gene products allowed for the generation of an engineered ccMA-producing microbe that is completely derived from native genes and transcriptional units. The use of native enzymes is potentially advantageous as it likely bypasses problems associated with folding or stability of foreign proteins, availability of required cofactors, and the accumulation of unusual intermediates that are part of a pathway that is not normally used by the host. In addition, we found that the N. aromaticivorans PCA decarboxylase and CatA proteins have activity that is comparable to that of well-studied enzymes from other hosts that have been used to build other ccMA production strains. Indeed, comparison of NaDec_ccMA and EcDec_ccMA demonstrated that both strains produced ccMA at similar yields and rates from either pure aromatics or biomass-derived aromatics. These results suggest that it will be possible to further engineer N. aromaticivorans strains for improved ccMA productivity using solely native genes.

In conclusion, our findings have expanded the knowledge of aromatic catabolic pathways in N. aromaticivorans and demonstrated the utility of this bacterium as a chassis for ccMA production from phenolic mixtures derived from lignocellulosic biomass. Our studies provide a proof of concept for stoichiometric ccMA production from N. aromaticivorans and generates a host that can be used for future studies to optimize ccMA production rates, titers and yields in bioreactors. The new findings reported herein also illustrate the value of the genetic and metabolic tractability of the abundant aromatic catabolism pathways in N. aromaticivorans as engineering of these ccMA-producing strains did not require the use of synthetic promoters and additional genomic alterations to produce stoichiometric yields of ccMA from deconstructed biomass. Overall, this work provides new insights in the aromatic metabolism of N. aromaticivorans and highlights the potential for using this bacterium as a host for producing additional valuable products from biomass aromatics.

### REFERENCES

1. Shanks B H, Keeling P L. 2017. Bioprivileged molecules: Creating value from biomass. Green Chem 19:3177-3185.
2. Choi S, Lee H N, Park E, Lee S J, Kim E S. 2020. Recent advances in microbial production of cis,cis-muconic acid. Biomolecules. MDPI AG https://doi.org/10.3390/biom10091238.
3. Wu W, Dutta T, Varman A M, Eudes A, Manalansan B, Loqué D, Singh S. 2017. Lignin Valorization: Two Hybrid Biochemical Routes for the Conversion of Polymeric Lignin into Value-added Chemicals. Sci Rep 7:1-13.
4. Sonoki T, Takahashi K, Sugita H, Hatamura M, Azuma Y, Sato T, Suzuki S, Kamimura N, Masai E. 2018. Glucose-Free cis,cis-Muconic Acid Production via New Metabolic Designs Corresponding to the Heterogeneity of Lignin. ACS Sustain Chem Eng 6:1256-1264.
5. Sonoki T, Morooka M, Sakamoto K, Otsuka Y, Nakamura M, Jellison J, Goodell B. 2014. Enhancement of protocatechuate decarboxylase activity for the effective production of muconate from lignin-related aromatic compounds. J Biotechnol 192:71-77.
6. Johnson C W, Salvachda D, Khanna P, Smith H, Peterson D J, Beckham G T. 2016. Enhancing muconic acid production from glucose and lignin-derived aromatic compounds via increased protocatechuate decarboxylase activity. Metab Eng Commun 3:111-119.
7. Chen Z, Wan C. 2017. Biological valorization strategies for converting lignin into fuels and chemicals. Renew Sustain Energy Rev 73:610-621.
8. Ragauskas A J, Beckham G T, Biddy M J, Chandra R, Chen F, Davis M F, Davison B H, Dixon R A, Gilna P, Keller M, Langan P, Naskar A K, Saddler J N, Tschaplinski T J, Tuskan G A, Wyman C E. 2014. Lignin valorization: Improving lignin processing in the biorefinery. Science (80-). American Association for the Advancement of Science https://doi.org/10.1126/science.1246843.
9. Dahmen N, Lewandowski I, Zibek S, Weidtmann A. 2019. Integrated lignocellulosic value chains in a growing bioeconomy: Status quo and perspectives. GCB Bioenergy 11:107-117.
10. Francois J M, Alkim C, Morin N. 2020. Engineering microbial pathways for production of bio-based chemicals from lignocellulosic sugars: Current status and perspectives. Biotechnol Biofuels. BioMed Central https://doi.org/10.1186/s13068-020-01744-6.
11. Presley G N, Werner A Z, Katahira R, Garcia D C, Haugen S J, Ramirez K J, Giannone R J, Beckham G T, Michener J K. 2021. Pathway discovery and engineering for cleavage of a β-1 lignin-derived biaryl compound. Metab Eng 65:1-10.
12. Becker J, Wittmann C. 2019. A field of dreams: Lignin valorization into chemicals, materials, fuels, and health-care products. Biotechnol Adv 37:107360.
13. Rajesh Banu J, Kavitha S, Yukesh Kannah R, Poornima Devi T, Gunasekaran M, Kim S H, Kumar G. 2019. A review on biopolymer production via lignin valorization. Bioresour Technol. Elsevier Ltd https://doi.org/10.1016/j.biortech.2019.121790.
14. Rosini E, Molinari F, Miani D, Pollegioni L. 2023. Lignin Valorization: Production of High Value-Added Compounds by Engineered Microorganisms.
15. Beckham G T, Johnson C W, Karp E M, Salvachda D, Vardon D R. 2016. Opportunities and challenges in biological lignin valorization. Curr Opin Biotechnol 42:40-53.
16. Kamimura N, Takahashi K, Mori K, Araki T, Fujita M, Higuchi Y, Masai E. 2017. Bacterial catabolism of lignin-derived aromatics: New findings in a recent decade: Update on bacterial lignin catabolism. Environ Microbiol Rep 9:679-705.
17. Fuchs G, Boll M, Heider J. 2011. Microbial degradation of aromatic compounds from one strategy to four https://doi.org/10.1038/nrmicro2652.
18. Bleem A, Kuatsjah E, Presley G N, John E, Beckham G T, Michener J K, Bleem A, Kuatsjah E, Presley G N, Hinchen D J, Zahn M, Garcia D C, Michener W E, Ko G, Tornesakis K. 2022. Article Discovery, characterization, and metabolic engineering of Rieske non-heme iron monooxygenases for guaiacol O-demethylation engineering of Rieske non-heme iron monooxygenases for guaiacol O-demethylation 1-23.
19. Shinoda E, Takahashi K, Abe N, Kamimura N, Sonoki T, Masai E. 2019. Isolation of a novel platform bacterium for lignin valorization and its application in glucose-free cis,cis-muconate production. J Ind Microbiol Biotechnol 46:1071-1080.
20. Salvachda D, Johnson C W, Singer C A, Rohrer H, Peterson D J, Black B A, Knapp A, Beckham G T. 2018. Bioprocess development for muconic acid production from aromatic compounds and lignin. Green Chem 20:5007-5019.
21. Vardon D R, Franden M A, Johnson C W, Karp E M, Guarnieri M T, Linger J G, Salm M J, Strathmann T J, Beckham G T. 2015. Adipic acid production from lignin. Energy Environ Sci 8:617-628.
22. Salvachúa D, Johnson C W, Singer C A, Rohrer H, Peterson D J, Black B A, Knapp A, Beckham G T. 2018. Bioprocess development for muconic acid production from aromatic compounds and lignin. Green Chem 20:5007-5019.
23. Johnson C W, Abraham P E, Linger J G, Khanna P, Hettich R L, Beckham G T. 2017. Eliminating a global regulator of carbon catabolite repression enhances the conversion of aromatic lignin monomers to muconate in Pseudomonas putida KT2440. Metab Eng Commun 5:19-25.
24. Kuatsjah E, Johnson C W, Salvachda D, Werner A Z, Zahn M, Szostkiewicz C J, Singer C A, Dominick G, Okekeogbu I, Haugen S J, Woodworth S P, Ramirez K J, Giannone R J, Hettich R L, McGeehan J E, Beckham G T. 2022. Debottlenecking 4-hydroxybenzoate hydroxylation in Pseudomonas putida KT2440 improves muconate productivity from p-coumarate. Metab Eng 70:31-42.
25. Higuchi Y, Kamimura N, Takenami H, Kikuiri Y, Yasuta C, Tanatani K, Shobuda T, Otsuka Y, Nakamura M, Sonoki T, Masai E. 2022. The Catabolic System of Acetovanillone and Acetosyringone in Sphingobium sp. Strain SYK-6 Useful for Upgrading Aromatic Compounds Obtained through Chemical Lignin Depolymerization. Appl Environ Microbiol 88.
26. Cecil J H, Garcia D C, Giannone R J, Michener J K. Rapid, Parallel Identification of Catabolism Pathways of Lignin-Derived Aromatic Compounds in Novosphingobium aromaticivorans. Appl Environ Microbiol. 2018 Oct. 30; 84(22):e01185-18.
27. Fredrickson J K, Balkwill D L, Drake G R, Romine M F, Ringelberg D B, White D C. 1995. Aromatic-degrading Sphingomonas isolates from the deep subsurface. Appl Environ Microbiol 61:1917-1922.
28. Fredrickson J K, Brockman F J, Workman D J, Li S W, Stevens T O. 1991. Isolation and characterization of a subsurface bacterium capable of growth on toluene, naphthalene, and other aromatic compounds. Appl Environ Microbiol 57:796-803.
29. Kontur W S, Olmsted C N, Yusko L M, Niles A V., Walters K A, Beebe E T, Vander Meulen K A, Karlen S D, Gall D L, Noguera D R, Donohue T J. 2019. A heterodimeric glutathione S-transferase that stereospecifically breaks lignin's (R)-aryl ether bond reveals the diversity of bacterial-etherases. J Biol Chem 294:1877-1890.
30. Gall D L, Kontur W S, Lan W, Kim H, Li Y, Ralph J, Donohue T J, Noguera D R. 2018. In vitro enzymatic depolymerization of lignin with release of syringyl, guaiacyl, and tricin units. Appl Environ Microbiol 84.
31. Perez J M, Kontur W S, Alherech M, Coplien J, Karlen S D, Stahl S S, Donohue T J, Noguera D R. 2019. Funneling aromatic products of chemically depolymerized lignin into 2-pyrone-4-6-dicarboxylic acid with: Novosphingobium aromaticivorans. Green Chem 21:1340-1350.
32. Perez J M, Kontur W S, Gehl C, Gille D M, Ma Y, Niles A V., Umana G, Donohue T J, Noguera D R. 2021. Redundancy in Aromatic O-Demethylation and Ring-Opening Reactions in Novosphingobium aromaticivorans and Their Impact in the Metabolism of Plant-Derived Phenolics. Appl Environ Microbiol 87:1-23.
33. Linz A M, Ma Y, Perez J M, Myers K S, Kontur W S, Noguera D R, Donohue T J. 2021. Aromatic Dimer Dehydrogenases from Novosphingobium aromaticivorans Reduce Monoaromatic Diketones. Appl Environ Microbiol 87.
34. Jung H M, Jung M Y, Oh M K. 2015. Metabolic engineering of Klebsiella pneumoniae for the production of cis,cis-muconic acid. Appl Microbiol Biotechnol 99:5217-5225.
35. Kasai D, Fujinami T, Abe T, Mase K, Katayama Y, Fukuda M, Masai E. 2009. Uncovering the protocatechuate 2,3-cleavage pathway genes. J Bacteriol 191:6758-6768.
36. Yamanashi T, Kim S Y, Hara H, Funa N. 2015. In vitro reconstitution of the catabolic reactions catalyzed by PcaHG, PcaB, and PcaL: The protocatechuate branch of the 0-ketoadipate pathway in Rhodococcus jostii RHA1. Biosci Biotechnol Biochem 79:830-835.
37. Johnson C W, Salvachda D, Rorrer N A, Black B A, Vardon D R, St. John P C, Cleveland N S, Dominick G, Elmore J R, Grundl N, Khanna P, Martinez C R, Michener W E, Peterson D J, Ramirez K J, Singh P, VanderWall T A, Wilson A N, Yi X, Biddy M J, Bomble Y J, Guss A M, Beckham G T. 2019. Innovative Chemicals and Materials from Bacterial Aromatic Catabolic Pathways. Joule 3:1523-1537.
38. Lupa B, Lyon D, Gibbs M D, Reeves R A, Wiegel J. 2005. Distribution of genes encoding the microbial non-oxidative reversible hydroxyarylic acid decarboxylases/phenol carboxylases. Genomics 86:342-351.
39. Payer S E, Marshall S A, Barland N, Sheng X, Reiter T, Dordic A, Steinkellner G, Wuensch C, Kaltwasser S, Fisher K, Rigby S E J, Macheroux P, Vonck J, Gruber K, Faber K, Himo F, Leys D, Pavkov-Keller T, Glueck S M. 2017. Regioselective para-Carboxylation of Catechols with a Prenylated Flavin Dependent Decarboxylase. Angew Chemie—Int Ed 56:13893-13897.
40. Marshall S A, Payne K A P, Leys D. 2017. The UbiX-UbiD system: The biosynthesis and use of prenylated flavin (prFMN). Arch Biochem Biophys. Academic Press Inc. https://doi.org/10.1016/j.abb.2017.07.014.
41. Marshall S A, Payne K A P, Fisher K, White M D, Ni Cheallaigh A, Balaikaite A, Rigby S E J, Leys D. 2019. The UbiX flavin prenyltransferase reaction mechanism resembles class I terpene cyclase chemistry. Nat Commun 10:1-10.
42. Marshall S A, Payne K A P, Fisher K, Gahloth D, Bailey S S, Balaikaite A, Saaret A, Gostimskaya I, Aleku G, Huang H, Rigby S E J, Procter D, Leys D. 2019. Heterologous production, reconstitution and EPR spectroscopic analysis of prFMN dependent enzymes, p. 489-508. In Methods in Enzymology. Academic Press Inc.
43. Payne K A P, White M D, Fisher K, Khara B, Bailey S S, Parker D, Rattray N J W, Trivedi D K, Goodacre R, Beveridge R, Barran P, Rigby S E J, Scrutton N S, Hay S, Leys D. 2015. New cofactor supports α,β-unsaturated acid decarboxylation via 1,3-dipolar cycloaddition. Nature 522:497-501.
44. Wang P H, Khusnutdinova A N, Luo F, Xiao J, Nemr K, Flick R, Brown G, Mahadevan R, Edwards E A, Yakunin A F. 2018. Biosynthesis and Activity of Prenylated FMN Cofactors. Cell Chem Biol 25:560-570.e6.
45. Payer S E, Faber K, Glueck S M. 2019. Non-Oxidative Enzymatic (De)Carboxylation of (Hetero)Aromatics and Acrylic Acid Derivatives. Adv Synth Catal 361:2402-2420.
46. Benitez F J, Beltran-heredia J, Gonzalez T, Acero J L. 1994. ACID 28:2095-2100.
47. Guo X, Wang X, Chen T, Lu Y, Zhang H. 2020. Comparing E. coli mono-cultures and co-cultures for biosynthesis of protocatechuic acid and hydroquinone. Biochem Eng J 156:107518.
48. Orn O E, Sacchetto S, van Niel E W J, Hatti-Kaul R. 2021. Enhanced Protocatechuic Acid Production From Glucose Using Pseudomonas putida 3-Dehydroshikimate Dehydratase Expressed in a Phenylalanine-Overproducing Mutant of Escherichia coli. Front Bioeng Biotechnol 9:1-10.
49. Kohlstedt M, Starck S, Barton N, Stolzenberger J, Selzer M, Mehlmann K, Schneider R, Pleissner D, Rinkel J, Dickschat J S, Venus J, B. J. H. van Duuren J, Wittmann C. 2018. From lignin to nylon: Cascaded chemical and biochemical conversion using metabolically engineered Pseudomonas putida. Metab Eng 47:279-293.
50. Guzik U, Hupert-Kocurek K, Sitnik M, Wojcieszynska D. 2013. High activity catechol 1,2-dioxygenase from Stenotrophomonas maltophilia strain KB2 as a useful tool in cis,cis-muconic acid production. Antonie van Leeuwenhoek, Int J Gen Mol Microbiol 103:1297-1307.
51. Unda F, Mottiar Y, Mahon E L, Karlen S D, Kim K H, Loqué D, Eudes A, Ralph J, Mansfield S D. 2022. A new approach to zip-lignin: 3,4-dihydroxybenzoate is compatible with lignification. New Phytol 235:234-246.
52. Umana G E, Perez J M, Unda F, Lin C-Y, Sener C, Karlen S D, Mansfield S D, Eudes A, Ralph J, Donohue T J, Noguera D R. 2022. Biological funneling of phenolics from transgenic plants engineered to express the bacterial 3-dehydroshikimate dehydratase (qsuB) gene. Front Chem Eng 4:1-12.
53. Liu H, Jin Y, Zhang R, Ning Y, Yu Y, Xu P, Deng L, Wang F. 2023. Recent advances and perspectives on production of value-added organic acids through metabolic engineering. Biotechnol Adv 62:108076.
54. Choi S, Lee H N, Park E, Lee S J, Kim E S. 2020. Recent advances in microbial production of cis,cis-muconic acid. Biomolecules 10:1-14.
55. Kamimura N, Sakamoto S, Mitsuda N, Masai E, Kajita S. 2019. Advances in microbial lignin degradation and its applications. Curr Opin Biotechnol 56:179-186.
56. Balaikaite A, Chisanga M, Fisher K, Heyes D J, Spiess R, Leys D. 2020. Ferulic Acid Decarboxylase Controls Oxidative Maturation of the Prenylated Flavin Mononucleotide Cofactor. ACS Chem Biol 15:2466-2475.
57. Becker J, Kuhl M, Kohlstedt M, Starck S, Wittmann C. 2018. Metabolic engineering of Corynebacterium glutamicum for the production of cis, cis-muconic acid from lignin. Microb Cell Fact 17:1-14.
58. Han L, Liu P, Sun J, Wu Y, Zhang Y, Chen W, Lin J, Wang Q, Ma Y. 2015. Engineering catechol 1, 2-dioxygenase by design for improving the performance of the cis, cis-muconic acid synthetic pathway in Escherichia coli OPEN https://doi.org/10.1038/srep13435.
59. A Schafer 1, A Tauch, W Jager, J Kalinowski, G Thierbach, A Piihler. 1994. Small mobilizable multi-purpose cloning vectors derived from the. Gene 145:69-73.
60. Kontur W S, Bingman C A, Olmsted C N, Wassarman D R, Ulbrich A, Gall D L, Smith R W, Yusko L M, Fox B G, Noguera D R, Coon J J, Donohue T J. 2018. Novosphingobium aromaticivorans uses a Nu-class glutathione S-transferase as a glutathione lyase in breaking the -aryl ether bond of lignin. J Biol Chem 293:4955-4968.
61. Tartoff, K. D, Hobbs C. 1987. Improved Media for Growing Plasmid and Cosmid Clones. Besthesda Res Lab Focus 9.
62. Khusnutdinova A N, Xiao J, Wang P H, Batyrova K A, Flick R, Edwards E A, Yakunin A F. 2019. Prenylated FMN: Biosynthesis, purification, and Fdcl activationMethods in Enzymology, 1st ed. Elsevier Inc. http://dx.doi.org/10.1016/bs.mie.2019.03.021.

### EMBODIMENTS

1. A recombinant microorganism comprising:
one or more modifications with respect to a corresponding microorganism not comprising the one or more modifications, wherein the one or more modifications comprise at least one of:
   a modification that increases flavin prenyltransferase activity with respect to the corresponding microorganism;
   a modification that increases protocatechuate decarboxylase activity with respect to the corresponding microorganism;
   a recombinant protocatechuate decarboxylase D gene encoding a protocatechuate decarboxylase D protein;
   a modification that increases catechol 1,2-dioxygenase activity with respect to the corresponding microorganism;
   a modification that decreases muconate lactonizing enzyme activity with respect to the corresponding microorganism;
   a modification that decreases muconolactone isomerase activity with respect to the corresponding microorganism;
   a modification that decreases catechol 2,3-dioxygenase activity with respect to the corresponding microorganism; and
   a modification that decreases protocatechuate 4,5-dioxygenase activity with respect to the corresponding microorganism.
2. The recombinant microorganism of embodiment 1, wherein the one or more modifications comprise one or more genetic modifications in the recombinant microorganism with respect to the corresponding microorganism.
3. The recombinant microorganism of any prior embodiment, wherein the one or more modifications comprise the modification that increases flavin prenyltransferase activity with respect to the corresponding microorganism.
4. The recombinant microorganism of any prior embodiment, wherein the modification that increases flavin prenyltransferase activity with respect to the corresponding microorganism, if present in the recombinant microorganism, comprises a recombinant gene encoding a flavin prenyltransferase.
5. The recombinant microorganism of any prior embodiment, wherein the modification that increases flavin prenyltransferase activity with respect to the corresponding microorganism, if present in the recombinant microorganism, comprises a recombinant gene encoding a flavin prenyltransferase comprising a sequence having at least 80%, at least 85%, at least 90%, at least 95%, or at least 99% sequence identity to SEQ ID NO:2.
6. The recombinant microorganism of any prior embodiment, wherein the one or more modifications comprise the modification that increases protocatechuate decarboxylase activity with respect to the corresponding microorganism.
7. The recombinant microorganism of any prior embodiment, wherein the modification that increases protocatechuate decarboxylase activity with respect to the corresponding microorganism, if present in the recombinant microorganism, comprises a recombinant gene encoding a protocatechuate decarboxylase.
8. The recombinant microorganism of any prior embodiment, wherein the modification that increases protocatechuate decarboxylase activity with respect to the corresponding microorganism, if present in the recombinant microorganism, comprises a recombinant gene encoding a protocatechuate decarboxylase comprising a sequence having at least 80%, at least 85%, at least 90%, at least 95%, or at least 99% sequence identity to SEQ ID NO:4.
9. The recombinant microorganism of any prior embodiment, wherein the one or more modifications comprise the recombinant protocatechuate decarboxylase D gene.
10. The recombinant microorganism of any prior embodiment, wherein the recombinant protocatechuate decarboxylase D gene, if present in the recombinant microorganism, encodes a protocatechuate decarboxylase D protein comprising a sequence having at least 80%, at least 85%, at least 90%, at least 95%, or at least 99% sequence identity to any one of SEQ ID NOS:6, 24, and 26.
11. The recombinant microorganism of any prior embodiment, wherein the recombinant protocatechuate decarboxylase D gene, if present in the recombinant microorganism, encodes a protocatechuate decarboxylase D protein comprising a sequence having at least 80%, at least 85%, at least 90%, at least 95%, or at least 99% sequence identity to SEQ ID NO:6.
12. The recombinant microorganism of any prior embodiment, wherein the one or more modifications comprise the modification that increases catechol 1,2-dioxygenase activity with respect to the corresponding microorganism.
13. The recombinant microorganism of any prior embodiment, wherein the modification that increases catechol 1,2-dioxygenase activity with respect to the corresponding microorganism, if present in the recombinant microorganism, comprises a recombinant gene encoding a catechol 1,2-dioxygenase.
14. The recombinant microorganism of any prior embodiment, wherein the modification that increases catechol 1,2-dioxygenase activity with respect to the corresponding microorganism, if present in the recombinant microorganism, comprises a recombinant gene encoding a catechol 1,2-dioxygenase comprising a sequence having at least 80%, at least 85%, at least 90%, at least 95%, or at least 99% sequence identity to SEQ ID NO:8.
15. The recombinant microorganism of any prior embodiment, wherein the one or more modifications comprise the modification that decreases muconate lactonizing enzyme activity with respect to the corresponding microorganism.
16. The recombinant microorganism of any prior embodiment, wherein the modification that decreases muconate lactonizing enzyme activity with respect to the corresponding microorganism, if present in the recombinant microorganism, comprises a mutation to a gene in the corresponding microorganism encoding a muconate lactonizing enzyme.
17. The recombinant microorganism of any prior embodiment, wherein the modification that decreases muconate lactonizing enzyme activity with respect to the corresponding microorganism, if present in the recombinant microorganism, comprises a mutation to a gene in the corresponding microorganism encoding a muconate lactonizing enzyme comprising a sequence having at least 80%, at least 85%, at least 90%, at least 95%, or at least 99% sequence identity to SEQ ID NO:10.
18. The recombinant microorganism of any prior embodiment, wherein the one or more modifications comprise the modification that decreases muconolactone isomerase activity with respect to the corresponding microorganism.
19. The recombinant microorganism of any prior embodiment, wherein the modification that decreases muconolactone isomerase activity with respect to the corresponding microorganism, if present in the recombinant microorganism, comprises a mutation to a gene in the corresponding microorganism encoding a muconolactone isomerase.
20. The recombinant microorganism of any prior embodiment, wherein the modification that decreases muconolactone isomerase activity with respect to the corresponding microorganism, if present in the recombinant microorganism, comprises a mutation to a gene in the corresponding microorganism encoding a muconolactone isomerase comprising a sequence having at least 80%, at least 85%, at least 90%, at least 95%, or at least 99% sequence identity to SEQ ID NO:12.
21. The recombinant microorganism of any prior embodiment, wherein the one or more modifications comprise the modification that decreases catechol 2,3-dioxygenase activity with respect to the corresponding microorganism.
22. The recombinant microorganism of any prior embodiment, wherein the modification that decreases catechol 2,3-dioxygenase activity with respect to the corresponding microorganism, if present in the recombinant microorganism, comprises a mutation to a gene in the corresponding microorganism encoding a catechol 2,3-dioxygenase.
23. The recombinant microorganism of any prior embodiment, wherein the modification that decreases catechol 2,3-dioxygenase activity with respect to the corresponding microorganism, if present in the recombinant microorganism, comprises a mutation to a gene in the corresponding microorganism encoding a catechol 2,3-dioxygenase comprising a sequence having at least 80%, at least 85%, at least 90%, at least 95%, or at least 99% sequence identity to SEQ ID NO:14.
24. The recombinant microorganism of any prior embodiment, wherein the one or more modifications comprise the modification that decreases protocatechuate 4,5-dioxygenase activity with respect to the corresponding microorganism.
25. The recombinant microorganism of any prior embodiment, wherein the modification that decreases protocatechuate 4,5-dioxygenase activity with respect to the corresponding microorganism, if present in the recombinant microorganism, comprises a mutation to a gene in the corresponding microorganism encoding a protocatechuate 4,5-dioxygenase subunit.
26. The recombinant microorganism of any prior embodiment, wherein the modification that decreases protocatechuate 4,5-dioxygenase activity with respect to the corresponding microorganism, if present in the recombinant microorganism, comprises a mutation to any one, two, three, or each of:
   a gene in the corresponding microorganism encoding a protocatechuate 4,5-dioxygenase subunit comprising a sequence having at least 80%, at least 85%, at least 90%, at least 95%, or at least 99% sequence identity to SEQ ID NO:16;
   a gene in the corresponding microorganism encoding a protocatechuate 4,5-dioxygenase subunit comprising a sequence having at least 80%, at least 85%, at least 90%, at least 95%, or at least 99% sequence identity to SEQ ID NO:18;
   a gene in the corresponding microorganism encoding a protocatechuate 4,5-dioxygenase subunit comprising a sequence having at least 80%, at least 85%, at least 90%, at least 95%, or at least 99% sequence identity to SEQ ID NO:20; and
   a gene in the corresponding microorganism encoding a protocatechuate 4,5-dioxygenase subunit comprising a sequence having at least 80%, at least 85%, at least 90%, at least 95%, or at least 99% sequence identity to SEQ ID NO: 22.
27. The recombinant microorganism of any prior embodiment, wherein the recombinant microorganism exhibits enhanced production of cis,cis-muconic acid with respect to the corresponding microorganism.
28. The recombinant microorganism of any prior embodiment, wherein the recombinant microorganism is a bacterium.
29. The recombinant microorganism of any prior embodiment, wherein the recombinant microorganism is a phenol-degrading microorganism.
30. The recombinant microorganism of any prior embodiment, wherein the recombinant microorganism is from the genus Novosphingobium.
31. The recombinant microorganism of any prior embodiment, wherein the recombinant microorganism is Novosphingobium aromaticivorans.
32. A method for producing cis,cis-muconic acid comprising culturing the recombinant microorganism of any prior embodiment in a medium.
33. The method of embodiment 32, wherein the medium comprises a plant-derived phenolic.
34. The method of any one of embodiments 32-33, wherein the medium comprises a plant-derived phenolic selected from the group consisting of a syringyl phenolic, a guaiacyl phenolic, and a p-hydroxyphenyl phenolic.
35. The method of any one of embodiments 32-34, wherein the medium comprises depolymerized lignin.
36. The method of any one of embodiments 32-35, further comprising isolating the cis,cis-muconic acid from the medium and/or the recombinant microorganism.

What is claimed is:

1. A recombinant microorganism comprising:
one or more modifications with respect to a corresponding microorganism not comprising the one or more modifications, wherein the one or more modifications comprise at least one of:
a modification that increases flavin prenyltransferase activity with respect to the corresponding microorganism;
a modification that increases protocatechuate decarboxylase activity with respect to the corresponding microorganism;
a recombinant protocatechuate decarboxylase D gene encoding a protocatechuate decarboxylase D protein;
a modification that increases catechol 1,2-dioxygenase activity with respect to the corresponding microorganism;
a modification that decreases muconate lactonizing enzyme activity with respect to the corresponding microorganism;
a modification that decreases muconolactone isomerase activity with respect to the corresponding microorganism;
a modification that decreases catechol 2,3-dioxygenase activity with respect to the corresponding microorganism; and
a modification that decreases protocatechuate 4,5-dioxygenase activity with respect to the corresponding microorganism.

2. The recombinant microorganism of claim 1, wherein:
the one or more modifications comprise the modification that increases flavin prenyltransferase activity with respect to the corresponding microorganism, and the modification that increases flavin prenyltransferase activity with respect to the corresponding microorganism comprises a recombinant gene encoding a flavin prenyltransferase comprising a sequence having at least 80%, at least 85%, at least 90%, at least 95%, or at least 99% sequence identity to SEQ ID NO:2; and/or
the one or more modifications comprise the modification that increases protocatechuate decarboxylase activity with respect to the corresponding microorganism, and the modification that increases protocatechuate decarboxylase activity with respect to the corresponding microorganism comprises a recombinant gene encoding a protocatechuate decarboxylase comprising a sequence having at least 80%, at least 85%, at least 90%, at least 95%, or at least 99% sequence identity to SEQ ID NO:4; and/or
the one or more modifications comprise the recombinant protocatechuate decarboxylase D gene, and the recombinant protocatechuate decarboxylase D gene encodes a protocatechuate decarboxylase D protein comprising a sequence having at least 80%, at least 85%, at least 90%, at least 95%, or at least 99% sequence identity to SEQ ID NO:6; and/or
the one or more modifications comprise the modification that increases catechol 1,2-dioxygenase activity with respect to the corresponding microorganism, and the modification that increases catechol 1,2-dioxygenase activity with respect to the corresponding microorganism comprises a recombinant gene encoding a catechol 1,2-dioxygenase comprising a sequence having at least 80%, at least 85%, at least 90%, at least 95%, or at least 99% sequence identity to SEQ ID NO:8.

3. The recombinant microorganism of claim 1, wherein:
the one or more modifications comprise the modification that increases flavin prenyltransferase activity with respect to the corresponding microorganism, and the modification that increases flavin prenyltransferase activity with respect to the corresponding microorganism comprises a recombinant gene encoding a flavin prenyltransferase comprising a sequence having at least 95% sequence identity to SEQ ID NO:2; and/or
the one or more modifications comprise the modification that increases protocatechuate decarboxylase activity with respect to the corresponding microorganism, and the modification that increases protocatechuate decarboxylase activity with respect to the corresponding microorganism comprises a recombinant gene encoding a protocatechuate decarboxylase comprising a sequence having at least 95% sequence identity to SEQ ID NO:4; and/or
the one or more modifications comprise the recombinant protocatechuate decarboxylase D gene, and the recombinant protocatechuate decarboxylase D gene encodes a protocatechuate decarboxylase D protein comprising a sequence having at least 95% sequence identity to SEQ ID NO:6; and/or
the one or more modifications comprise the modification that increases catechol 1,2-dioxygenase activity with respect to the corresponding microorganism, and the modification that increases catechol 1,2-dioxygenase activity with respect to the corresponding microorganism comprises a recombinant gene encoding a catechol 1,2-dioxygenase comprising a sequence having at least 95% sequence identity to SEQ ID NO:8.

4. The recombinant microorganism of claim 1, wherein:
the one or more modifications comprise the modification that increases protocatechuate decarboxylase activity with respect to the corresponding microorganism; and
the one or more modifications comprise the modification that increases catechol 1,2-dioxygenase activity with respect to the corresponding microorganism, and the modification that increases catechol 1,2-dioxygenase activity with respect to the corresponding microorganism comprises a recombinant gene encoding a catechol 1,2-dioxygenase comprising a sequence having at least 80%, at least 85%, at least 90%, at least 95%, or at least 99% sequence identity to SEQ ID NO:8.

5. The recombinant microorganism of claim 4, wherein the recombinant microorganism is a phenol-degrading bacterium.

6. The recombinant microorganism of claim 4, wherein the recombinant microorganism is from the genus Novosphingobium.

7. The recombinant microorganism of claim 4, wherein:
the one or more modifications comprise the modification that decreases catechol 2,3-dioxygenase activity with respect to the corresponding microorganism; and
the one or more modifications comprise the modification that decreases protocatechuate 4,5-dioxygenase activity with respect to the corresponding microorganism.

8. The recombinant microorganism of claim 7, wherein the recombinant microorganism is a phenol-degrading bacterium.

9. The recombinant microorganism of claim 7, wherein the recombinant microorganism is from the genus Novosphingobium.

10. The recombinant microorganism of claim 7, wherein:
the one or more modifications comprise the modification that increases flavin prenyltransferase activity with respect to the corresponding microorganism;
the one or more modifications comprise the recombinant protocatechuate decarboxylase D gene encoding a protocatechuate decarboxylase D protein;
the one or more modifications comprise the modification that decreases muconate lactonizing enzyme activity with respect to the corresponding microorganism; and
the one or more modifications comprise the modification that decreases muconolactone isomerase activity with respect to the corresponding microorganism.

11. The recombinant microorganism of claim 10, wherein the recombinant microorganism is a phenol-degrading bacterium.

12. The recombinant microorganism of claim 10, wherein the recombinant microorganism is from the genus Novosphingobium.

13. The recombinant microorganism of claim 10, wherein:
the modification that increases flavin prenyltransferase activity with respect to the corresponding microorganism comprises a recombinant gene encoding a flavin prenyltransferase comprising a sequence having at least 95% sequence identity to SEQ ID NO:2;
the modification that increases protocatechuate decarboxylase activity with respect to the corresponding microorganism comprises a recombinant gene encoding a protocatechuate decarboxylase comprising a sequence having at least 95% sequence identity to SEQ ID NO:4;
the recombinant protocatechuate decarboxylase D gene encodes a protocatechuate decarboxylase D protein comprising a sequence having at least 95% sequence identity to any one of SEQ ID NOS:6, 24, and 26;
the modification that increases catechol 1,2-dioxygenase activity with respect to the corresponding microorganism comprises a recombinant gene encoding a catechol 1,2-dioxygenase comprising a sequence having at least 95% sequence identity to SEQ ID NO:8;
the modification that decreases muconate lactonizing enzyme activity with respect to the corresponding microorganism comprises a mutation to a gene in the corresponding microorganism encoding a muconate lactonizing enzyme comprising a sequence having at least 95% sequence identity to SEQ ID NO:10;
the modification that decreases muconolactone isomerase activity with respect to the corresponding microorganism comprises a mutation to a gene in the corresponding microorganism encoding a muconolactone isomerase comprising a sequence having at least 95% sequence identity to SEQ ID NO:12;
the modification that decreases catechol 2,3-dioxygenase activity with respect to the corresponding microorganism comprises a mutation to a gene in the corresponding microorganism encoding a catechol 2,3-dioxygenase comprising a sequence having at least 95% sequence identity to SEQ ID NO:14; and
the modification that decreases protocatechuate 4,5-dioxygenase activity with respect to the corresponding microorganism comprises a mutation to any one, two, three, or each of:
a gene in the corresponding microorganism encoding a protocatechuate 4,5-dioxygenase subunit comprising a sequence having at least 95% sequence identity to SEQ ID NO:16;
a gene in the corresponding microorganism encoding a protocatechuate 4,5-dioxygenase subunit comprising a sequence having at least 95% sequence identity to SEQ ID NO:18;
a gene in the corresponding microorganism encoding a protocatechuate 4,5-dioxygenase subunit comprising a sequence having at least 95% sequence identity to SEQ ID NO:20; and
a gene in the corresponding microorganism encoding a protocatechuate 4,5-dioxygenase subunit comprising a sequence having at least 95% sequence identity to SEQ ID NO: 22.

14. The recombinant microorganism of claim 13, wherein the recombinant microorganism is a phenol-degrading bacterium.

15. The recombinant microorganism of claim 13, wherein the recombinant microorganism is from the genus Novosphingobium.

16. The recombinant microorganism of claim 13, wherein the recombinant microorganism exhibits enhanced production of cis,cis-muconic acid with respect to the corresponding microorganism.

17. A method for producing cis,cis-muconic acid comprising culturing the recombinant microorganism of claim 1 in a medium comprising a plant-derived phenolic.

18. The method of claim 17, wherein the medium comprises a plant-derived phenolic selected from the group consisting of a syringyl phenolic, a guaiacyl phenolic, and a p-hydroxyphenyl phenolic.

19. The method of claim 17, wherein the medium comprises depolymerized lignin.

20. The method of claim 17, further comprising isolating the cis,cis-muconic acid from the medium and/or the recombinant microorganism.


SEQUENCE LISTING (69 sequences)

SEQ ID NO: 1 - DNA, 615 bp, Novosphingobium aromaticivorans
  source 1..615: mol_type = genomic DNA
gtgaagcgcatggtcgtggggattaccggcgcaaccggctcggtctatggtcttcgcctgcttgagctgctgcgcgagacgggcggttgggaaacccatctggtaatgtctccggctgcgctgctcaacattcgcgaggaactgcccgaaggcaaagcccggctcgaagcgctggccgatgtggtgcacaacgtccgcaacgtcggcgcctcgatcgccagcggttcgttcgtatgcgaaggcatggcgattgcgccctgttcgatgcgcacgctgggcgcggtggcgcacgccctgtccgacaaccttatcacccgcgcggccgacgtgatgctgaaggaacggcgccgcctggtgatgatcacccgcgaagcgccgctcaacctggcgcacctgcgcaacatgacggcctgcaccgaaatgggggcggtgatcttccccccggtgccggccttctatgcgcggccgacctcgctggccgacgtggtcgatcacacctgcatgcgggtactggatctgttcgggcttcatgcgaagtcggagaaacgctggcaaggccttagcaaagaggcggcaagccttgttccgggtgctgggcaaatggaagggaattga

SEQ ID NO: 2 - protein, 204 aa, Novosphingobium aromaticivorans
MKRMVVGITGATGSVYGLRLLELLRETGGWETHLVMSPAALLNIREELPEGKARLEALADVVHNVRNVGASIASGSFVCEGMAIAPCSMRTLGAVAHALSDNLITRAADVMLKERRRLVMITREAPLNLAHLRNMTACTEMGAVIFPPVPAFYARPTSLADVVDHTCMRVLDLFGLHAKSEKRWQGLSKEAASLVPGAGQMEGN

SEQ ID NO: 3 - DNA, 1461 bp, Novosphingobium aromaticivorans
  source 1..1461: mol_type = genomic DNA
atgaccatgaacgatctccctaaccgcgcccgctcgatctcgtcgctgcgcgacttcctcgaactgctcgaggatgccggccaggcgatcacctggagcgatgcggtgatgcccgaacccggcgtgcgcaacatagccgtcgccgcatcgcgcgatgccaacggcgcgccggcgatcgtattcgacaatatcaccggttaccccggcaagcgcttggcggtgggcgtccatggttcgtgggacaacatcgccctgctgctgggccgacctaaaggcacgaccatccgcgagcttttcttcgagatcgccggccgctggggcgatcaggaagcgcaaatcagctttgtcccagaagcccaggccccggtgcacgaatgccggatcgaacaggacatcaacctttacgatgtcctgccggtctatcggatcaacgaatacgatggcgggttctacatcggcaaggcctcggtcgcctcgcgcgatccgctcgatccagacaatttcggcaagcagaatgtcggcatctatcgcctgcagatccaggggccggacaccttcaccctgatgacgatcccctcccacgacatgggacgtcagatcatggcggccgaacgggaaggcgttccgctaaagattgcggtcatgctgggtaatcatcccggccttgcggtgtttgctgccaccccgatcggctacgaggaatcggaatattcctatgcctcggcgatgatgggcgcgccaatccggctgaccaaatcgggcaacgggatcgacatcctggccgacagcgaaatcgtgatagaggccgaactgcaaccgggtggacgcgagctggaagggccgttcggcgaattccccggttcctacagcggcgtgcgcaaggcgccgatcttcaaggtcacggcggtgtcgcaccggcgcgatccgatcttcgagaacatttacatcgggcgcggctggaccgagcacgatacgctgatcggcctgcacacctccgccccgatctatgcccagctgcgccagagcttccccgaagtcaccgcggtcaacgcgctttaccagcacggactgaccgggatcatctcggtcaaaaaccgcatggccggctttgccaagacggtcgcgctgcgcgcgctgagcacgccgcacggcgtgatgtacctcaagaacctgattatggtcgatgccgatgtcgatccgttcgatctcaaccaagtgatgtgggcgctttcgacccgcacccgtgcggacgatatcatcgtgctgcccaacatgcctgccgtgccgatcgatccttcggcagtggtcccgggcaaggggcaccgcctgatcatcgacgcgaccagctatctcccgcccgatccggtgggtgaagcgcaccttgtcaccccgccgaccggggacgagatcgacgccctgagcaagcggatccgcgaaatgcagctgggagccctgtcatga

SEQ ID NO: 4 - protein, 486 aa, Novosphingobium aromaticivorans
MTMNDLPNRARSISSLRDFLELLEDAGQAITWSDAVMPEPGVRNIAVAASRDANGAPAIVFDNITGYPGKRLAVGVHGSWDNIALLLGRPKGTTIRELFFEIAGRWGDQEAQISFVPEAQAPVHECRIEQDINLYDVLPVYRINEYDGGFYIGKASVASRDPLDPDNFGKQNVGIYRLQIQGPDTFTLMTIPSHDMGRQIMAAEREGVPLKIAVMLGNHPGLAVFAATPIGYEESEYSYASAMMGAPIRLTKSGNGIDILADSEIVIEAELQPGGRELEGPFGEFPGSYSGVRKAPIFKVTAVSHRRDPIFENIYIGRGWTEHDTLIGLHTSAPIYAQLRQSFPEVTAVNALYQHGLTGIISVKNRMAGFAKTVALRALSTPHGVMYLKNLIMVDADVDPFDLNQVMWALSTRTRADDIIVLPNMPAVPIDPSAVVPGKGHRLIIDATSYLPPDPVGEAHLVTPPTGDEIDALSKRIREMQLGALS

SEQ ID NO: 5 - DNA, 225 bp, Novosphingobium aromaticivorans
  source 1..225: mol_type = genomic DNA
atgaccaccaccgtctgcgggcgctgcaaatcgagcggcgctgtcaccgatcatcagggcaggcaggacggcgcggtcgtgtggacgatcctgcgctgcccgacctgcaacttttcctggcgcgacagcgaaccggcccgcgctatcgacccggctgtgcgctcggccgatttcgccgtcgatgtcggcgatctccagcgttatcccaagattctccagcaataa

SEQ ID NO: 6 - protein, 74 aa, Novosphingobium aromaticivorans
MTTTVCGRCKSSGAVTDHQGRQDGAVVWTILRCPTCNFSWRDSEPARAIDPAVRSADFAVDVGDLQRYPKILQQ

SEQ ID NO: 7 - DNA, 897 bp, Novosphingobium aromaticivorans
  source 1..897: mol_type = genomic DNA
atgcctgccaccttcgccagttccgattccgtgcagaagctcttcgatcgcgcctgcggtcttgattgcgcaggcggcaatccccgcctcaaggcgatcatgcgcgaccttctccaggcaacggccgacatcatcgtcaagcatgacgtgtccgaaagcgagttctggcaggcgacccgctatcttgccgatggcgccggcgagatcggcctgatcgtccccggcatcggcctcgaacacttcctcgatctctacatggacgccaaggacgccgaagccggcctcaccggcggaaccccgcgcacgatcgaaggcccgctctacgtcgctggtgcaccgctggtggatggcagtgacgaagtggacctgacttccgaccccgacgataccgacacgctgcacatgaccggcacgatcaccggccccgatggcgagccggtcaaggacgcgatcctccacgtctggcacgcgaacagcaagggctggtattcgcacttcgatcccacgagcgagcagaccccgttcaacaaccgccgccgcatccgcgtccccgccgacggtcgctacgccttccgctccaagatgccgcatggctattccgtgccgccgggtggcgccaccgacgtgctgatgcaggcgctcggccgccacggcaatcgcccagcgcacgtccacttcttcgtcgaggcgccgggctaccgcacgctgaccacgcagatcaacttcggcgacgaccccttcgcggccgacgatttcgccttcggcacgcgagagggcttgctgccggtgccgagccgccagggcgataccgcccacatcgcgttcgacttccagctccagcgcgcccgctcggaggacgagcagcggttctcggaacgcacccgcgcccaggcctga

SEQ ID NO: 8 - protein, 298 aa, Novosphingobium aromaticivorans
MPATFASSDSVQKLFDRACGLDCAGGNPRLKAIMRDLLQATADIIVKHDVSESEFWQATRYLADGAGEIGLIVPGIGLEHFLDLYMDAKDAEAGLTGGTPRTIEGPLYVAGAPLVDGSDEVDLTSDPDDTDTLHMTGTITGPDGEPVKDAILHVWHANSKGWYSHFDPTSEQTPFNNRRRIRVPADGRYAFRSKMPHGYSVPPGGATDVLMQALGRHGNRPAHVHFFVEAPGYRTLTTQINFGDDPFAADDFAFGTREGLLPVPSRQGDTAHIAFDFQLQRARSEDEQRFSERTRAQA

SEQ ID NO: 9 - DNA, 1164 bp, Novosphingobium aromaticivorans
  source 1..1164: mol_type = genomic DNA
atgaccgcgctcgccaacccccagattctcggcatcgagacgattcttctcgatctgccgaccatccgtccgcacgtgctggccatggccacgatgcacgcccagacgatctgcctggttcgcctgacctgctccgatggtatcgtcggattgggcgaggcgaccacgatcggcggcctcgcatatggcccggaagccccggaaacgatcaagaccgccatcgacacctacttcgccccgcttcttgccgggcaggatgccacgcgccccgccgcggccatggcgctcgtcgcccgccacgtcgtcgggaatcacttcgccaagtgcgcgatcgagaccgcgctgctcgacgcacagggcaagcgacttggccttccggtcagtgaactccttggcggccgccgcgtggattcgctaccggtgctctggacgctcgccagcggcgataccgcgcgcgacatcgcagaggcggagcagatgctcgacacgcgccgacacgacgcgttcaagctcaagatcggcaagcgcccgatcgaacaggatgtcgcccatgtcggcgcgatcaaggctgcgctcggcgaccgggcttcggtccgcgtcgacgtcaacatggcgtgggacgaacccacggcgcggcgcggtcttgccatgctggccgatgcgggatgcgacctcgtcgaacaaccgatcatccgccacaaccgcgatggcatggcccgcctcgtcgcgctggggctggtcccggtcatggctgacgagagccttaccggtccggccagcgcgatggacttcgcccgcgccgccgccgccgatgtcttcgccgtgaagatcgagcagtccggcgggctcgatgcggcgcgcgcggtggcgcagatcggcgatgcggcctgcatcggcctttatggcggcaccatgctcgaaggcgccatcggtaccatcgcatcggcccacgcttttgccactttcccggcgttgaagtggggcaccgaacttttcggcccgctcctcctcacggaggaaatcctcgagcgccccctcacctacgccgatttctcgctcgaagtgccggcggggcccggtctcggcatcgccctcgacgaagaccgcgtcgaacacttccggcgcgatcgcaccgcaacccagttcgccttgcaaggagcctga

SEQ ID NO: 10 - protein, 387 aa, Novosphingobium aromaticivorans
MTALANPQILGIETILLDLPTIRPHVLAMATMHAQTICLVRLTCSDGIVGLGEATTIGGLAYGPEAPETIKTAIDTYFAPLLAGQDATRPAAAMALVARHVVGNHFAKCAIETALLDAQGKRLGLPVSELLGGRRVDSLPVLWTLASGDTARDIAEAEQMLDTRRHDAFKLKIGKRPIEQDVAHVGAIKAALGDRASVRVDVNMAWDEPTARRGLAMLADAGCDLVEQPIIRHNRDGMARLVALGLVPVMADESLTGPASAMDFARAAAADVFAVKIEQSGGLDAARAVAQIGDAACIGLYGGTMLEGAIGTIASAHAFATFPALKWGTELFGPLLLTEEILERPLTYADFSLEVPAGPGLGIALDEDRVEHFRRDRTATQFALQGA

SEQ ID NO: 11 - DNA, 291 bp, Novosphingobium aromaticivorans
  source 1..291: mol_type = genomic DNA
atgctgttccaggtcgaaatgaccgtcgatatcccgctggatttcgacaaggcagaggccgaacggatcaaggccgtcgaaaaggcctattcccacgaacttcagcgcagcggcgaatggcgccacatctggcgggtcgccggacaatacgccaacctgtcgatcttcgatgtggcaagcaacgagcgcctgcacgagatcctcatgggactgccgctctatcccttcatgaccatcaaggtcacgccgctttgccggcacccctcttccattcgcgacgacgatcgctga

SEQ ID NO: 12 - protein, 96 aa, Novosphingobium aromaticivorans
MLFQVEMTVDIPLDFDKAEAERIKAVEKAYSHELQRSGEWRHIWRVAGQYANLSIFDVASNERLHEILMGLPLYPFMTIKVTPLCRHPSSIRDDDR

SEQ ID NO: 13 - DNA, 924 bp, Novosphingobium aromaticivorans
  source 1..924: mol_type = genomic DNA
atggctctaactggtgtaattcgtcctggctacgtgcagctcagggttctcgacctcgatgaggctattggccattaccgcgaccggattggtttgaatttcgtcaatcgcgacggcgaccgcgcctttttccaggcgtttgacgaattcgatcggcacagcattatcctgcgcgaggccgaccaagccgggatggacgtgatgggctttaaggtcgtcaaggatgccgacctcgaccatttcgcggagcgcctgctcgatatcggcgtccatgtcgatgtgatcccggccggatcggaccccggtgtgggtcgcaagatccgcttcaacacgccaacccagcacgtgttcgaactctacgccgaaatggaactgtcggccaccggcccggcggtcaagaaccccgatgtctgggtggtcgaaccgcgcggcatgcgtgccacccgattcgatcactgcgcactgaatggcgtggacatctccgcatccgcaaaaatcttcgtcgatgcgctggatttctcggttaccgaggagctggtcgatgaaggcagtgggacgcgcctcggcatcttcctgagctgcagcaacaaggcgcatgatgttgccttcctcggttatcccgaagacggcaagatccaccacacctctttcaacctcgaatcctggcacgacgtcggccatgcagccgacatcatcagccgctacgatatttcgctcgatatcgggccgacgcgtcacgggatcacccgcggccagacgatctacttcttcgatccctcgggtaaccgcaacgaaaccttcagcggcggttacatctattatcccgacaatccgcagcgcatgtggcaggcagagagcgccggcaaggcgatcttctactacgaaaaggcgctgaacgaccgcttcatgacggtgaacacctga

SEQ ID NO: 14 - protein, 307 aa, Novosphingobium aromaticivorans
MALTGVIRPGYVQLRVLDLDEAIGHYRDRIGLNFVNRDGDRAFFQAFDEFDRHSIILREADQAGMDVMGFKVVKDADLDHFAERLLDIGVHVDVIPAGSDPGVGRKIRFNTPTQHVFELYAEMELSATGPAVKNPDVWVVEPRGMRATRFDHCALNGVDISASAKIFVDALDFSVTEELVDEGSGTRLGIFLSCSNKAHDVAFLGYPEDGKIHHTSFNLESWHDVGHAADIISRYDISLDIGPTRHGITRGQTIYFFDPSGNRNETFSGGYIYYPDNPQRMWQAESAGKAIFYYEKALNDRFMTVNT

SEQ ID NO: 15 - DNA, 438 bp, Novosphingobium aromaticivorans
  source 1..438: mol_type = genomic DNA
gtgactgacaacagctcgaccgataagcccaagcctgtgcagaacatccacgagtacctcgccgagttcgaggacatccccggcacgcgcatctataccgctgcccgcgcccgcaaggggtactggatcaaccagttcgcgatgagcctgatgaagccggagaaccgcgagcggttcaaggcgaacgagcgggcctatcttgacgaatggaagatcagcgaggaggccaaggaagcgctgctcgcgcgggactacaaccgcctgctcgaccttggcggcaacgtctatttcctgtcgaagctgttctcctcggacggactgccgttcgccgaggcggtcagcacgatgaccgacatgacctggccggaataccgccagatgatgctggacggcggacgcaagcccgaaggcaaccgatcgatcaagggaggctattga

SEQ ID NO: 16 - protein, 145 aa, Novosphingobium aromaticivorans
MTDNSSTDKPKPVQNIHEYLAEFEDIPGTRIYTAARARKGYWINQFAMSLMKPENRERFKANERAYLDEWKISEEAKEALLARDYNRLLDLGGNVYFLSKLFSSDGLPFAEAVSTMTDMTWPEYRQMMLDGGRKPEGNRSIKGGY

SEQ ID NO: 17 - DNA, 843 bp, Novosphingobium aromaticivorans
  source 1..843: mol_type = genomic DNA
atggcccgcataaccgctggcgttggctccagccacgttccgctgctgggtgtcgctgttgatcagggcaagtggcaggacgactatttcggcccgatcttcaagggttatgaatggacccgcgaatgggaaaagcgcgagaagcccgatgtggtcattctggtctacaacgaccacgcttcggcattcgacgcgaacatcatcccgaccttcgcaatcggctgcggcgagcactacaagtcggccgatgaaggctggggcccgcgcccggtgcccgacgtggaaggccatgccgaccttgcctggcacatcgcgcagagcctgatcctcgacgatttcgacatgaccatcatcaacgagatggatgtggaccacggcctgaccgtgccgctctcgatgatgttcggccagcccgagaagtggccgtgcaaggtcgtgccgctggcggtgaacgtcgtcacctatccggtgccgtcgggcaaccgctgctgggcgctgggcgaggcgatcgcccgcgcggtggaaagcttccccgaggacctcaacgtgcagatctggggcacgggcggcatgagccaccagctccagggcccgcgcgccggcctgctcaaccgcgagtgggacaacaagttcctcgacatgctggaatcggacaacgacgatgtccgctacattccgcatatcgaatacctgcgcgagaccggctcggaaggcatcgagatggtcatgtggctgatcatgcgcggcgcgctcggcaagaaggtcaagcgcctgaaccgccattaccacattccctgcagcaacaccgcgatcgggcacatcgtgctcgagcccgcggactga

SEQ ID NO: 18 - protein, 280 aa, Novosphingobium aromaticivorans
MARITAGVGSSHVPLLGVAVDQGKWQDDYFGPIFKGYEWTREWEKREKPDVVILVYNDHASAFDANIIPTFAIGCGEHYKSADEGWGPRPVPDVEGHADLAWHIAQSLILDDFDMTIINEMDVDHGLTVPLSMMFGQPEKWPCKVVPLAVNVVTYPVPSGNRCWALGEAIARAVESFPEDLNVQIWGTGGMSHQLQGPRAGLLNREWDNKFLDMLESDNDDVRYIPHIEYLRETGSEGIEMVMWLIMRGALGKKVKRLNRHYHIPCSNTAIGHIVLEPAD

SEQ ID NO: 19 - DNA, 402 bp, Novosphingobium aromaticivorans
  source 1..402: mol_type = genomic DNA
atgacacctgaaggaaaccgcgaggaccgggcggccgtggacaaggcgctccgccgcgcgattccgctgttcgatggcgatctcgccacgcgcggatacgagctcaacgcgatgtgtttttccttcaacgaaaaagccaatcgcgaggcctttctggccgatgaagaagcctattgccgcaagttcaacctgacgccgcagcaacgcaaggccgtggccgatcgcgatgtgctcgcgatgctcgatgcgggcgggaacgtctattatctggccaagctggccggcattttcggccttggcgtgcaggacctgggcgcattgcagaccggcatgtcggtcgctgatttcaaggccatgctcgtgcgctgggccgacagtattcccaacaaggagaacgcgtga

SEQ ID NO: 20 - protein, 133 aa, Novosphingobium aromaticivorans
MTPEGNREDRAAVDKALRRAIPLFDGDLATRGYELNAMCFSFNEKANREAFLADEEAYCRKFNLTPQQRKAVADRDVLAMLDAGGNVYYLAKLAGIFGLGVQDLGALQTGMSVADFKAMLVRWADSIPNKENA

SEQ ID NO: 21 - DNA, 843 bp, Novosphingobium aromaticivorans
  source 1..843: mol_type = genomic DNA
atggccaaggtcattggcggttatttcaccagccacgtcccgggcattggcggcgccatcgttcgcggcgatcaggaaacgccttattggaagccgttcttcgatggctacccgcccatccgcgaatggctggtggaggcccggcctgacgtcgcaatcgtcttttccaacgaccacggcctcaacttcttcctcgacaagatgccgacctttgccgtcggtgcggcagagcgctacgacaatgccgacgagggctggggcctgccggtctacaagagcttcgccggtcacccggcgctttcctggcacctgatcgacagtctggtgcgtgacgagttcgacatcacaacctgccagaagatgctggtcgatcacgcggtttcgatcccgttcgaactgatctacccgggtgcggagagctggccgatcaagctcgtcccgatctcgatcaacaccgtgcaatatccgctgccgagtcctaagcgctgccttgcgcttggccgtgcggtaggtcgcgcgctgcaatcctgggccggtgacgaacgtgtcctgatttgtggtaccggcgggctttcgcatcagctggacggtccacgcgcgggtttcatgaacccggactacgacatgttctgccttgataatcttgcggccaatcccgacgccctgaccggccataccgccgagcaggtagccgagcttgccggaacgcagggcgtcgagattctcaactggatcgcggcgcgcggggcaatgggcgatgtgccgctgcacgaggtcagccggaactaccatatccccatcagcaatactgcggccgccagcctcctcctcgagcctgcctga

SEQ ID NO: 22 - protein, 280 aa, Novosphingobium aromaticivorans
MAKVIGGYFTSHVPGIGGAIVRGDQETPYWKPFFDGYPPIREWLVEARPDVAIVFSNDHGLNFFLDKMPTFAVGAAERYDNADEGWGLPVYKSFAGHPALSWHLIDSLVRDEFDITTCQKMLVDHAVSIPFELIYPGAESWPIKLVPISINTVQYPLPSPKRCLALGRAVGRALQSWAGDERVLICGTGGLSHQLDGPRAGFMNPDYDMFCLDNLAANPDALTGHTAEQVAELAGTQGVEILNWIAARGAMGDVPLHEVSRNYHIPISNTAAASLLLEPA

SEQ ID NO: 23 - DNA, 222 bp, Enterobacter cloacae
  source 1..222: mol_type = genomic DNA
atgatctgcccgcgctgcgccgacgagcagatcgaggtcatggccaccagcccggtgaagggcatctggaccgtctaccagtgccagcactgcctgtacacctggcgggacaccgaaccgcttcgtcgcacctcgcgcgagcactatcccgaagcgttccgcatgacgcagaaggacatcgatgaagcgccgcaggtgcccacgattcctccgctcctgtga

SEQ ID NO: 24 - protein, 73 aa, Enterobacter cloacae
MICPRCADEQIEVMATSPVKGIWTVYQCQHCLYTWRDTEPLRRTSREHYPEAFRMTQKDIDEAPQVPTIPPLL

SEQ ID NO: 25 - DNA, 222 bp, Klebsiella pneumoniae
  source 1..222: mol_type = genomic DNA
atgatttgtccacgttgtgccgatgagcaaattgaggtgatggccacatcaccggtgaaagggatctggacggtttatcagtgccagcattgtctgtatacctggcgcgataccgagccgctgcgtcgtaccagtcgcgaacattaccctgaagcgttccgcatgacgcagaaggatattgatgaggcgccgcaggtaccgaccattcctccattgctgtaa

SEQ ID NO: 26 - protein, 73 aa, Klebsiella pneumoniae; identical to SEQ ID NO: 24

SEQ ID NO: 27 - DNA, 927 bp, Escherichia coli
  source 1..927: mol_type = genomic DNA
atgtcgaagaacccgtcgcagcagtcggaactcgaaaccctcctcgccatctcgtcgggcctcaacaccgacggcggcaacgaacgcgtcaagcgcgtcatccaccagctcctcaacgacctctgccacaccatcaagaccttcgacatctcggacgaagaattctggatcgccgtcaactacctcaacgaactcggcgaacgcaaggaagccgccctcctcgccgccggcctcggcctcgaacactacctcgacatgcgcgccgacgaaaaggaagccgcctcggtctcgcgcgccggcaccccgcgcaccatcgaaggcccgctctacgtcgccaacgccccgctctcggaccacttcgcccgcatggacgacggctcggaacgcgccgaagccatgtggctccacggcaccgtcaccgacatcgacggcaagccggtcgccggcgccatcgtcgacatctggcacgccaacacccacggcggctactcgttcttcgacccgtcgcagtcggaatacaacctccgccgccgcgtcaagaccgccgccgacggctcgtacgccgtccgctcgatcgtcccgtgcggctacggctgcccgccggacggcccgacccagaagctcctcaacgaactcggccgccacggcaaccgcccggcccacatccacttcttcgtctcggccccgggcttcaagcacctcaccacccagatcaacctcaacggcgaccgctacctctgggacgacttcgccttcgccacccgcgaagaactcatcgccgacccggtcaaggtcaccgactcgaccctcgcccgcgaacgcgacatccacgaaccgcacaccgaagtctcgttctcgttcaccctcgtcaaggccgccggcgccgaagaagaagcccgcggcaagcgcgcccgcgtcaaggaatga

SEQ ID NO: 28 - DNA, 2299 bp, Novosphingobium aromaticivorans
  source 1..2299: mol_type = genomic DNA
gtgaagcgcatggtcgtggggattaccggcgcaaccggctcggtctatggtcttcgcctgcttgagctgctgcgcgagacgggcggttgggaaacccatctggtaatgtctccggctgcgctgctcaacattcgcgaggaactgcccgaaggcaaagcccggctcgaagcgctggccgatgtggtgcacaacgtccgcaacgtcggcgcctcgatcgccagcggttcgttcgtatgcgaaggcatggcgattgcgccctgttcgatgcgcacgctgggcgcggtggcgcacgccctgtccgacaaccttatcacccgcgcggccgacgtgatgctgaaggaacggcgccgcctggtgatgatcacccgcgaagcgccgctcaacctggcgcacctgcgcaacatgacggcctgcaccgaaatgggggcggtgatcttccccccggtgccggccttctatgcgcggccgacctcgctggccgacgtggtcgatcacacctgcatgcgggtactggatctgttcgggcttcatgcgaagtcggagaaacgctggcaaggccttagcaaagaggcggcaagccttgttccgggtgctgggcaaatggaagggaattgagaatgaccatgaacgatctccctaaccgcgcccgctcgatctcgtcgctgcgcgacttcctcgaactgctcgaggatgccggccaggcgatcacctggagcgatgcggtgatgcccgaacccggcgtgcgcaacatagccgtcgccgcatcgcgcgatgccaacggcgcgccggcgatcgtattcgacaatatcaccggttaccccggcaagcgcttggcggtgggcgtccatggttcgtgggacaacatcgccctgctgctgggccgacctaaaggcacgaccatccgcgagcttttcttcgagatcgccggccgctggggcgatcaggaagcgcaaatcagctttgtcccagaagcccaggccccggtgcacgaatgccggatcgaacaggacatcaacctttacgatgtcctgccggtctatcggatcaacgaatacgatggcgggttctacatcggcaaggcctcggtcgcctcgcgcgatccgctcgatccagacaatttcggcaagcagaatgtcggcatctatcgcctgcagatccaggggccggacaccttcaccctgatgacgatcccctcccacgacatgggacgtcagatcatggcggccgaacgggaaggcgttccgctaaagattgcggtcatgctgggtaatcatcccggccttgcggtgtttgctgccaccccgatcggctacgaggaatcggaatattcctatgcctcggcgatgatgggcgcgccaatccggctgaccaaatcgggcaacgggatcgacatcctggccgacagcgaaatcgtgatagaggccgaactgcaaccgggtggacgcgagctggaagggccgttcggcgaattccccggttcctacagcggcgtgcgcaaggcgccgatcttcaaggtcacggcggtgtcgcaccggcgcgatccgatcttcgagaacatttacatcgggcgcggctggaccgagcacgatacgctgatcggcctgcacacctccgccccgatctatgcccagctgcgccagagcttccccgaagtcaccgcggtcaacgcgctttaccagcacggactgaccgggatcatctcggtcaaaaaccgcatggccggctttgccaagacggtcgcgctgcgcgcgctgagcacgccgcacggcgtgatgtacctcaagaacctgattatggtcgatgccgatgtcgatccgttcgatctcaaccaagtgatgtgggcgctttcgacccgcacccgtgcggacgatatcatcgtgctgcccaacatgcctgccgtgccgatcgatccttcggcagtggtcccgggcaaggggcaccgcctgatcatcgacgcgaccagctatctcccgcccgatccggtgggtgaagcgcaccttgtcaccccgccgaccggggacgagatcgacgccctgagcaagcggatccgcgaaatgcagctgggagccctgtcatgaccaccaccgtctgcgggcgctgcaaatcgagcggcgctgtcaccgatcatcagggcaggcaggacggcgcggtcgtgtggacgatcctgcgctgcccgacctgcaacttttcctggcgcgacagcgaaccggcccgcgctatcgacccggctgtgcgctcggccgatttcgccgtcgatgtcggcgatctccagcgttatcccaagattctccagcaataa

SEQ ID NO: 29 - DNA, 2381 bp, Escherichia coli
  source 1..2381: mol_type = genomic DNA
atgaggctcatcgtgggcatgacgggagccacgggcgctccgcttggcgtggccctcctgcaggcgctccgcgacatgcccgaggttgaaacccatctggtgatgtcgaagtgggcgaagaccaccatcgagctggaaacgccgtataccgcgcaggacgtcgctgccctggccgacgtcgtccacagccctgccgatcaggcagccaccatctcgtcgggctcgttccgcaccgatggcatgatcgtcattccctgcagcatgaagacgcttgcaggcattcgcgcgggctatgccgaagggcttgtcggtcgtgcggcagatgttgtgctgaaagaaggtcgcaagctggtgctggtcccgcgcgaaacgccgctcagcaccatccatctggagaacatgctcgcgctttcccgcatgggggtggcgatggtgccgcccatgcccgcgtactacaaccatccgcaaaccgccgacgacatcacccagcacatcgtgacccgcgtcctcgaccagttcggtctggagcacaagaaggcacgtcgctggaatggcctgcaggcggcgaagcacttcagccaggagaacaacgacggcatctgatgctgggcaaatggaagggaattgagaatgcagaaccccatcaacgacctccgctctgccatcgcgctgctgcaacgccatcccggtcactatatcgaaaccgaccacccggtcgatcccaatgctgaactggcgggcgtctatcgccatatcggcgcgggcggtaccgtcaaacgccccacccgcacgggcccggccatgatgttcaacagcgtgaagggctaccctggctcccgcatcctggtcggtatgcatgccagccgggaacgcgcggcgcttctgctgggctgtgtcccctcgaagctggcacagcacgtcggtcaggcggtgaagaacccggttgcaccggtggtggttccagcctcgcaggcaccgtgccaggagcaggtcttctatgccgacgatccggacttcgacctgcgtaagctgcttccggccccgaccaacacgccgattgatgcaggcccgttcttctgcctggggctggtcctggcaagcgatccggaagacacctcgctgaccgatgtgaccattcaccgtctctgcgtgcaggagcgagacgaactctcgatgttccttgccgccggccgccatatcgaagtctttcgcaagaaggccgaagcggcgggcaaaccgctgccggtcaccatcaacatgggacttgacccggctatctacataggggcctgcttcgaagcgcccaccacgcccttcggttacaacgagcttggcgttgccggggcactccgccagcaaccggtggagctggtccagggcgtagcggtcaaggagaaagcgatcgcgcgggcggaaatcatcatcgagggcgaactgcttcccggcgtgcgcgtccgcgaagatcagcacaccaacaccggccacgccatgccggagttcccgggctactgcggcgaggcgaatccgtcgctgccggtgatcaaggtgaaagccgtgacgatgcgaaaccatgcgatcctgcagacgctggtgggccctggcgaagagcacaccacgcttgccggtctgccgaccgaggccagcattcgcaacgcggtcgaagaggccattcccggctttctgcagaacgtctacgcccacaccgccggaggcggtaagttcctcggcatcctacaggtgaagaagcgccagccgtcggacgaaggacgtcagggccaggcggcactcatcgccctggccacctattccgagctgaagaacatcatcctcgtggacgaagacgtggacatcttcgacagcgacgacatcctgtgggcaatgaccacccgcatgcagggcgatgtgagcatcaccacgcttccggggatccgcggccaccagctggatccgtcgcagtcgccggactacagcacctcgatccgtggaaacggcatctcctgcaagactatcttcgactgcacggtgccgtgggcgctgaaggcgcggttcgaacgggcgccgttcatggaggtcgaccccacaccgtgggcgccggagctgttcagcgacaagaagtgacagctgggagccctgtcatgatctgcccgcgctgcgccgacgagcagatcgaggtcatggccaccagcccggtgaagggcatctggaccgtctaccagtgccagcactgcctgtacacctggcgggacaccgaaccgcttcgtcgcacctcgcgcgagcactatcccgaagcgttccgcatgacgcagaaggacatcgatgaagcgccgcaggtgcccacgattcctccgctcctgtgagctgaccagacaggagtagtaccc

SEQ ID NO: 30 - DNA, 56 bp, synthetic construct
  source 1..56: mol_type = other DNA
cgattcattaatgcagctggcacgacagcgatctgctcgctaaattgtggaaggag

SEQ ID NO: 31 - DNA, 41 bp, synthetic construct
  source 1..41: mol_type = other DNA
gctggagaatcttgggataaccgttcccgcctgtcagtgtc

SEQ ID NO: 32 - DNA, 47 bp, synthetic construct
  source 1..47: mol_type = other DNA
ctgacaggcgggaacggttatcccaagattctccagcaataaggctc

SEQ ID NO: 33 - DNA, 55 bp, synthetic construct
  source 1..55: mol_type = other DNA
gtttctgcggactggctttctagatgttccgtaaaggttccagtagcctagtccg

SEQ ID NO: 34 - DNA, 57 bp, synthetic construct
  source 1..57: mol_type = other DNA
cgattcattaatgcagctggcacgacaggatgttacgaagttcaccttcaccggttc

SEQ ID NO: 35 - DNA, 45 bp, synthetic construct
  source 1..45: mol_type = other DNA
ctggagaatcttgggataaccaggagactttcctgcgcgttttgg

SEQ ID NO: 36 - DNA, 57 bp, synthetic construct
  source 1..57: mol_type = other DNA
cccaaaacgcgcaggaaagtctcctggttatcccaagattctccagcaataaggctc

SEQ ID NO: 37 - DNA, 55 bp, synthetic construct; identical to SEQ ID NO: 33
  source 1..55: mol_type = other DNA

SEQ ID NO: 38 - DNA, 26 bp, synthetic construct
  source 1..26: mol_type = other DNA
ctgaccagacaggagtagtacccatg

SEQ ID NO: 39 - DNA, 24 bp, synthetic construct
  source 1..24: mol_type = other DNA
cataggcccctctccttcagcttg

SEQ ID NO: 40 - DNA, 59 bp, synthetic construct
  source 1..59: mol_type = other DNA
cgcatgggtactactcctgtctggtcagcttattgctggagaatcttgggataacgctg

SEQ ID NO: 41 - DNA, 47 bp, synthetic construct
  source 1..47: mol_type = other DNA
ccaagctgaaggagaggggcctatgaccatgaacgatctccctaacc

SEQ ID NO: 42 - DNA, 24 bp, synthetic construct
  source 1..24: mol_type = other DNA
cacaggcccctctccttcagcttg

SEQ ID NO: 43 - DNA, 47 bp, synthetic construct
  source 1..47: mol_type = other DNA
ggcaaatggaagggaattgagaatgaccatgaacgatctccctaacc

SEQ ID NO: 44 - DNA, 47 bp, synthetic construct
  source 1..47: mol_type = other DNA
caagctgaaggagaggggcctgtgaagcgcatggtcgtggggattac

SEQ ID NO: 45 - DNA, 38 bp, synthetic construct
  source 1..38: mol_type = other DNA
cattctcaattcccttccatttgcccagcacccggaac

SEQ ID NO: 46 - DNA, 54 bp, synthetic construct
  source 1..54: mol_type = other DNA
gtttctgcggactggctttctacgtgttccgttcattacttcacccagcagggc

SEQ ID NO: 47 - DNA, 51 bp, synthetic construct
  source 1..51: mol_type = other DNA
ggtctcaaaggctgaacggaaagggcaaggcgatcttctactacgaaaagg

SEQ ID NO: 48 - DNA, 51 bp, synthetic construct
  source 1..51: mol_type = other DNA
ccttttcgtagtagaagatcgccttgccctttccgttcagcctttgagacc

SEQ ID NO: 49 - DNA, 54 bp, synthetic construct
  source 1..54: mol_type = other DNA
cgattcattaatgcagctggcacgacagcgaaggtctcatctgatcgaagagcg

SEQ ID NO: 50 - DNA, 49 bp, synthetic construct
  source 1..49: mol_type = other DNA
gtttctgcggactggctttctacgtgttctcgccgcaagctaggaccgc

SEQ ID NO: 51 - DNA, 48 bp, synthetic construct
  source 1..48: mol_type = other DNA
tcgttgcttgccacatcgaagatcgacggccacaggactaagcgttgc

SEQ ID NO: 52 - DNA, 48 bp, synthetic construct
  source 1..48: mol_type = other DNA
gcaacgcttagtcctgtggccgtcgatcttcgatgtggcaagcaacga

SEQ ID NO: 53 - DNA, 56 bp, synthetic construct
  source 1..56: mol_type = other DNA
cgattcattaatgcagctggcacgacagcgttatgtggtgatttcagcgatcgtcg

SEQ ID NO: 54 - DNA, 51 bp, synthetic construct
  source 1..51: mol_type = other DNA
cggactactcccgttatgtggtgatcagcctttgagaccattcctaaagaa

SEQ ID NO: 55 - DNA, 50 bp, synthetic construct
  source 1..50: mol_type = other DNA
caaggcgatcttctactacgaaaaggcgctgaacgaccgcttcatgacgg

SEQ ID NO: 56 - DNA, 49 bp, synthetic construct
  source 1..49: mol_type = other DNA
tttcgtagtagaagatcgccttgctcaggcctgggcgcgggtgcgttcc

SEQ ID NO: 57 - DNA, 51 bp, synthetic construct
  source 1..51: mol_type = other DNA
tcaccacataacgggagtagtccgatgcctgccaccttcgccagttccgat

SEQ ID NO: 58 - DNA, 51 bp, synthetic construct
  source 1..51: mol_type = other DNA
tttcgtagtagaagatcgccttgtcattccttgacgcgggcgcgcttgccg

SEQ ID NO: 59 - DNA, 51 bp, synthetic construct
  source 1..51: mol_type = other DNA
tcaccacataacgggagtagtccgatgtcgaagaacccgtcgcagcagtcg

SEQ ID NO: 60 - DNA, 49 bp, synthetic construct
  source 1..49: mol_type = other DNA
gtattttcagagcgcgatcgcaggaatgaggctcatcgtgggcatgacg

SEQ ID NO: 61 - DNA, 51 bp, synthetic construct
  source 1..51: mol_type = other DNA
ctaactttgttattttcggctttctgtcagatgccgtcgttgttctcctgg

SEQ ID NO: 62 - DNA, 49 bp, synthetic construct
  source 1..49: mol_type = other DNA
gtattttcagagcgcgatcgcaggaatgcagaaccccatcaacgacctc

SEQ ID NO: 63 - DNA, 50 bp, synthetic construct
  source 1..50: mol_type = other DNA
ctaactttgttattttcggctttctggctcacaggagcggaggaatcgtg

SEQ ID NO: 64 - DNA, 42 bp, synthetic construct
  source 1..42: mol_type = other DNA
gtattttcagagcgcgatcgcaatgcctgccaccttcgccag

SEQ ID NO: 65 - DNA, 45 bp, synthetic construct
  source 1..45: mol_type = other DNA
ctaactttgttattttcggctttctgtcaggcctgggcgcgggtg

SEQ ID NO: 66 - DNA, 47 bp, synthetic construct
  source 1..47: mol_type = other DNA
gtattttcagagcgcgatcgcaatgaccatgaacgatctccctaacc

SEQ ID NO: 67 - DNA, 52 bp, synthetic construct
  source 1..52: mol_type = other DNA
ctaactttgttattttcggctttctgttattgctggagaatcttgggataac

SEQ ID NO: 68 - DNA, 48 bp, synthetic construct
  source 1..48: mol_type = other DNA
gtattttcagagcgcgatcgcaggaatgtcgaagaacccgtcgcagca

SEQ ID NO: 69 - DNA, 48 bp, synthetic construct
  source 1..48: mol_type = other DNA
ctaactttgttattttcggctttctgttccttgacgcgggcgcgcttg